(19) United States
(12) Patent Application Publication
(10) Pub. No.: US 2006/0004004 A1
(43) Pub. Date: Jan. 5, 2006

(54) Novel gamma secretase inhibitors

(75) Inventors: Theodros Asberom, West Orange, NJ (US); John W. Clader, Cranford, NJ (US); Hubert B. Josien, Hoboken, NJ (US); Mark D. McBriar, Clinton, NJ (US); Dmitri A. Pissarnitski, Scotch Plains, NJ (US); Zhiqiang Zhao, Scotch Plains, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/098,745

(22) Filed: Apr. 4, 2005

Related U.S. Application Data
(60) Provisional application No. 60/559,529, filed on Apr. 5, 2004.

(51) Int. Cl. A61K 31/5377; A61K 31/496; A61K 31/4545; C07D 413/14; C07D 41/14

(52) U.S. Cl. 514/235.2; 514/316; 514/326; 514/253.11; 544/124; 544/360; 546/187

(57) ABSTRACT

Gamma-secretase inhibitors of the formula: 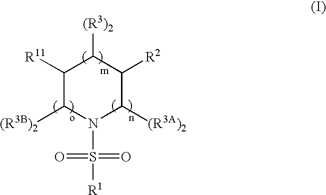 are useful in treating various neurodegenerative diseases, wherein, for example:
R¹ includes unsubstituted or substituted aryl or heteroaryl groups;
R² includes —C(O)—Y, -alkylene-C(O)—Y, -alkylene-cycloalkylene-C(O)—Y, -cycloalkylene-alkylene-C(O)—Y, -alkylene-cycloalkylene-alkylene-C(O)—Y, -cycloalkylene-C(O)—Y, —S(O)—Y, -alkylene-S(O)—Y, -alkylene-cycloalkylene-S(O)—Y, -cycloalkylene-alkylene-S(O)—Y, -alkylene-cycloalkylene-alkylene-S(O)—Y, -cycloalkylene-S(O)—Y, —S(O₂)—Y, -alkylene-S(O₂)—Y, -alkylene-cycloalkylene-S(O₂)—Y, -cycloalkylene-alkylene-S(O₂)—Y, -alkylene-cycloalkylene-alkylene-S(O₂)—Y, and -cycloalkylene-S(O₂)—Y, wherein Y is as defined herein, and each of said alkylene or cycloalkylene may be unsubstituted or substituted as provided herein;
each R³ is independently includes H, alkyl, —O-alkyl, —OH, —N(R⁹)₂, acyl, and aroyl; or
the moiety (R³)₂, together with the ring carbon atom to which it is shown attached in formula I, defines a carbonyl group, —C(O)—;
each R^{3A} and R^{3B} independently includes H, or alkyl;
R¹¹ includes aryl, heteroaryl, alkyl, cycloalkyl, arylalkyl, arylcycloalkyl, heteroarylalkyl, heteroarylcycloalkyl, arylheterocycloalkyl, or alkoxyalkyl. One or more of the compounds of formula I, or pharmaceutically acceptable salts, solvates, and/or esters, or compositions comprised thereof, may be used to treat, e.g., Alzheimer's Disease.

This application claims the benefit of U.S. Provisional Application No. 60/559,529, filed Apr. 5, 2004.

### BACKGROUND OF THE INVENTION

WO 00/50391, published Aug. 13, 2000, discloses compounds having a sulfonamide moiety that are useful for the treatment and prevention of Alzheimer's Disease and other diseases relating to the deposition of amyloid protein.

In view of the present interest in the treatment or prevention of neurodegenerative diseases, such as Alzheimer's Disease, a welcome contribution to the art would be compounds for use in such treatment or prevention. This invention provides such a contribution.

### SUMMARY OF THE INVENTION

This invention provides compounds that are inhibitors (e.g., antagonists) of gamma-secretase and have the formula I: 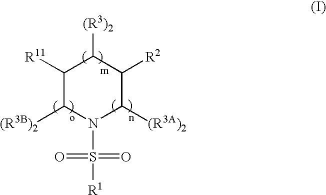
or a pharmaceutically acceptable salt, solvate and/or ester thereof, wherein:

R¹ is selected from the group consisting of unsubstituted aryl, aryl substituted with one or more R⁵ groups, unsubstituted heteroaryl, and heteroaryl substituted with one or more R⁵ groups;

R² is selected from the group consisting of —C(O)—Y, -alkylene-C(O)—Y, -alkylene-cycloalkylene-C(O)—Y, -cycloalkylene-alkylene-C(O)—Y, -alkylene-cycloalkylene-alkylene-C(O)—Y, -cycloalkylene-C(O)—Y, —S(O)—Y, -alkylene-S(O)—Y, -alkylene-cycloalkylene-S(O)—Y, -cycloalkylene-alkylene-S(O)—Y, -alkylene-cycloalkylene-alkylene-S(O)—Y, -cycloalkylene-S(O)—Y, -S(O₂)—Y, -alkylene-S(O₂)—Y, -alkylene-cycloalkylene-S(O₂)—Y, -cycloalkylene-alkylene-S(O₂)—Y, -alkylene-cycloalkylene-alkylene-S(O₂)—Y, and -cycloalkylene-S(O₂)—Y; wherein each of said alkylene or cycloalkylene are unsubstituted or optionally substituted with one or more hydroxy groups, with the proviso that no hydroxy group is bonded to a carbon atom which is also bonded to a sulfur atom;

each R³ is independently selected from the group consisting of H, alkyl, —O-alkyl, —OH, —N(R⁹)₂, acyl, and aroyl; or

the moiety (R³)₂, together with the ring carbon atom to which it is shown attached in formula I, defines a carbonyl group, —C(O)—, with the proviso that when m is an integer greater than 1, at most one carbonyl group is present in the ring shown in formula I;

each R^{3A} and R^{3B} is independently selected from the group consisting of H and alkyl;

R⁵ is independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, and —N(R⁶)C(O)NR⁶R⁷;

Y is selected from the group consisting of —NR⁶R⁷, —N(R¹²)(CH₂)_{b}NR⁶R⁷ (wherein b is an integer of from 2-6), aryl, heteroaryl, alkyl, cycloalkyl, heterocycloalkyl, arylalkyl, arylcycloalkyl, heteroarylalkyl, heteroarylcycloalkyl, arylheterocycloalkyl, aryl alkyl heterocycloalkyl, substituted aryl, substituted heteroaryl, substituted arylalkyl, substituted arylcycloalkyl, substituted heteroarylalkyl, substituted heteroarylcycloalkyl, substituted arylheterocycloalkyl, and substituted heterocycloalkyl alkyl; wherein the aryl or heteroaryl moiety in said substituted aryl, substituted heteroaryl, substituted arylalkyl, substituted arylcycloalkyl, substituted heteroarylalkyl, substituted heteroarylcycloalkyl, substituted arylheterocycloalkyl, or substituted heterocycloalkyl alkyl groups of said Y group are substituted with one or more substituents independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, —N(R⁶)C(O)NR⁶R⁷, and alkyl; or Y is selected from the group consisting of:

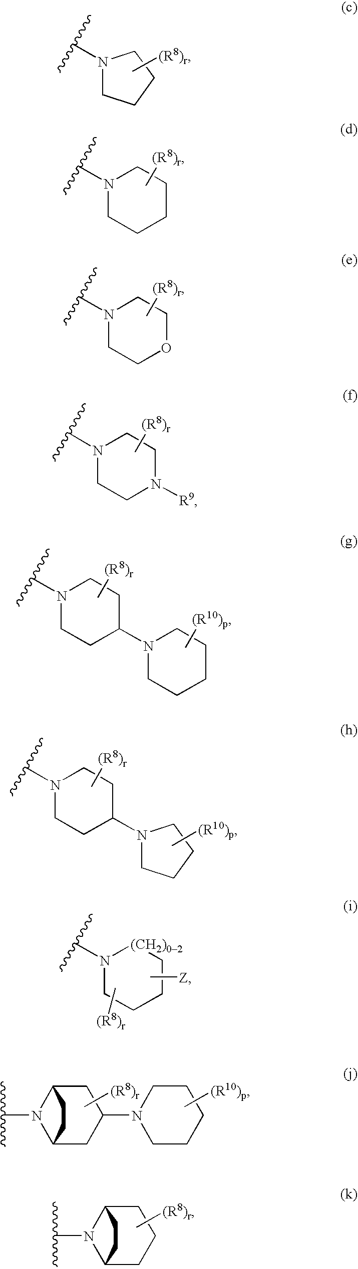

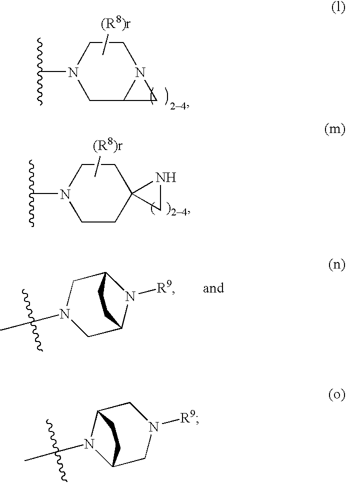

R⁶ and R⁷ are independently selected from the group consisting of H, alkyl, alkyl substituted with 1 to 4 hydroxy groups, cycloalkyl, arylalkyl, heteroarylalkyl, 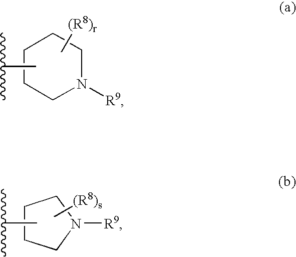
and heterocycloalkyl, with the proviso that if R⁶ and/or R⁷ are alkyl substituted with 1 to 4 hydroxy groups, none of the hydroxy groups are bonded to a carbon to which a nitrogen is also bonded;

R⁸ is independently selected from the group consisting of H, —OH, alkyl, —O-alkyl, alkyl substituted with 1 to 4 hydroxy groups, and —C(O)O-alkyl; or if r is greater than 1 and at least two R⁸ groups are selected from the group consisting of alkyl, —O-alkyl, alkyl substituted with 1 to 4 hydroxy groups, and —C(O)O-alkyl, then the two R⁸ groups, together with the ring carbon atom or atoms to which they are attached, define a ring;

each R⁹ is independently selected from the group consisting of H, alkyl, alkyl substituted with 1 to 4 hydroxy groups, cycloalkyl, cycloalkyl substituted with 1 to 4 hydroxy groups, arylalkyl, heteroarylalkyl, —C(O)O-alkyl, -alkylene-O-alkylene-OH, aryl substituted with one or more R⁵ groups, heteroaryl substituted with one or more R⁵ groups, unsubstituted heteroaryl, unsubstituted aryl, -alkylene-C(O)O-alkyl, —(SO₂)-alkyl, —(SO₂)-aryl, and hydroxyalkyl-O-alkyl, with the proviso that when R⁹ is alkyl substituted with 1 to 4 hydroxy groups, none of the hydroxy groups are bonded to a carbon to which a nitrogen is also bonded;

each R¹⁰ is independently selected from the group consisting of H and alkyl;

R¹¹ is selected from the group consisting of aryl, substituted aryl, heteroaryl, alkyl, cycloalkyl, arylalkyl, arylcycloalkyl, heteroarylalkyl, heteroarylcycloalkyl, arylheterocycloalkyl, alkoxyalkyl, substituted heteroaryl, substituted arylalkyl, substituted arylcycloalkyl, substituted heteroarylalkyl, and substituted arylheterocycloalkyl; wherein the aryl or heteroaryl moiety in said substituted heteroaryl, substituted arylalkyl, substituted arylcycloalkyl, substituted heteroarylalkyl, and substituted arylheterocycloalkyl of said R¹¹ group is substituted with one or more substituents independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, and —N(R⁶)C(O)NR 6R⁷;

R¹² is selected from the group consisting of H, alkyl, aryl, and aryl substituted with one or more substituents independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, and —N(R⁶)C(O)NR⁶R⁷;

m is an integer of from 0 to 3, and if m is greater than 1, the m moieties can be the same or different from one another;

n is an integer of from 0 to 3, and if n is greater than 1, the n moieties can be the same or different from one another;

o is an integer of from 0 to 3, and if o is greater than 1, the o moieties can be the same or different from one another;

with the proviso that m+n+o is 1, 2, 3 or 4;

p is an integer of from 0 to 4, and if p is greater than 1, the p moieties can be the same or different from one another;

r is an integer of from 0 to 4, and if r is greater than 1, the r moieties can be the same or different from one another;

s is an integer of from 0 to 3, and if s is greater than 1, the s moieties can be the same or different from one another; and

Z is selected from the group consisting of heterocycloalkyl, substituted heterocycloalkyl, —NH₂, —NH(alkyl), —N(alkyl)₂ wherein each alkyl is the same or different, —NH(cycloalkyl), —NH(substituted cycloalkyl), —N(alkyl)(cycloalkyl), —N(alkyl)(substituted cycloalkyl), —NH(aralkyl), —NH(substituted aralkyl), —N(alkyl)(aralkyl), —NH(heterocycloalkyl), —NH(substituted heterocycloalkyl), —N(alkyl)(heterocycloalkyl), —N(alkyl)(substituted heterocycloalkyl), —NH(heteroaralkyl), —NH(substituted heteroaralkyl), —NH-alkylene-(cycloalkyl), —NH-alkylene-(substituted cycloalkyl), —N(alkyl)-alkylene-(cycloalkyl), —N(alkyl)-alkylene-(substituted cycloalkyl), —NH-alkylene-(heterocycloalkyl), —NH-alkylene-(substituted heterocycloalkyl), —N(alkyl)-alkylene-(heterocycloalkyl), —N(alkyl)-alkylene-(substituted heterocycloalkyl), benzo-fused heterocycloalkyl, substituted benzo-fused heterocycloalkyl, H, and —N(hydroxyalkyl)₂, wherein each alkyl may be the same or different; wherein said substituted cycloalkyl, substituted heterocycloalkyl, substituted aryl, or substituted heteroaryl moiety of group Z is substituted with one or more substituents independently selected from the group consisting of alkyl, —OH, alkoxy, —OC(O)-alkyl, —OC(O)-aryl, —NH₂, —NH(alkyl), —N(alkyl)₂ wherein each alkyl is the same or different, —NHC(O)-alkyl, —N(alkyl)C(O)-alkyl, —NHC(O)-aryl, —N(alkyl)C(O)-aryl, —C(O)-alkyl, —C(O)-aryl, —C(O)NH₂, —C(O)NH(alkyl), —C(O)N(alkyl)₂ wherein each alkyl is the same or different, —C(O)O-alkyl, -alkylene-C(O)O-alkyl, piperidinyl, pyrrolidinyl, aryl, heteroaryl, and —O—CH₂CH₂—O— wherein both oxygen atoms are bound to the same carbon atom, and provided that the aryl and heteroaryl moieties of said Z group are not substituted with said —O—CH₂CH₂—O— group.

This invention also provides a pharmaceutical composition comprising an effective amount of one or more compounds of formula I and at least one pharmaceutically acceptable carrier.

This invention also provides a method for inhibiting gamma-secretase comprising administering an effective (i.e., therapeutically effective) amount of one or more compounds of formula I to a patient in need of treatment.

This invention also provides a method of treating one or more neurodegenerative diseases comprising administering an effective (i.e., therapeutically effective) amount of one or more compounds of formula I to a patient in need of treatment.

This invention also provides a method of inhibiting the deposition of amyloid protein (e.g., amyloid beta protein) in, on or around neurological tissue (e.g., the brain) comprising administering an effective (i.e., therapeutically effective) amount of one or more compounds of formula I to a patient in need of treatment.

This invention also provides a method of treating Alzheimer's disease comprising administering an effective (i.e., therapeutically effective) amount of one or more compounds of formula I to a patient in need of treatment.

### DETAILED DESCRIPTION OF THE INVENTION

In one embodiment, the present invention provides for compounds of formula I, as described above.

In another embodiment of the compounds of formula I, R² is —(C₀-C₁₂)alkylene-C(O)—Y, —(C₀-C₆)alkylene-(C₃-C₆)cycloalkylene-(C₀-C₆)alkylene-C(O)—Y, —(C₀-C₁₂)alkylene-S(O)—Y, —(C₀-C₆)alkylene-(C₃-C₆)cycloalkylene-(C₀-C₆)alkylene-S(O)—Y, —(C₀-C₁₂)alkylene-S(O₂)—Y, or —(C₀-C₆)alkylene-(C₃-C₆)cycloalkylene-(C₀-C₆)alkylene-S(O)₂—Y.

In another embodiment of the compounds of formula I, R² is —(C₃-C₆)cycloalkylene-C(O)—Y.

In another embodiment of the compounds of formula I, R² is -cyclopropylene-C(O)—Y.

In another embodiment of the compounds of formula I, R² is —(C₃-C₆)cycloalkylene-(C₀-C₆)alkylene-C(O)—Y.

In another embodiment of the compounds of formula I, R² is —(C₃-C₆)cycloalkylene-(C₀-C₆)alkylene(OH)—C(O)—Y.

In another embodiment of the compounds of formula I, R² is -cyclopropylene-CH₂—C(O)—Y.

In another embodiment of the compounds of formula I, R² is -cyclopropylene-CH(OH)—C(O)—Y.

In another embodiment of the compounds of formula I, R² is —(C₃-C₆)cycloalkylene-S(O₂)—Y.

In another embodiment of the compounds of formula I, R² is -cyclopropylene-S(O₂)—Y.

In another embodiment of the compounds of formula I, R² is —(C₃-C₆)cycloalkylene-(C₀-C₆)alkylene-S(O₂)—Y.

In another embodiment of the compounds of formula I, R² is -cyclopropylene-CH₂—S(O₂)—Y.

In another embodiment of the compounds of formula I, Y is:

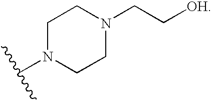

In another embodiment of the compounds of formula I, Y is:

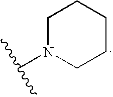

In another embodiment of the compounds of formula I, Y is:

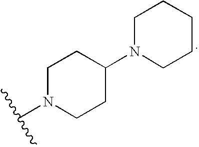

In another embodiment of the compounds of formula I, Y is:

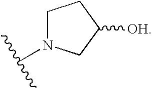

In another embodiment of the compounds of formula I, Y is:

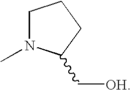

In another embodiment of the compounds of formula I, Y is:

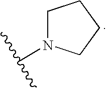

In another embodiment of the compounds of formula I, Y is:

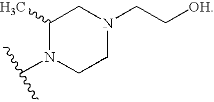

In another embodiment of the compounds of formula I, Y is:

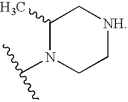

In another embodiment of the compounds of formula I, Y is:

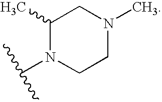

In another embodiment of the compounds of formula I, Y is:

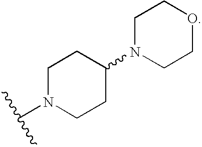

In another embodiment of the compounds of formula I, Y is:

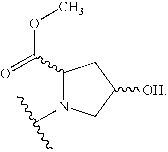

In another embodiment of the compounds of formula I, Y is:

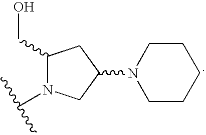

In another embodiment of the compounds of formula I, Y is:

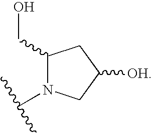

In another embodiment of the compounds of formula I, Y is:

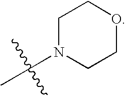

In another embodiment of the compounds of formula I, Y is:

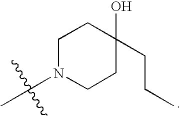

In another embodiment of the compounds of formula I, Y is:

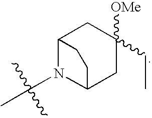

In another embodiment of the compounds of formula I, Y is:

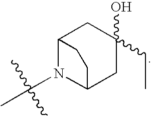

In another embodiment of the compounds of formula I, Y is:

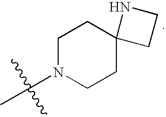

In another embodiment of the compounds of formula I, Y is:

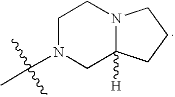

In another embodiment of the compounds of formula I, Y is:

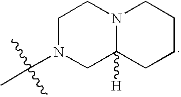

In another embodiment of the compounds of formula I, Y is:

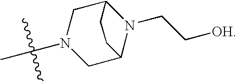

In another embodiment of the compounds of formula I, Y is:

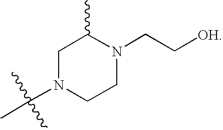

In another embodiment of the compounds of formula I, Y is

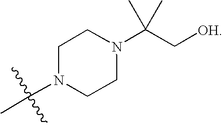

In another embodiment of the compounds of formula I, Y is —N(CH₂CH₂OH)₂.

In another embodiment of the compounds of formula I, R² is —(C₀-C₁₂)alkylene-C(O)—Y, —(C₀-C₆)alkylene-(C₃-C₆)cycloalkylene-(C₀-C₆)alkylene-C(O)—Y, —(C₀-C₁₂)alkylene-S(O)—Y, —(C₀-C₆)alkylene-(C₃-C₆)cycloalkylene-(C₀-C₆)alkylene-S(O)—Y, —(C₀-C₁₂)alkylene-S(O₂)—Y, or —(C₀-C₆)alkylene-(C₃-C₆)cycloalkylene-(C₀-C₆)alkylene-S(O₂)—Y and Y is selected from the group consisting of:

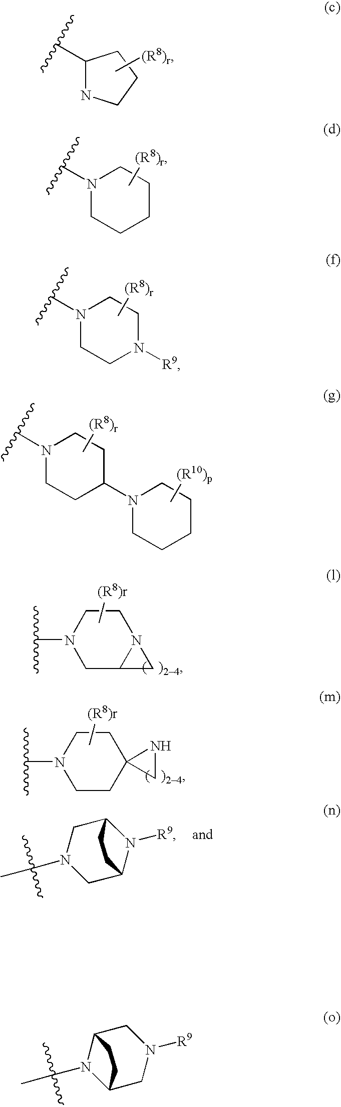

In another embodiment of the compounds of formula I, R² is:

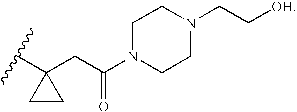

In another embodiment of the compounds of formula I, R² is:

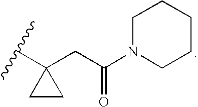

In another embodiment of the compounds of formula I, R² is:

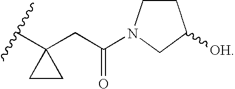

In another embodiment of the compounds of formula I, R² is:

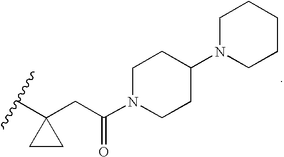

In another embodiment of the compounds of formula I, R² is:

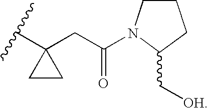

In another embodiment of the compounds of formula I, R² is:

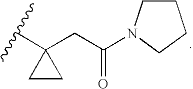

In another embodiment of the compounds of formula I, R² is:

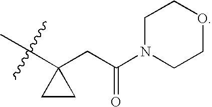

In another embodiment of the compounds of formula I, R² is:

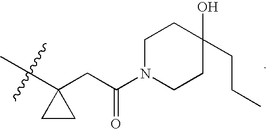

In another embodiment of the compounds of formula I, R² is:

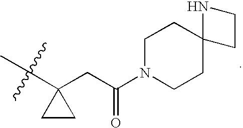

In another embodiment of the compounds of formula I, R² is:

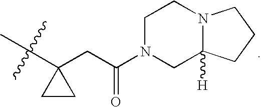

In another embodiment of the compounds of formula I, R² is:

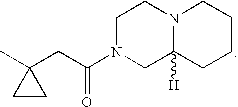

In another embodiment of the compounds of formula I, R² is:

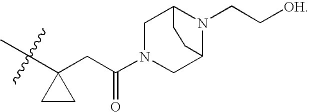

In another embodiment of the compounds of formula I, R² is:

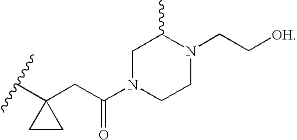

In another embodiment of the compounds of formula I, R² is:

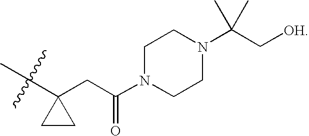

In another embodiment of the compounds of formula I, R² is:

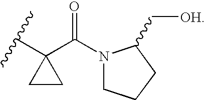

In another embodiment of the compounds of formula I, R² is:

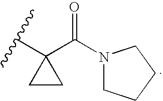

In another embodiment of the compounds of formula I, R² is:

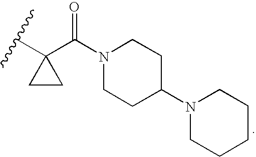

In another embodiment of the compounds of formula I, R² is:

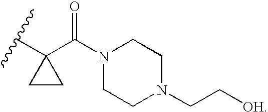

In another embodiment of the compounds of formula I, R² is:

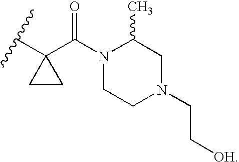

In another embodiment of the compounds of formula I, R² is:

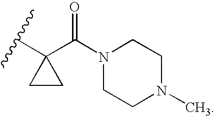

In another embodiment of the compounds of formula I, R² is:

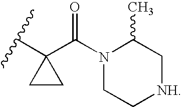

In another embodiment of the compounds of formula I, R² is:

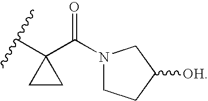

In another embodiment of the compounds of formula I, R² is:

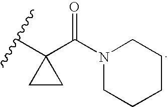

In another embodiment of the compounds of formula I, R² is:

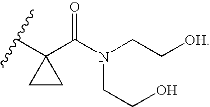

In another embodiment of the compounds of formula I, R² is:

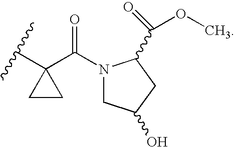

In another embodiment of the compounds of formula I, R² is:

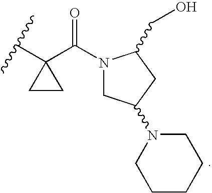

In another embodiment of the compounds of formula I, R² is:

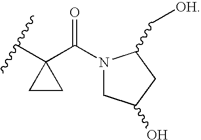

In another embodiment of the compounds of formula I, R² is:

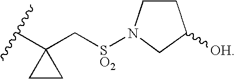

In another embodiment of the compounds of formula I, R² is:

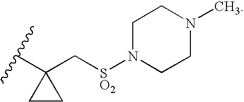

In another embodiment of the compounds of formula I, R² is:

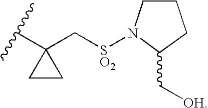

In another embodiment of the compounds of formula I, R² is:

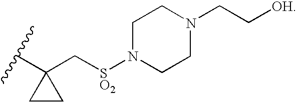

In another embodiment of the compounds of formula I, R² is:

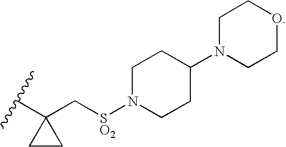

In another embodiment of the compounds of formula I, R² is:

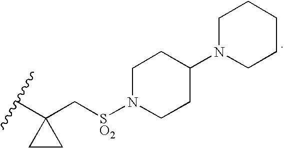

In another embodiment of the compounds of formula I, R² is:

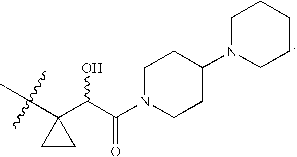

In another embodiment of the compounds of formula I, R² is:

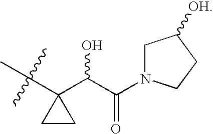

In another embodiment of the compounds of formula I, R² is:

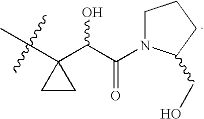

In another embodiment of the compounds of formula I, R² is:

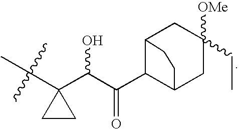

In another embodiment of the compounds of formula I, R² is:

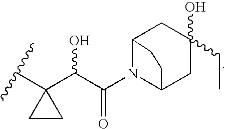

In another embodiment of the compounds of formula I, R² is —(C₀-C₁₂)alkylene-C(O)—Y, —(C₀-C₆)alkylene-(C₃-C₆)cycloalkylene-(C₀-C₆)alkylene-C(O)—Y, —(C₀-C₁₂)alkylene-S(O)—Y, —(C₀-C₆)alkylene-(C₃-C₆)cycloalkylene-(C₀-C₆)alkylene-S(O)—Y, —(C₀-C₁₂)alkylene-S(O₂)—Y, or —(C₀-C₆)alkylene-(C₃-C₆)cycloalkylene-(C₀-C₆)alkylene-S(O₂)—Y;

Y is:

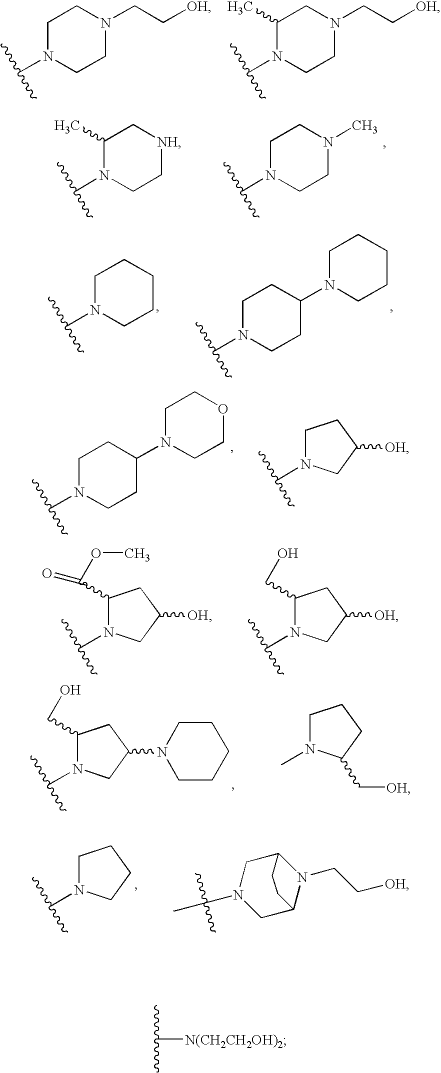

each R³ of (R³)₂ is independently selected from the group consisting of H, —OH, —(C₁-C₆)alkyl, —O—(C₁-C₆)alkyl, —N(R⁹)₂, —C₁-C₆)acyl, and —(C₇-C₁₃)aroyl;

each R^{3A} and R^{3B} is independently selected from the group consisting of H and —C₁-C₆)alkyl;

R⁵ is independently selected from the group consisting of halo, —OH, —CF₃, and —O—(C₁-C₆)alkyl;

R¹¹ is selected from the group consisting of —(C₆-C₁₂)aryl, substituted —(C₆-C₁₂)aryl, —(C₆-C₁₂)heteroaryl, and substituted —(C₆-C₁₂)heteroaryl, wherein said substituted —(C₆-C₁₂)aryl and substituted —(C₆-C₁₂)heteroaryl are substituted with one or more halo, —CF₃, —OH, or —O—(C₁-C₆)alkyl groups;

m is 0 or 1;

n is 0 or 1; and

o is 0 or 1.

In yet another embodiment of the compounds of formula I, R² is selected from the group consisting of —(C₀-C₁₂)alkylene-C(O)—Y and —(C₀-C₆)alkylene-(C₃-C₆)cycloalkylene-(C₀-C₆)alkylene-C(O)—Y;

Y is selected from the group consisting of:

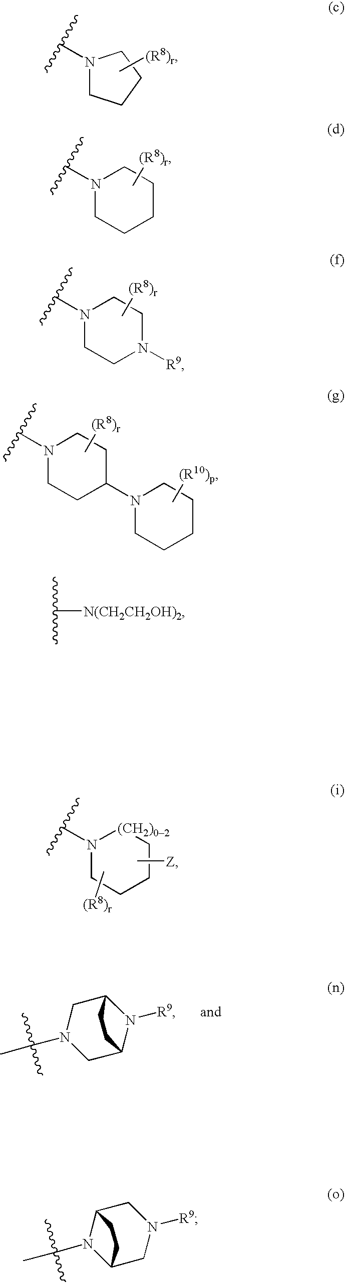

each R³ of (R³)₂ is independently selected from the group consisting of H, —OH, —(C₁-C₆)alkyl, —O—(C₁-C₆)alkyl, —N(R⁹)₂, —(C₁-C₆)acyl, and —(C₇-C₁₃)aroyl; or

(R³)₂ together with the ring carbon to which it is shown attached in formula I defines a carbonyl group, with the proviso that when m is an integer greater than 1, at most one carbonyl group is present in the ring shown in formula I;

each R^{3A} and R^{3B} is independently selected from the group consisting of H and (C₁-C₆)alkyl;

R⁵ is independently selected from the group consisting of halo, —OH, —CF₃, and —O—(C₁-C₆)alkyl;

R⁸ is independently selected from the group consisting of H, —OH, —(C₁-C₆)alkyl, —O—(C₁-C₆)alkyl, —(C₁-C₆)alkyl substituted with a hydroxy group, and —C(O)O—(C₁-C₆)alkyl, with the proviso that if R⁸ is —OH or —(C₁-C₆)alkyl substituted with a hydroxy group;

R⁹ is independently selected from the group consisting of H, alkyl, and —(C₁-C₆)alkyl substituted with a hydroxy group, with the proviso that if R⁹ is —(C₁-C₆)alkyl substituted with a hydroxy group, no hydroxy group is bonded to a carbon atom which is also bonded to a nitrogen atom;

R¹¹ is selected from the group consisting of (C₆-C₁₂)aryl, substituted (C₆-C₁₂)aryl, (C₆-C₁₂)heteroaryl, and substituted (C₆-C₁₂)heteroaryl, wherein said substituted (C₆-C₁₂)aryl and substituted (C₆-C₁₂)heteroaryl are substituted with one or more halo, —CF₃, —OH, or —O—(C₁-C₆)alkyl groups;

Z is selected from the group consisting of heterocycloalkyl:

m is 0 or 1;

n is 0 or 1; and

o is 0 or 1.

In yet another embodiment of the compounds of formula I, R¹ is unsubstituted aryl or aryl substituted with one or more R⁵ groups.

In yet another embodiment of the compounds of formula I, R¹ is phenyl.

In yet another embodiment of the compounds of formula I, R¹ is phenyl substituted with one or more R⁵ groups.

In yet another embodiment of the compounds of formula I, R¹ is phenyl substituted with one or more halo atoms.

In yet another embodiment of the compounds of formula I, R¹ is phenyl substituted with one halo atom.

In yet another embodiment of the compounds of formula I, R¹ is phenyl substituted with chloro (e.g., p-chlorophenyl).

In yet another embodiment of the compounds of formula I, R¹ is unsubstituted heteroaryl (e.g., pyridyl, pyrimidyl, pyridazyl, pyrazyl) or heteroaryl substituted with one or more R⁵ groups.

In yet another embodiment of the compounds of formula I, R² is —C(O)Y, —(C₁-C₆)alkylene-C(O)—Y, —(C₃-C₆)cycloalkylene-C(O)—Y, —(C₃-C₆)cycloalkylene-(C₁-C₆)alkylene-C(O)—Y, or —(C₁-C₆)alkylene-(C₃-C₆)cycloalkylene-(C₁-C₆)alkylene-C(O)—Y.

In yet another embodiment of the compounds of formula I, R² is —(C₃-C₆)cycloalkylene-C(O)—Y or —(C₃-C₆)cycloalkylene-(C₁-C₆)alkylene-C(O)—Y.

In yet another embodiment of the compounds of formula I, R² is cyclopropylene-(C₁-C₆)alkylene-C(O)—Y or cyclopropylene-C(O)—Y.

In yet another embodiment of the compounds of formula I, R² is cyclopropylene-CH₂—C(O)—Y or cyclopropylene-C(O)—Y.

In yet another embodiment of the compounds of formula I, R² is —S(O)Y, —(C₁-C₆)alkylene-S(O)—Y, —(C₃-C₆)cycloalkylene-S(O)—Y, —(C₃-C₆)cycloalkylene-(C₁-C₆)alkylene-S(O)—Y, or —(C₁-C₆)alkylene-(C₃-C₆)cycloalkylene-(C₁-C₆)alkylene-S(O)—Y.

In yet another embodiment of the compounds of formula I, R² is —(C₃-C₆)cycloalkylene-S(O)—Y or —(C₃-C₆)cycloalkylene-(C₁-C₆)alkylene-S(O)—Y.

In yet another embodiment of the compounds of formula I, R² is -cyclopropylene-(C₁-C₆)alkylene-S(O)—Y or -cyclopropylene-S(O)—Y.

In yet another embodiment of the compounds of formula I, R² is -cyclopropylene-CH₂—S(O)—Y or -cyclopropylene-S(O)—Y.

In yet another embodiment of the compounds of formula I, R² is —S(O₂)Y, —(C₁-C₆)alkylene-S(O₂)—Y, —(C₃-C₆)cycloalkylene-S(O₂)—Y, —(C₃-C₆)cycloalkylene-(C₁-C₆)alkylene-S(O₂)—Y, or —(C₁-C₆)alkylene-(C₃-C₆)cycloalkylene-(C₁-C₆)alkylene-S(O₂)—Y.

In yet another embodiment of the compounds of formula I, R² is —(C₃-C₆)cycloalkylene-S(O₂)—Y or —(C₃-C₆)cycloalkylene-(C₁-C₆)alkylene-S(O₂)—Y.

In yet another embodiment of the compounds of formula I, R² is -cyclopropylene-(C₁-C₆)alkylene-S(O₂)—Y or -cyclopropylene-S(O₂)—Y.

In yet another embodiment of the compounds of formula I, R² is cyclopropylene-CH₂-S(O₂)—Y or -cyclopropylene-S(O₂)—Y.

In yet another embodiment of the compounds of formula I, each R³ of (R³)₂ is independently H, —OH, —NH₂, —NH(SO₂)-alkyl, —NH(SO₂)-aryl, —(C₂-C₆)acyl (e.g., acetyl), or (C₇-C₁₃)aroyl (e.g., benzoyl).

In yet another embodiment of the compounds of formula I, each R³ of (R³)₂ is H.

In yet another embodiment of the compounds of formula I, (R³)₂ together with the ring carbon to which it is shown attached in formula I defines a carbonyl group, with the proviso that when m is an integer greater than 1, at most one carbonyl group is present in the ring shown in formula I.

In yet another embodiment of the compounds of formula I, (R³)₂ together with the ring carbon to which it is shown attached in formula I defines a carbonyl group, and m is 1.

In yet another embodiment of the compounds of formula I, each R^{3A} and R^{3B} is independently H or (C₁-C₆)alkyl (e.g., methyl, ethyl, n-propyl, i-propyl, n-butyl, sec-butyl, t-butyl, n-pentyl, neo-pentyl or hexyl).

In yet another embodiment of the compounds of formula I, each R³A and R^{3B} is H.

In yet another embodiment of the compounds of formula I, each R⁵is independently halo (e.g., Cl), —CF₃, —OH, alkoxy (e.g., methoxy), —OCF₃, —CN, —NH₂, —C(O)O-alkyl (e.g., —C(O)O—CH₃ or—C(O)O—CH₂CH₃), —OC(O)-alkyl (e.g., —OC(O)—CH₃), —C(O)O-aryl (e.g., —C(O)O-phenyl), —OC(O)-aryl (e.g., —OC(O)-phenyl), —C(O)NR⁶R⁷ (e.g., —C(O)N(CH₃)₂), -alkylene-NR⁶R⁷(e.g., —CH₂—N(CH₃)₂ or —CH₂CH₂—N(CH₃)₂), —N(R⁶)C(O)-alkyl (e.g., —N(CH₃)C(O)—CH₃ or —NHC(O)—CH₃), —N(R⁶)C(O)-aryl (e.g., —N(CH₃)C(O)-phenyl or —NHC(O)-phenyl), —N(R⁶)C(O)-heteroaryl (e.g., —N(CH₃)C(O)-pyridyl or —NHC(O)-pyridyl), or —N(R⁶)C(O)NR⁶R⁷ (e.g., —N(CH₃)C(O)N(CH₃)₂ or —NHC(O)N(CH₃)₂).

In yet another embodiment of the compounds of formula I, Y is selected from the group consisting of:

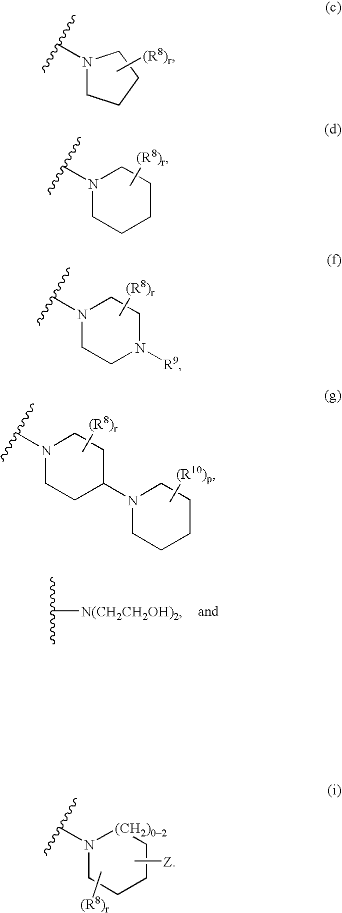

In yet another embodiment of the compounds of formula I, Y is:

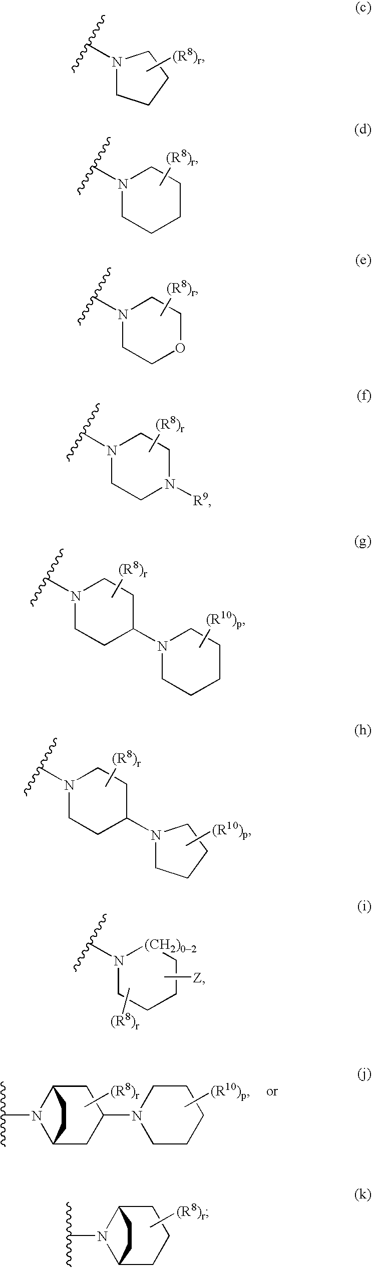

r is 2;

one R⁸ is —(C₁-C₆)alkyl, and the second R⁸ is —O—(C₁-C₆)alkyl, and the two R⁸ groups, together with the ring carbon atoms to which they are attached, form a polycyclic ring structure.

In yet another embodiment of the compounds of formula I, Y is:

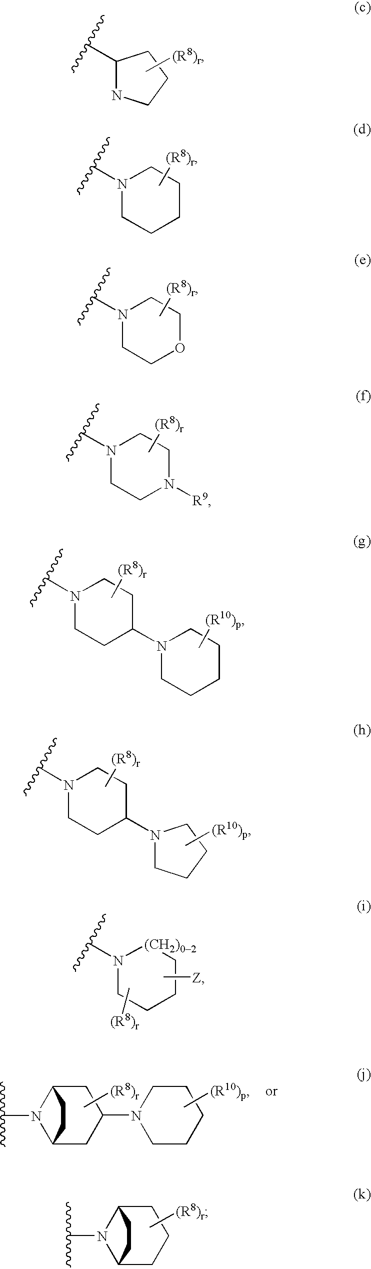

r is 2;

one R⁸ is —(C₁-C₆)alkyl, the second R⁸ is —O—(C₁-C₆)alkyl, both R groups are bonded to the same ring carbon atom, and together with the ring carbon atom to which they are attached, the two R⁸ groups define a spirocyclic ring.

In yet another embodiment of the compounds of formula I, Y is:

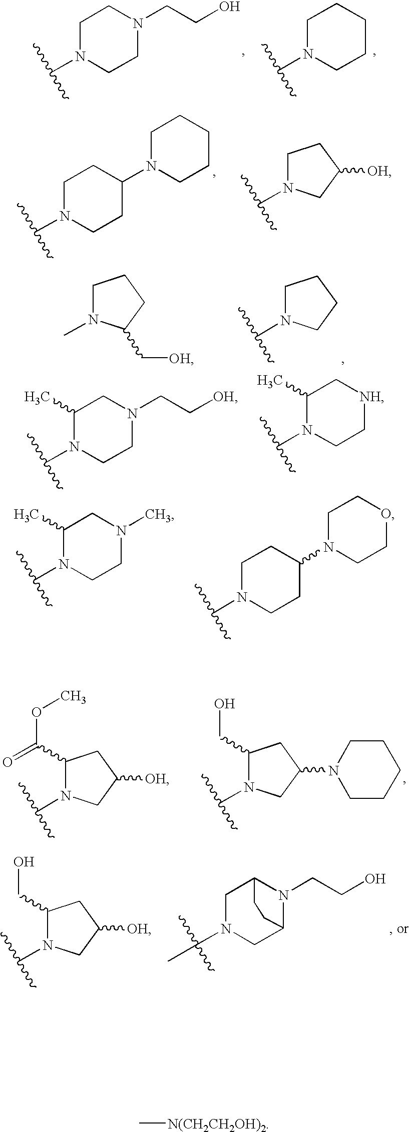

In yet another embodiment of the compounds of formula I, R⁶ and R⁷ are independently selected from the group consisting of H, methyl, ethyl, hydroxyethyl, —(C₃-C₈)cycloalkyl, -aryl(C₁-C₆)alkyl, 4-pyridylmethyl,

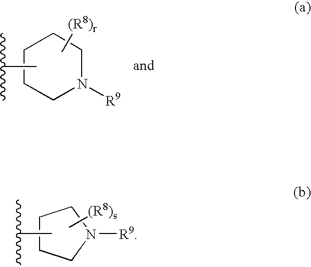

In yet another embodiment of the compounds of formula I, R⁸ is H, —OH, methyl, methoxy, ethoxy, —C(O)O—CH₃, —C(O)O—CH₂CH₃ or —(C₁-C₆)alkyl substituted with 1 to 4 —OH groups.

In yet another embodiment of the compounds of formula I, R⁸ is H, methyl, methoxy, hydroxyethyl or hydroxymethyl.

In yet another embodiment of the compounds of formula I, r is 2 and R⁸ is —OH and —C(O)O—(C₁-C₆)alkyl.

In yet another embodiment of the compounds of formula I, r is 2 and R⁸ is —OH and hydroxymethyl.

In yet another embodiment of the compounds of formula I, R⁸ is hydroxymethyl and Z is N-morpholinyl.

In yet another embodiment of the compounds of formula I, R⁸ is H and R⁹ is hydroxyethyl.

In yet another embodiment of the compounds of formula I, R⁸ is H and R⁹ is methyl.

In yet another embodiment of the compounds of formula I, at least one R⁸ is methyl and R⁹ is hydroxyethyl.

In yet another embodiment of the compounds of formula I, at least one R⁸ is methyl and R⁹ is methyl.

In yet another embodiment of the compounds of formula I, at least one R⁸ is methyl and R⁹ is H.

In yet another embodiment of the compounds of formula I, R⁹ is H, —(C₁-C₆)alkyl (e.g., methyl), —(C₁-C₆)alkyl substituted with 1 to 4 —OH groups (e.g., —(CH₂)₂OH), —(C₁-C₆)alkyl-O—(C₁-C₆)alkyl-OH (e.g., 2-(2-hydroxyethoxy)ethyl), (C₃-C₈)cycloalkyl, or heteroaryl, with the proviso that R⁹ is not hydroxymethyl.

In yet another embodiment of the compounds of formula I, R⁹ is H, methyl, cyclohexyl, 2-pyridyl, 2-hydroxyethyl or 2-(2-hydroxyethoxy)ethyl.

In yet another embodiment of the compounds of formula I, R¹⁰ is H or —(C₁-C₆)alkyl.

In yet another embodiment of the compounds of formula I, R¹⁰ is H or methyl.

In yet another embodiment of the compounds of formula I, R¹⁰ is H.

In yet another embodiment of the compounds of formula I, R¹¹ is selected from the group consisting of —(C₁-C₆)alkyl (e.g., methyl or ethyl), (C₃-C₈)-cycloalkyl (e.g., cyclopropyl), aryl (e.g., phenyl), aryl(C₁-C₆)alkyl (e.g., benzyl or —(CH₂)₂phenyl) and —(C₁-C₆)alkoxyalkyl (e.g., —CH₂OCH₃).

In still another embodiment, the compounds of formula I are represented by the following structural formulae:

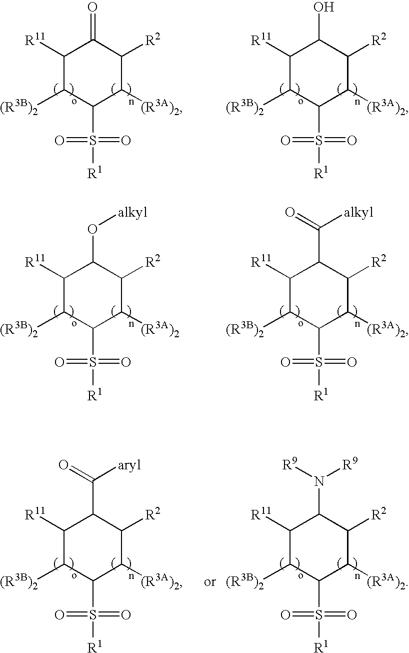

In still another embodiment, the compounds of formula I are represented by the following structural formulae:

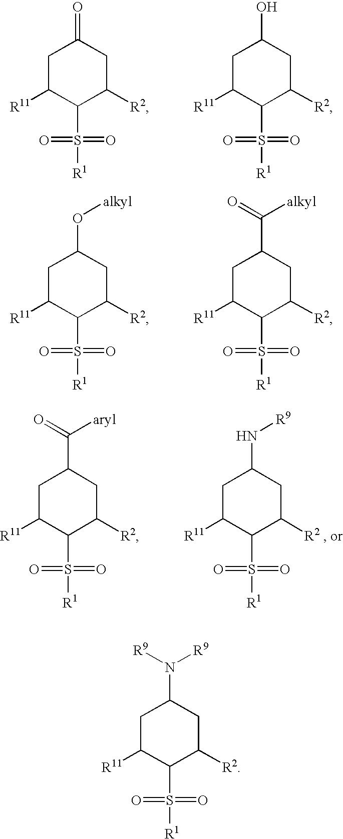

In still another embodiment, the compounds of formula I are selected from the group consisting of:

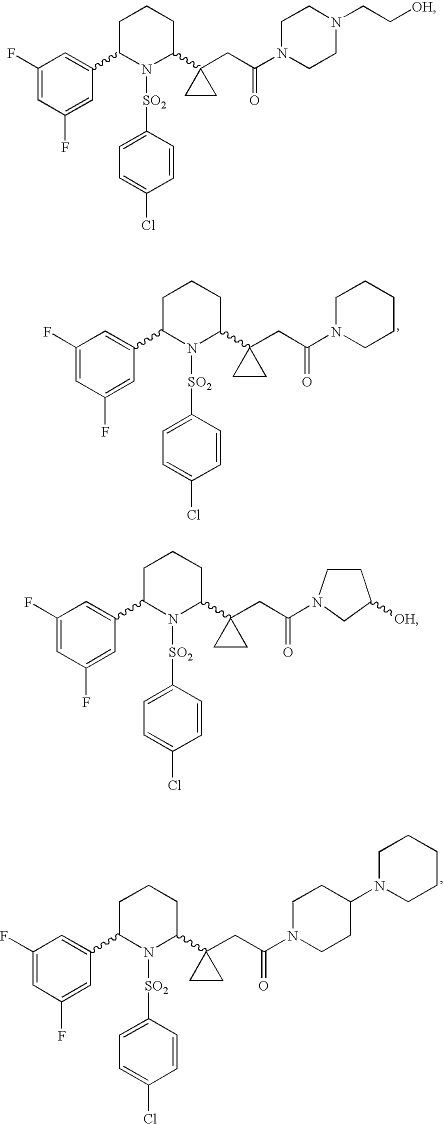

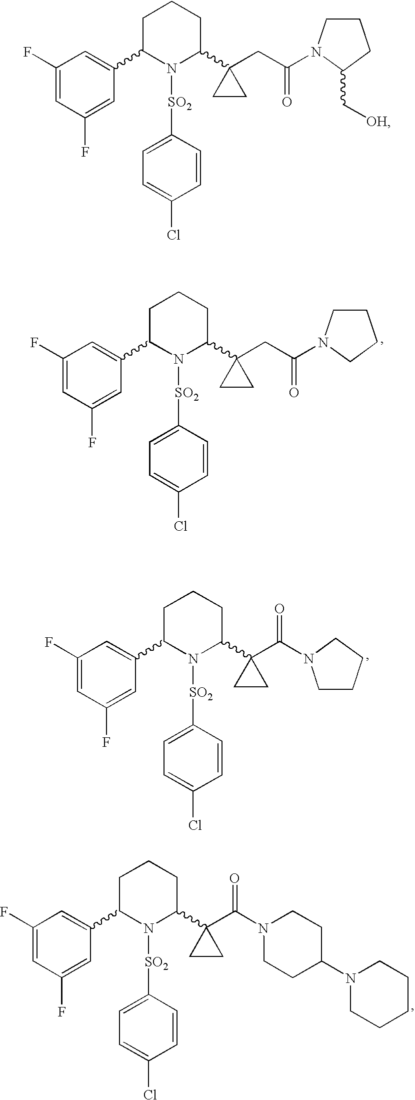

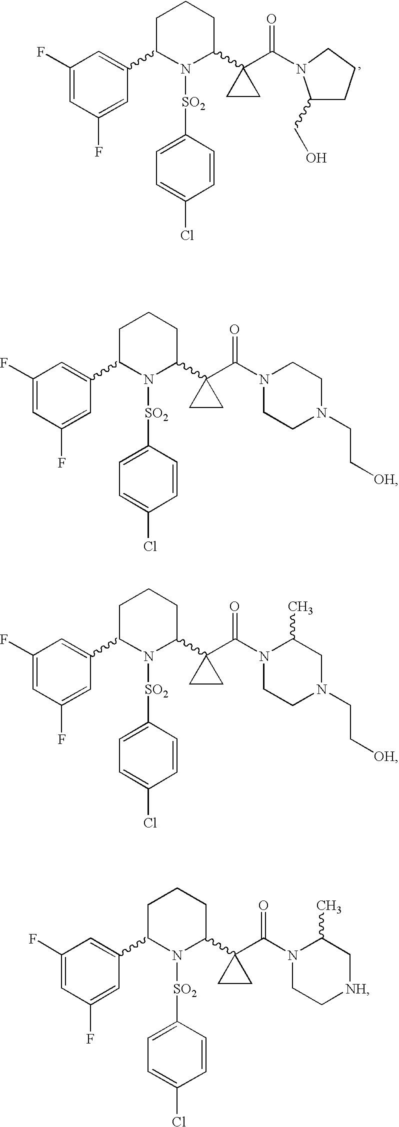

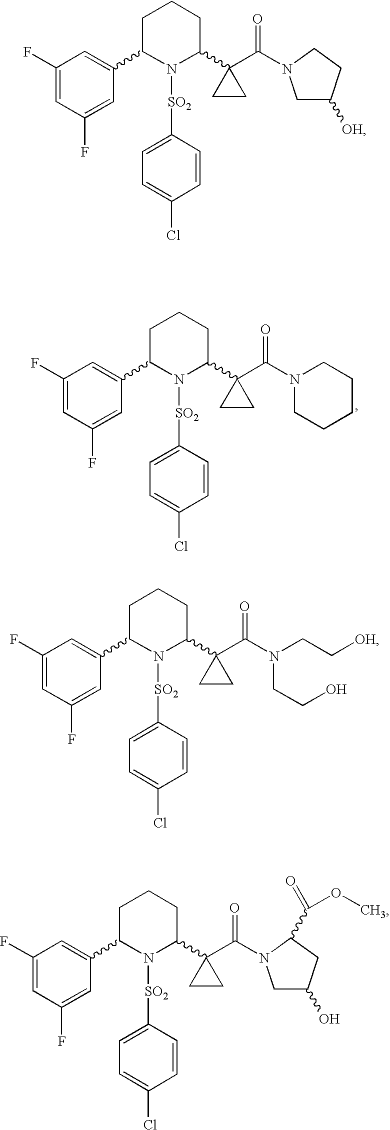

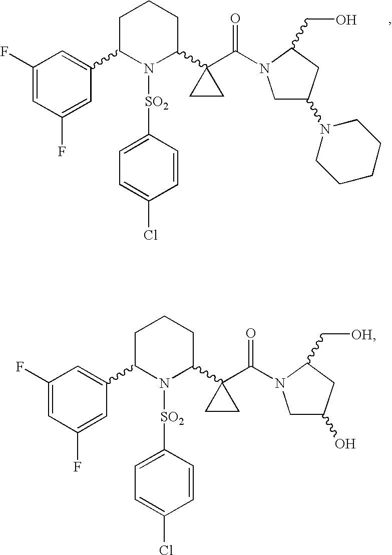

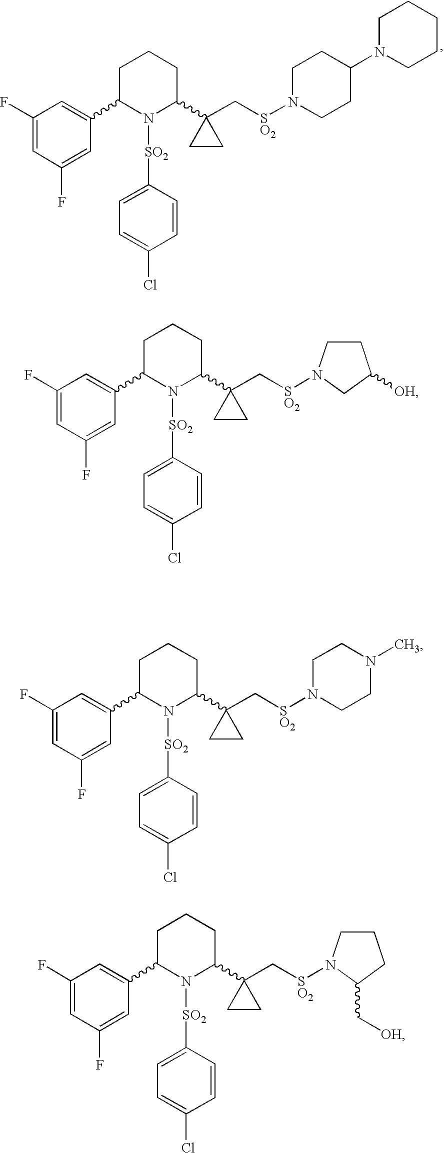

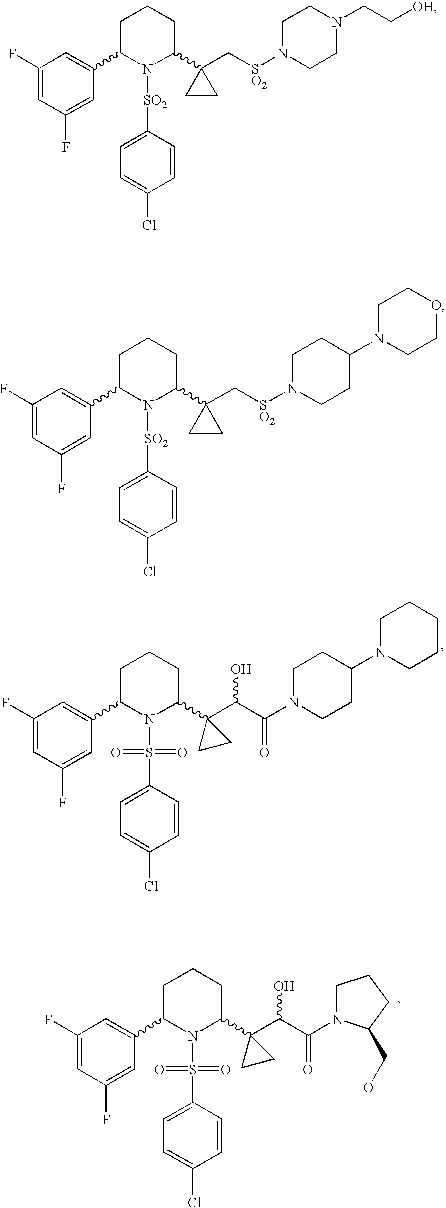

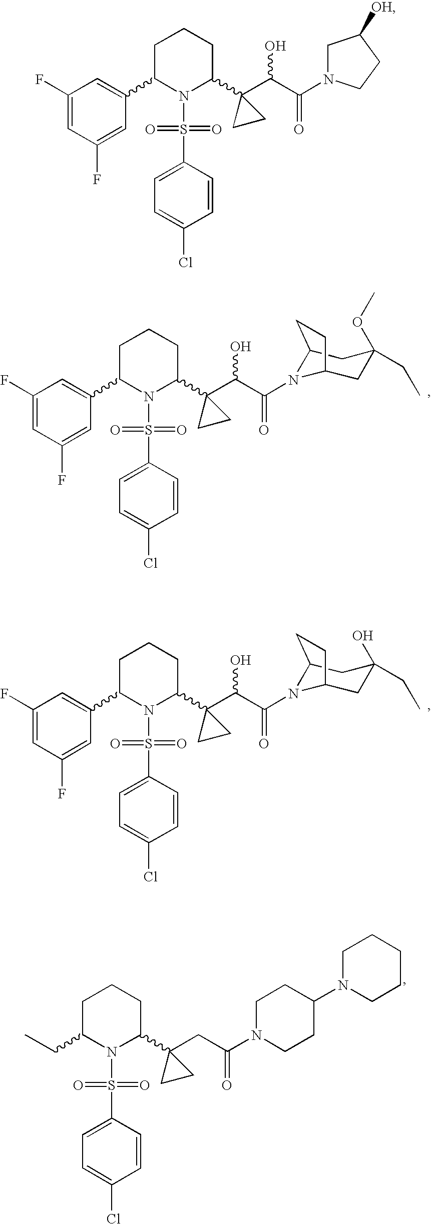

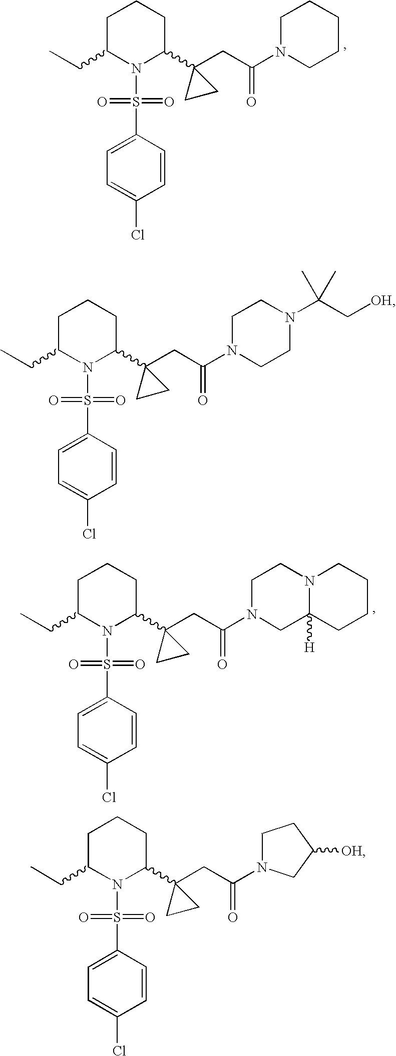

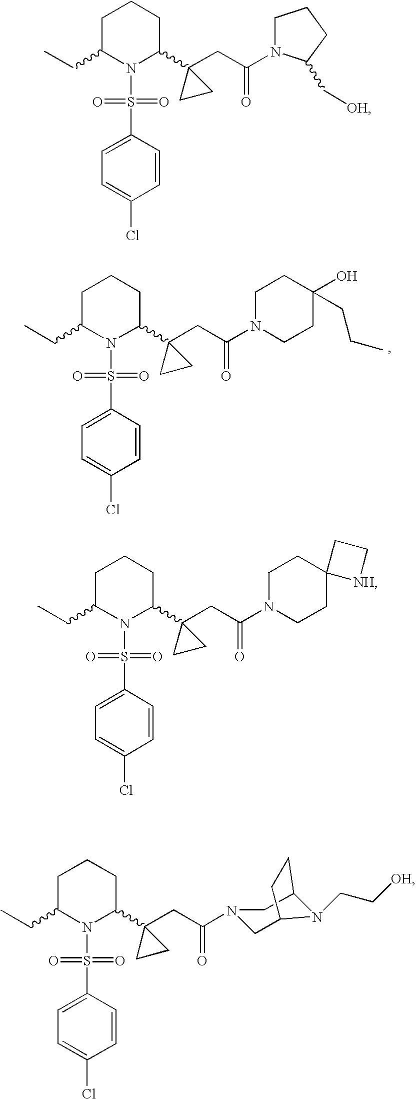

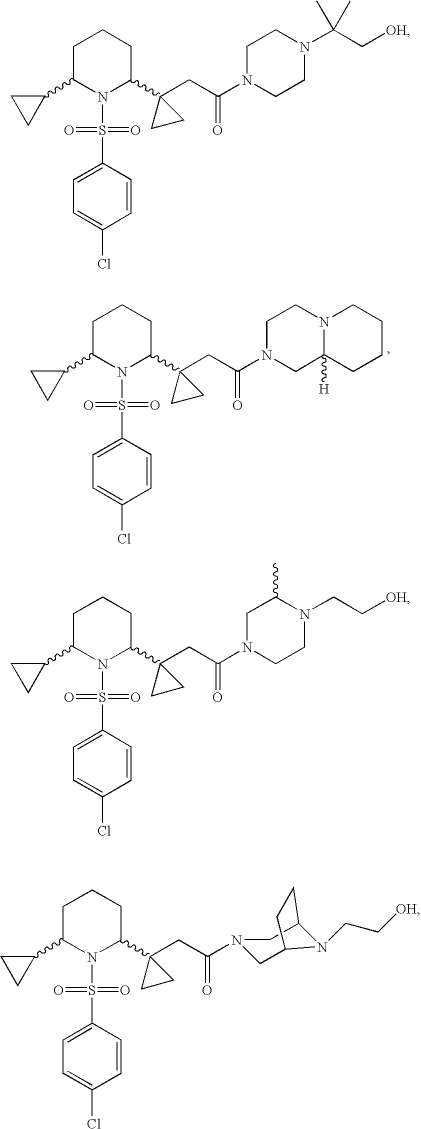

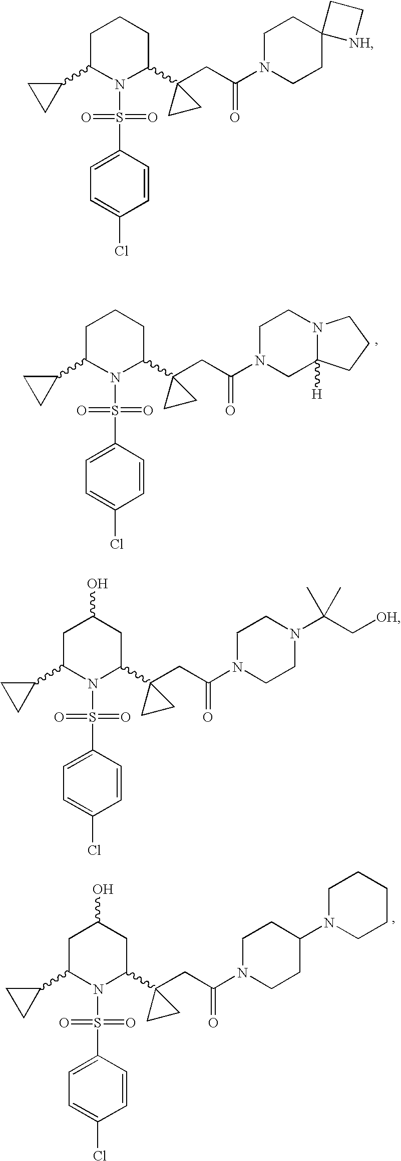

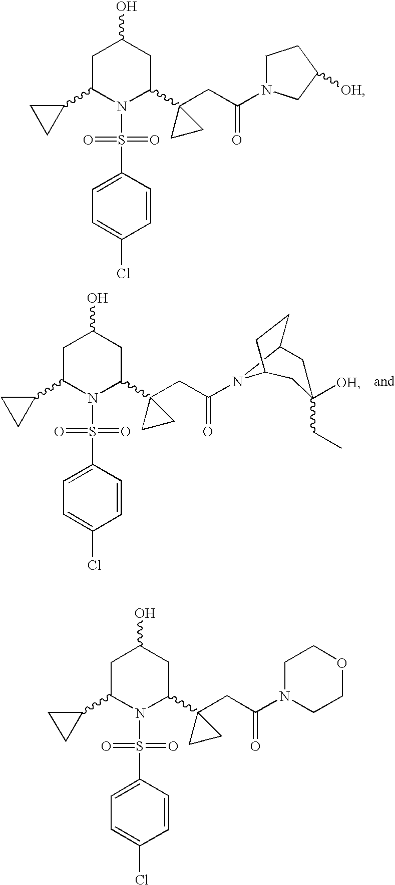

or stereoisomers pharmaceutically acceptable salts, solvates, and/or esters thereof.

In still another embodiment, the compounds of formula (I) are selected from the group consisting of:

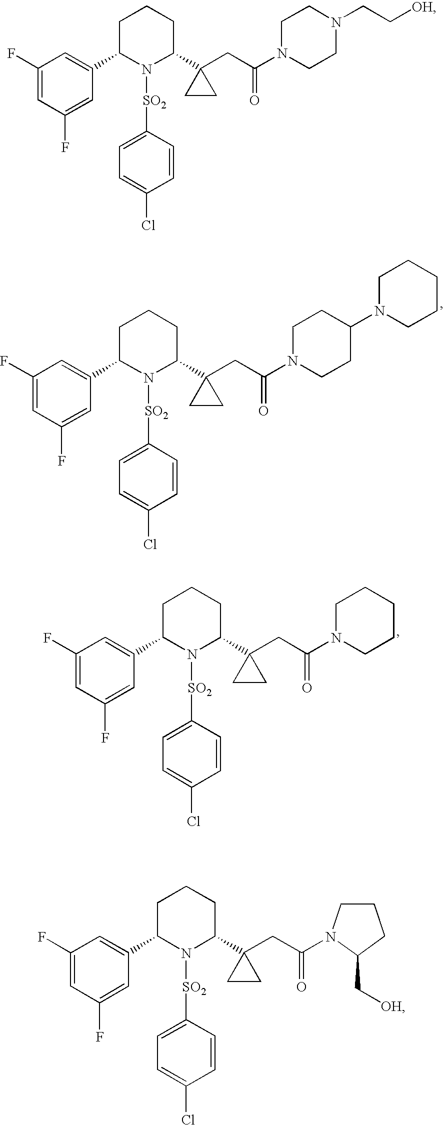

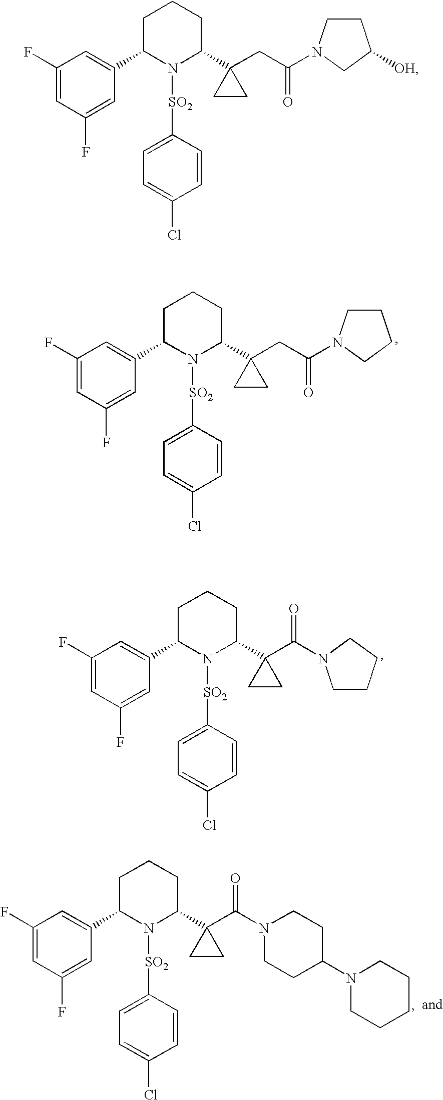

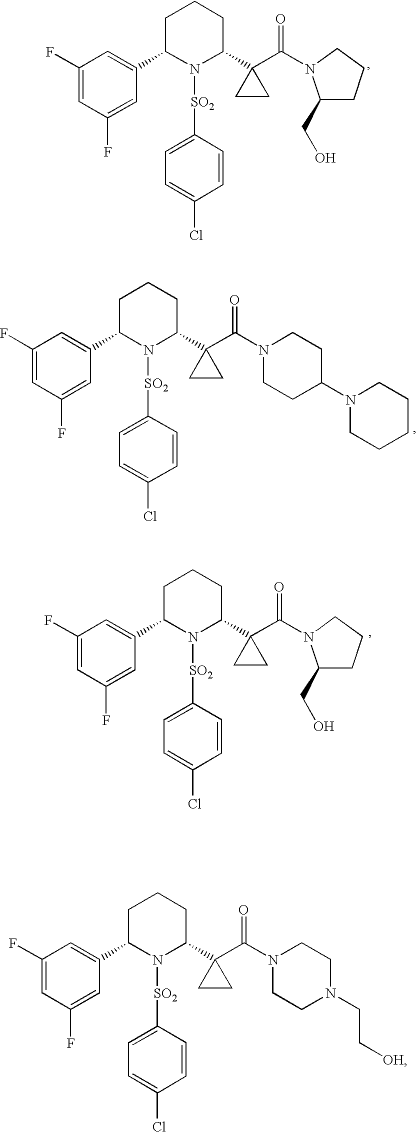

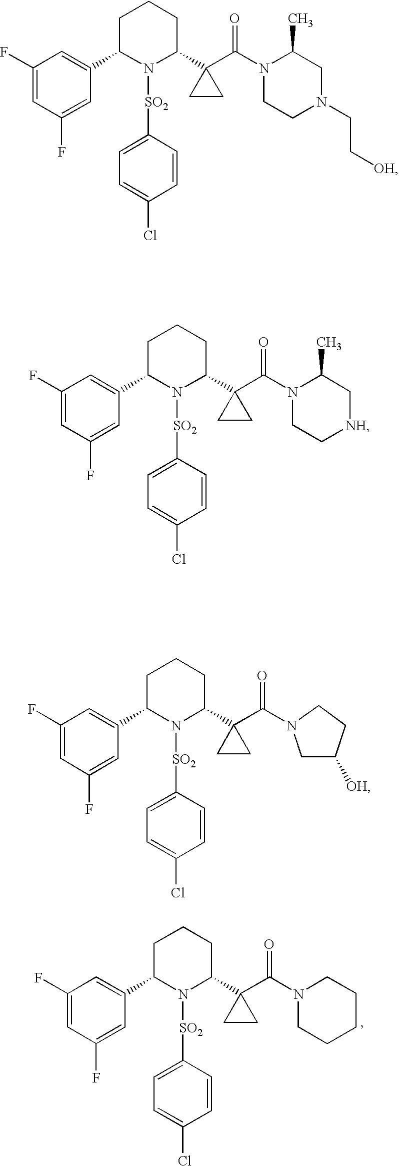

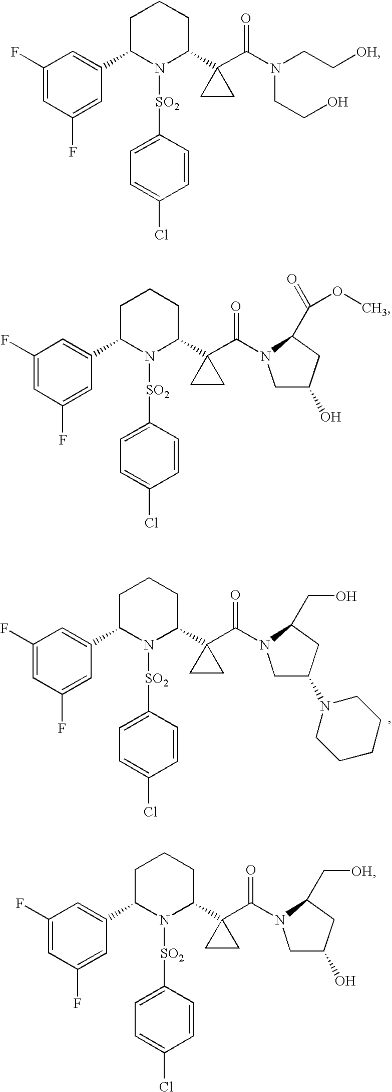

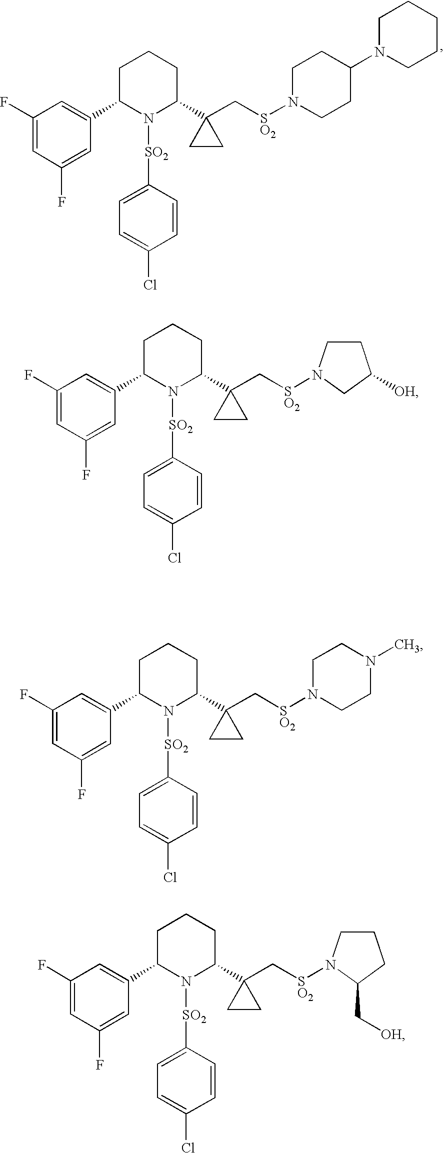

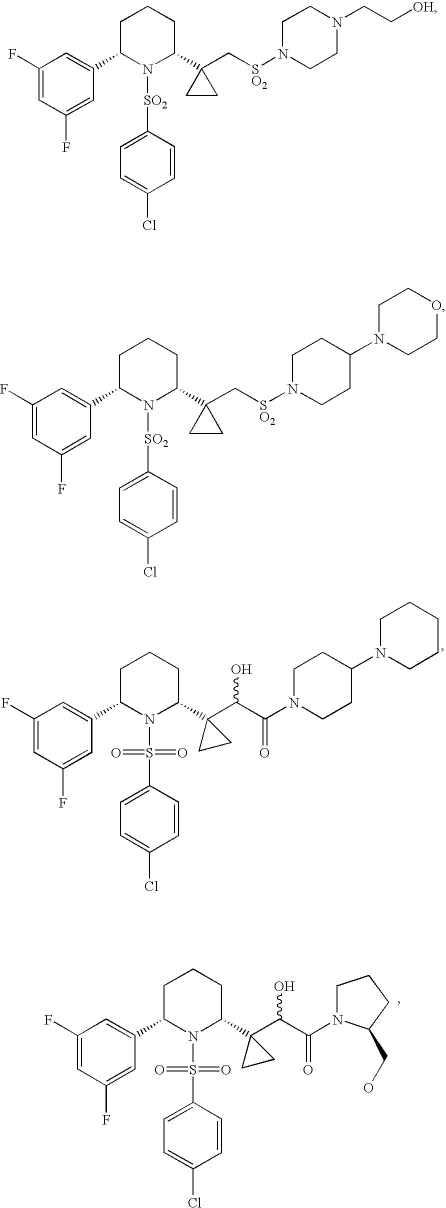

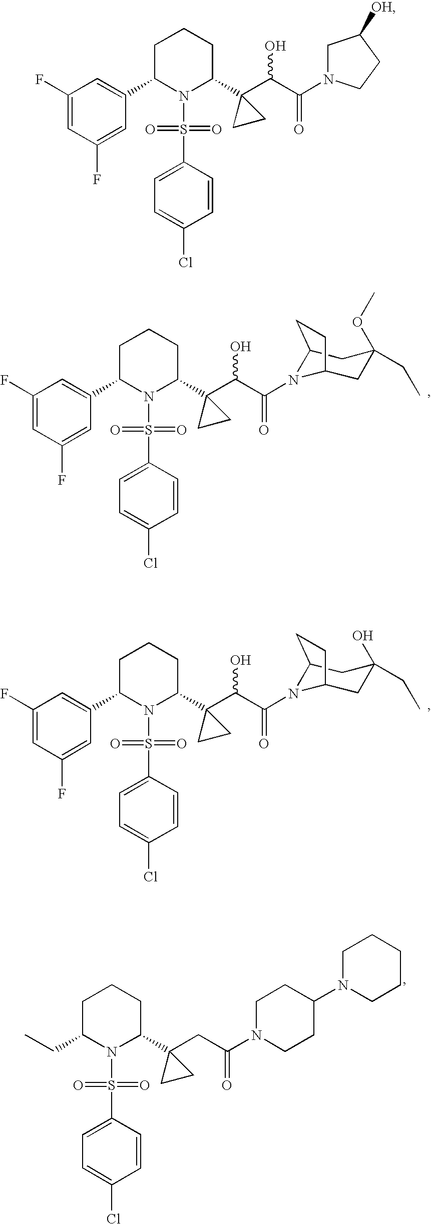

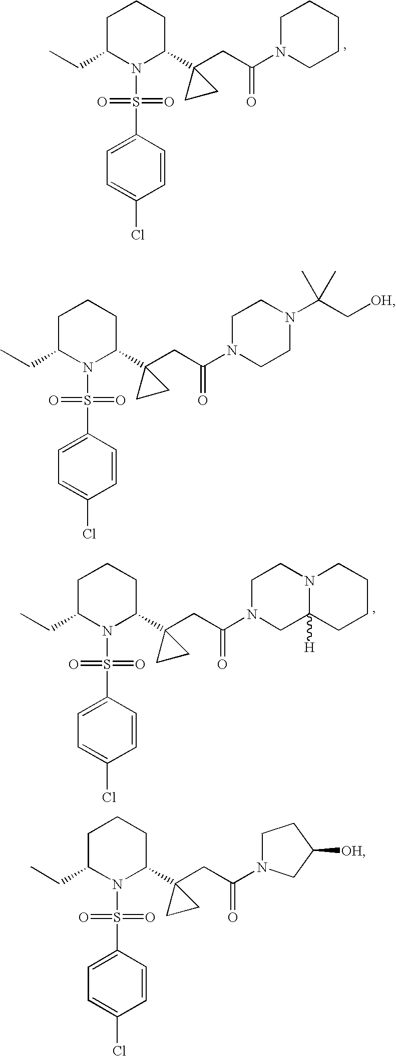

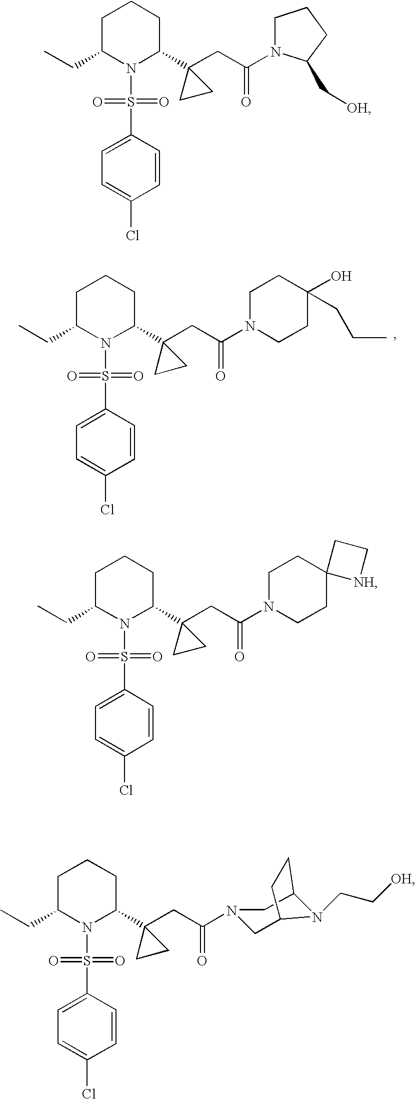

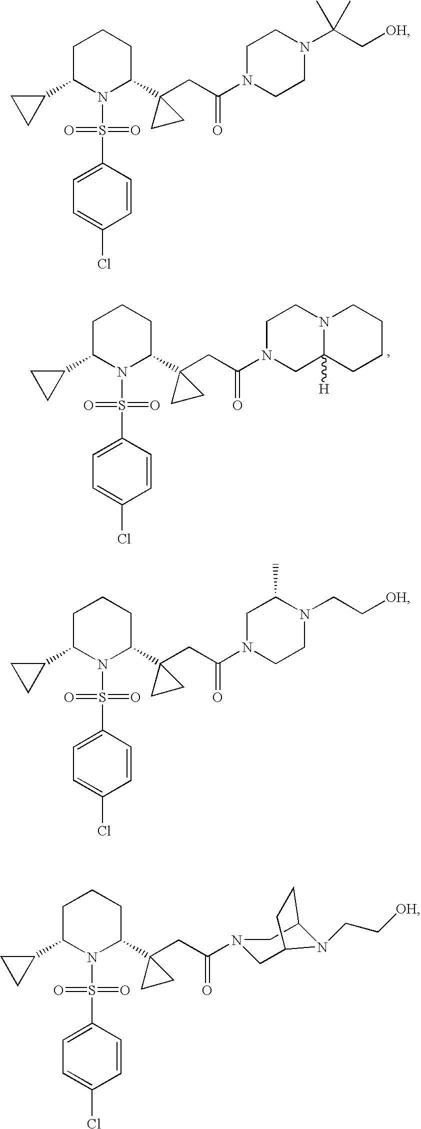

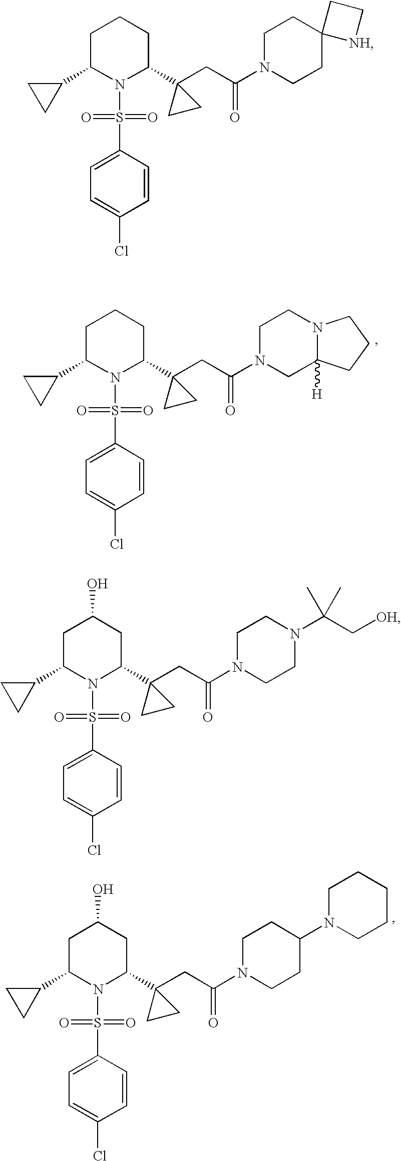

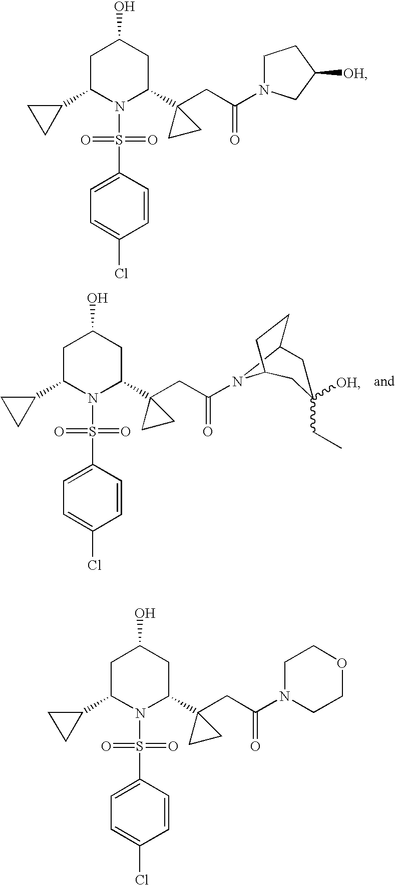

or pharmaceutically acceptable salts, solvates, and/or esters thereof.

Each reference to moieties preceded by an index, e.g., “m moieties”, refers to the moieties quantified by that index. Thus, for example, the term “m moieties” refers to the moieties whose quantity is indicated by the index “m”.

As used above, and throughout the specification, the following terms, unless otherwise indicated, shall be understood to have the following meanings:

“AcOH” means acetic acid.

“BOP” means benzotriazol-1-yloxy-tris(dimethylamino)-phosphonium hexafluorophosphate.

“cat.” means a catalytic amount.

“Cp” means cyclopentadienyl.

“DCE” means dichloroethane

“DCM” means dichloromethane.

“DIBAL” means diisobutylaluminum hydride.

“EDCI” means 1-(3-dimethylaminopropyl)-3-ethylcarbodiimide hydrochloride.

“Et” means ethyl.

“H₃⁺” means aqueous acid.

“HATU” means O-(7-azabenzotriazol-1-yl)-N,N,N′,N′-tetramethyluronium hexafluorophosphate.

“HOBT” means 1-hydroxybenzotriazole hydrate.

“LAH” means lithium aluminum hydride.

“LDA” means lithium diisopropylamide.

“MCPBA” means m-chloroperoxybenzoic acid.

“Me” means methyl.

“MsCl” means methanesulfonyl chloride.

“NMM” means N-methylmorpholine.

“t-Bu” means tert-butyl.

“Ph” means phenyl.

“TBSCl” means tert-butyidimethylsilyl chloride.

“TBSOTF” means tert-butyldimethylsilyltrifluromethanesulfonate.

“TBS” means t-butyldimethylsilyl.

“TBAF” means tetrabutylammonium fluoride.

“Tebbe reagent” means

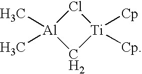

“TEMPO” means 2,2,6,6-tetramethyl-1-piperidinyloxy, free radical.

“Tf” means trifluoromethylsulfonyl.

“THF” means tetrahydrofuran.

“TLC” means thin layer chromatography.

“Ts” means toluene sulfonyl (also referred to as “tosyl”).

“Patient” includes both human and animals.

“Mammal” means humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy, —C(O)O-alkyl and —S(alkyl). Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain, and more preferably about 2 to about 4 carbon atoms in the chain. Branched means that one or more lower alkyl groups such as methyl, ethyl or propyl, are attached to a linear alkynyl chain.

“Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The term “substituted alkynyl” means that the alkynyl group may be substituted by one or more substituents which may be the same or different, each substituent being independently selected from the group consisting of alkyl, aryl and cycloalkyl.

“Alkylene” means a difunctional group obtained by removal of a hydrogen atom from an alkyl group that is defined above. Non-limiting examples of alkylene include methylene (i.e., —CH₂—), ethylene (i.e., —CH₂—CH₂— or —CH(CH₃)—) and propylene (i.e., —CH₂—CH₂—CH₂—, —CH(CH₂—CH₃)—, or —CH₂—CH(CH₃)—).

“Alkylene(OH)” means an alkylene as defined above, that is substituted with one or more —OH groups. Non-limiting examples of alkylene(OH) include —CH(OH)—, —CH₂CH(OH)—, etc.

“Aryl” (sometimes abbreviated “Ar”) means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thiophenyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thiophenopyridyl, quinazolinyl, thiophenopyrimidyl, pyrrolopyridyl, imidazopyridyl, isoquinolinyl, benzoazaindolyl, 1,2,4-triazinyl, benzothiazolyl and the like.

“Aralkyl” (or “arylalkyl”) means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting examples of suitable alkylaryl groups include o-tolyl, p-tolyl and xylyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like.

“Halo” means fluoro, chloro, bromo, or iodo groups. Preferred are fluoro, chloro or bromo, and more preferred are fluoro and chloro.

“Halogen” means fluorine, chlorine, bromine, or iodine. Preferred are fluorine, chlorine or bromine, and more preferred are fluorine and chlorine.

“Haloalkyl” means an alkyl as defined above wherein one or more hydrogen atoms on the alkyl is replaced by a halo group defined above.

“Ring system substituent” means a substituent attached to an aromatic or non-aromatic ring system which, for example, replaces an available hydrogen on the ring system. Ring system substituents may be the same or different, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocycloalkyl, heterocycloalkenyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl. “Ring system substituent” also means a cyclic ring of 3 to 7 ring atoms of which 1-2 may be a heteroatom, attached to an aryl, heteroaryl, heterocycloalkyl or heterocycloalkenyl ring by simultaneously substituting two ring hydrogen atoms on said aryl, heteroaryl, heterocycloalkyl or heterocycloalkenyl ring. Non-limiting examples include: 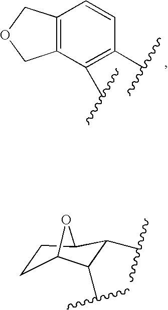
and the like.

“Cycloalkenyl” means a non-aromatic mono- or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Heterocycloalkenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination, and which contains at least one carbon-carbon double bond or carbon-nitrogen double bond. There are no adjacent oxygen and/or sulfur atoms present in the ring system. Preferred heterocycloalkenyl rings contain about 5 to about 6 ring atoms. The prefix aza, oxa or thia before the heterocycloalkenyl root name means that at least a nitrogen, oxygen or sulfur atom respectively is present as a ring atom. The heterocycloalkenyl can be optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of suitable monocyclic azaheterocycloalkenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Non-limiting examples of suitable oxaheterocycloalkenyl groups include 3,4-dihydro-2H-pyran, dihydrofuranyl, fluorodihydrofuranyl, and the like. Non-limiting example of a suitable multicyclic oxaheterocycloalkenyl group is 7-oxabicyclo[2.2.1]heptenyl. Non-limiting examples of suitable monocyclic thiaheterocycloalkenyl rings include dihydrothiophenyl, dihydrothiopyranyl, and the like.

“Heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocycloalkyls contain about 5 to about 6 ring atoms. The prefix aza, oxa or thia before the heterocycloalkyl root name means that at least a nitrogen, oxygen or sulfur atom respectively is present as a ring atom. The heterocycloalkyl can be optionally substituted by one or more “ring system substituents” which may be the same or different on the carbon(s) and/or heteroatoms(s), and are as defined herein. The nitrogen or sulfur atom of the heterocycloalkyl can be optionally oxidized to the corresponding N-oxide, S-oxide or S,S-dioxide. Non-limiting examples of suitable monocyclic heterocycloalkyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring: 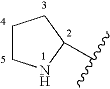
there is no —OH attached directly to carbons marked 2 and 5.

“Arylheterocycloalkyl” means a group derived from a fused aryl and heterocycloalkyl in which the aryl and heterocycloalkyl rings share two atoms, and the shared atoms in the rings may both be carbon, or when one or more of the heteroatoms are nitrogen, one or both shared atoms may be nitrogen. Non-limiting examples of suitable arylheterocycloalkyls include dihydrobenzofuran, dihydroisobenzofuran, dihydroindole and dihyroisoindole. The bond to the parent moiety is through the heterocycloalkyl ring.

“Arylcycloalkyl” means a group derived from a fused aryl and cycloalkyl in which the aryl and cycloalkyl rings have two carbon atoms in common. Preferred arylcycloalkyls are those wherein aryl is phenyl and the cycloalkyl consists of about 5 to about 6 ring atoms. The arylcycloalkyl can be optionally substituted by one or more ring system substituents, wherein “ring system substituent” is as defined above. Non-limiting examples of suitable arylcycloalkyls include 1,2,3,4-tetrahydronaphthyl, and the like. The bond to the parent moiety is through a non-aromatic carbon atom.

“Cycloalkylaryl” means a group derived from a fused arylcycloalkyl as described herein for an arylcycloalkyl group, except that the bond to the parent moiety is through an aromatic carbon atom.

“Heteroarylcycloalkyl” means a group derived from a fused heteroaryl and cycloalkyl as defined herein in which the heteroaryl and cycloalkyl rings have two carbon atoms in common. Preferred heteroarylcycloalkyls are those wherein the heteroaryl thereof consists of about 5 to about 6 ring atoms and the cycloalkyl consists of about 5 to about 6 ring atoms. The prefix aza, oxa or thia before heteroaryl means that at least a nitrogen, oxygen or sulfur atom is present respectively as a ring atom. The heteroarylcycloalkyl can be optionally substituted by one or more ring system substituents, wherein “ring system substituent” is as defined above. The nitrogen atom of the heteroaryl portion of the heteroarylcycloalkyl can be optionally oxidized to the corresponding N-oxide. Non-limiting examples of suitable heteroarylcycloalkyls include 5,6,7,8-tetrahydroquinolinyl, 5,6,7,8-tetrahydroisoquinolyl, 5,6,7,8- tetrahydroquinoxalinyl, 5,6,7,8-tetrahydroquinazolyl, 4,5,6,7-tetrahydro-1H-benzimidazolyl, 4,5,6,7-tetrahydrobenzoxazolyl, 1H-4-oxa-1,5-diazanaphthalen-2-onyl, 1,3-dihydroimidizole-[4,5]-pyridin-2-onyl, and the like. The bond to the parent moiety is through a non-aromatic carbon atom.

“Cycloalkylheteroaryl” means a group derived from a fused beteroarylcycloalkyl as described herein for heteroarylcycloalkyl, except that the bond to the parent moiety is through an aromatic carbon atom.

“Aralkenyl” means an aryl-alkenyl-group in which the aryl and alkenyl are as previously described. Preferred aralkenyls contain a lower alkenyl group. Non-limiting examples of suitable aralkenyl groups include 2-phenethenyl and 2-naphthylethenyl. The bond to the parent moiety is through the alkenyl.

“Aralkynyl” means an aryl-alkynyl-group in which the aryl and alkynyl are as previously described. Preferred aralkynyls contain a lower alkynyl group. The bond to the parent moiety is through the alkynyl. Non-limiting examples of suitable aralkynyl groups include phenacetylenyl and naphthylacetylenyl.

“Heteroaralkyl” (or “heteroarylalkyl”) means a heteroaryl-alkyl-group in which the heteroaryl and alkyl are as previously described. Preferred heteroaralkyls contain a lower alkyl group. Non-limiting examples of suitable aralkyl groups include pyridylmethyl, 2-(furan-3-yl)ethyl and quinolin-3-ylmethyl. The bond to the parent moiety is through the alkyl.

“Heteroaralkenyl” means a heteroaryl-alkenyl-group in which the heteroaryl and alkenyl are as previously described. Preferred heteroaralkenyls contain a lower alkenyl group. Non-limiting examples of suitable heteroaralkenyl groups include 2-(pyrid-3-yl)ethenyl and 2-(quinolin-3-yl)ethenyl. The bond to the parent moiety is through the alkenyl.

“Heteroaralkynyl” means a heteroaryl-alkynyl-group in which the heteroaryl and alkynyl are as previously described. Preferred heteroaralkynyls contain a lower alkynyl group. Non-limiting examples of suitable heteroaralkynyl groups include pyrid-3-ylacetylenyl and quinolin-3-ylacetylenyl. The bond to the parent moiety is through the alkynyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)—, alkenyl-C(O)—, alkynyl-C(O)—, cycloalkyl-C(O)—, cycloalkenyl-C(O)—, or cycloalkynyl-C(O)— group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, butanoyl and cyclohexanoyl.

“Aroyl” means an aryl-C(O)— group in which the aryl group is as previously described. The bond to the parent moiety is through the carbonyl. Non-limiting examples of suitable groups include benzoyl and 1- and 2-naphthoyl.

“Heteroaroyl” means a heteroaryl-C(O)— group in which the heteroaryl group is as previously described. Non-limiting examples of suitable groups include nicotinoyl and pyrrol-2-ylcarbonyl. The bond to the parent moiety is through the carbonyl.

“Alkoxy” means an alkyl-O— group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylamino” means an —NH₂ or —NH₃⁺ group in which one or more of the hydrogen atoms on the nitrogen is replaced by an alkyl group as defined above.

“Arylamino” means an —NH₂ or —NH₃⁺ group in which one or more of the hydrogen atoms on the nitrogen is replaced by an aryl group as defined above.

“Alkylthio” means an alkyl-S— group in which the alkyl group is as previously described. Non-limiting examples of suitable alkylthio groups include methylthio, ethylthio, i-propylthio and heptylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—C(O)— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Alkylsulfinyl” means an alkyl-S(O)— group. Preferred groups are those in which the alkyl group is lower alkyl. The bond to the parent moiety is through the sulfinyl.

“Arylsulfonyl” means an aryl-S(O₂)— group. The bond to the parent moiety is through the sulfonyl.

“Arylsulfinyl” means an aryl-S(O)— group. The bond to the parent moiety is through the sulfinyl.

The term “cycloalkylene” refers to substitution on the same carbon atom in an alkylene group with a cyclic group. Nonlimiting examples include

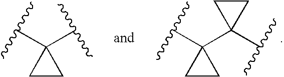

It should also be noted that any heteroatom with unsatisfied valences in the text, schemes, examples and Tables herein is assumed to be attached to a sufficient number of hydrogen atoms to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York, incorporated herein by reference in its entirety.

When any variable (e.g., aryl, heterocycle, R³, etc.) occurs more than one time in any constituent or in formula I, its definition on each occurrence is independent of its definition at every other occurrence.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

As used herein, the term “composition” is intended to encompass a product comprising the specified ingredients in the specified amounts, as well as any product which results, directly or indirectly, from combination of the specified ingredients in the specified amounts.

The wavy line [symbol] as a bond generally indicates a mixture of, or either of, the possible isomers, e.g., containing (R)- and (S)-stereochemistry. For example, 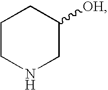
means containing both

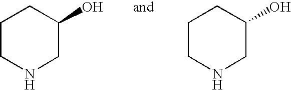

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in inhibiting gamma-secretase and thus producing the desired therapeutic effect in a suitable patient.

The compounds of formula I form salts that are also within the scope of this invention. Reference to a compound of formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formula I may be formed, for example, by reacting a compound of formula I with an amount of acid or base, such as an equivalent amount, in a medium such as one in which the salt precipitates or in an aqueous medium followed by lyophilization.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, Intemational J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of the invention with a carboxylic acid group can form pharmaceutically acceptable esters with an alcohol. Examples of suitable alcohols include methanol and ethanol.

Likewise, compounds of the invention with a hydroxyl group can form pharmaceutically acceptable esters with a carboxylic acid, e.g., acetic acid.

Compounds of formula I, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Polymorphic forms of the compounds of formula I, and of the salts, solvates and/or prodrugs of the compounds of formula I, are also intended to be included in the present invention.

Any formula, compound, moiety or chemical illustration with otherwise unsatisfied valences in the present specification and/or claims herein is assumed to have the requisite number of hydrogen atoms to satisfy the valences.

The compounds according to the invention have pharmacological properties; in particular, the compounds of formula I can be used for the treatment or prevention of neurodegenerative diseases, such as Alzheimer's Disease, and other diseases relating to the deposition of amyloid protein.

Those skilled in the art will appreciate that the term “neurodegenerative disease” has its commonly accepted medical meaning and describes diseases and conditions resulting from abnormal function of neurons, including neuronal death and abnormal release of neurotransmitters or neurotoxic substances. In this instance it also includes all diseases resulting from abnormal levels of beta amyloid protein. Examples of such diseases include, but are not limited to, Alzheimer's disease, age-related dementia, cerebral or systemic amyloidosis, hereditary cerebral hemorrhage with amyloidosis, and Down's syndrome.

Lines drawn into the ring systems, such as, for example: 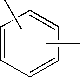
indicate that the indicated line (bond) may be attached to any of the substitutable ring carbon atoms.

As well known in the art, a bond drawn from a particular atom wherein no moiety is depicted at the terminal end of the bond indicates a methyl group bound through that bond to the atom. For example:

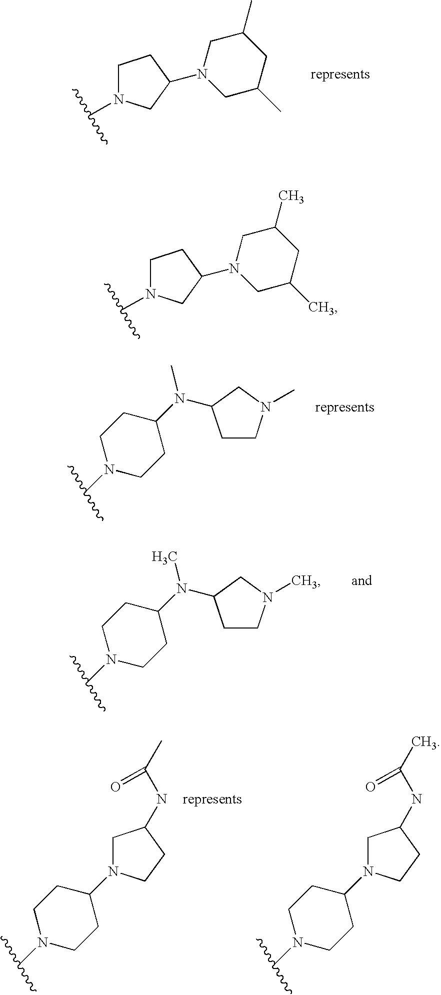

Compounds of formula I can be prepared by various methods well known to those skilled in the art, and by the methods described below.

Pharmaceutical compositions can comprise one or more of the compounds of formula I. For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active compound. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa., herein incorporated by reference.

Liquid form preparations include solutions, suspensions and emulsions. Water or water-propylene glycol solutions may be mentioned as examples for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The pharmaceutical preparation may also be formulated in a unit dosage form. In such form, the preparation is subdivided into suitably sized unit doses containing appropriate quantities of the active compound, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in one to four divided doses.

Representative compounds of the invention include, but are not limited to, the compounds of Examples 1-24.

The compounds of formula I can be prepared by various methods well known to those skilled in the art, and by the methods described below.

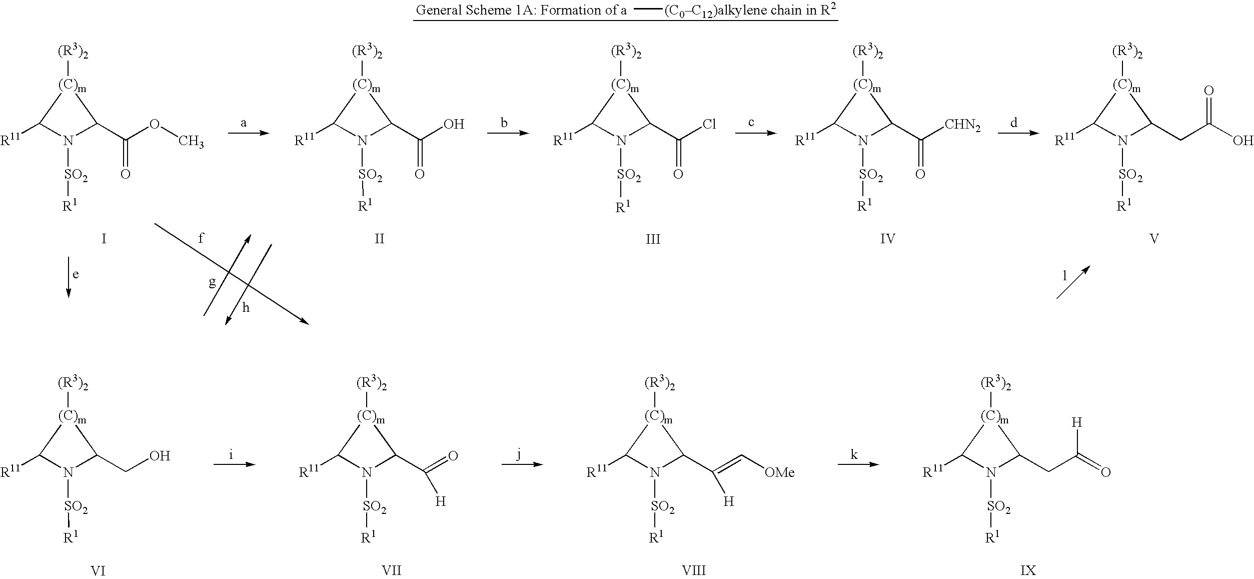

Reaction Steps
(a) NaOH/H₂O/EtOH or THF/LiOH/H₂O
(b) (C(O)Cl)₂/DCM/DMF (cat.) or SOCl₂/solvent
(c) diazomethane
(d) Ag⁺/H₂O/organic co-solvent
(e) LAH
(f) DIBAL
(g) RuCl₃ (cat.)/NalO₄
(h) Diborane
(i) Dess-Martin or Swern oxidation conditions (for example as described in Dess, D. B., Martin, J. C., J. Org. Chem., 1983, vol. 48, beginning at p. 4155; Omura, K., Swern, D. Tetrahedron, 1978, vol. 34, beginning at p. 1651; both references are herein incorporated by reference in their entirety)
(j) Methoxymethyltriphenylphosphonium bromide (or chloride)/base
(k) H₃O⁺
(l) NaClO₂

Ester I may be prepared, for example, by the methods described in U.S. Ser. No. 10/358,898 (compound 19); said application is herein incorporated by reference in its entirety. Compound I can be converted to carboxylic acid II by direct hydrolysis (i.e., step (a)) or by a two step procedure in which ester I is reduced to alcohol VI in step (e), followed by oxidation of VI in step (g). The carboxylic acid side chain of compound II may be homologated via intermediates III and IV using the Arndt-Eistert synthesis, e.g., as described in W. E. Bachmann, Org. React. 1, 38-39,1942 (which is herein incorporated in its entirety), thereby providing homologated carboxylic acid V. The carboxylic acid side chain of compound V can be further homologated by repeating the Arndt-Eistert synthesis steps (i.e., using compound V as the starting material, then successively applying steps (b), (c), and (d)). By repeating the Arndt-Eistert synthesis steps in this manner, alkylene chains of any desired length may be prepared (e.g., as in General Scheme 1 B, below).

Alternatively, the homologation may be carried out by preparing aldehyde VII, either by reduction of compound I with DIBAL (i.e., step (f)) or by oxidation of alcohol VI in step (i). Alcohol VI may be prepared by reducing compound I in step (e) or by reducing compound II in step (h). Aldehyde VII can then be reacted under Wittig reaction conditions (e.g., step (j))) to enol ether VIII, which in turn can be hydrolyzed to aldehyde IX (e.g., step (k)).

In an alternative synthesis of aldehyde IX, alcohol VI can be first converted to an iodide, for example by a combination of triphenylphosphine with iodine (General Scheme 1Aa). Subsequent displacement of iodide with cyanide and reduction of the resulting nitrile with DIBAL can furnish aldehyde IX. General Scheme 1Aa: Alternate Synthesis of Aldehyde IX 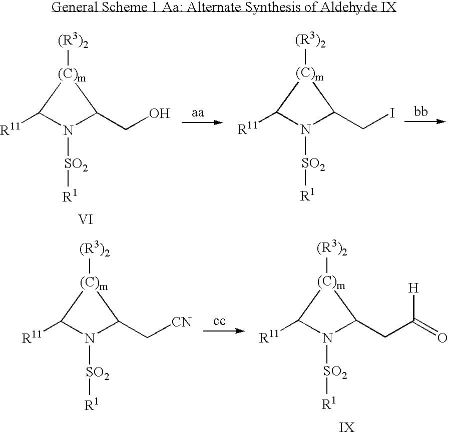
Reaction Steps
(aa) PPh₃/I₂
(bb) n-Bu₄NCN
(cc) DIBAL

The same cycle of homologation using Wittig reaction conditions can be repeated starting with aldehyde IX, or aldehyde IX can be oxidized to the corresponding acid V and further homologated by repeating the Arnd-Eistert synthesis steps as discussed above, thereby providing compound X as in Scheme 1B, below.

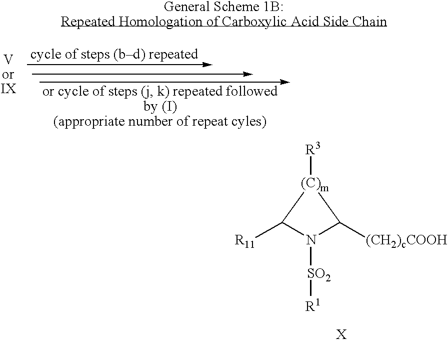

Of course, starting material I of General Scheme 1A is only one of many possible starting materials which may be used to prepare compounds according to formula I. For example, the homologation reaction conditions described in General Schemes 1A and 1B are not limited to starting materials in which indices n and o are both 0 (e.g., compound 1). 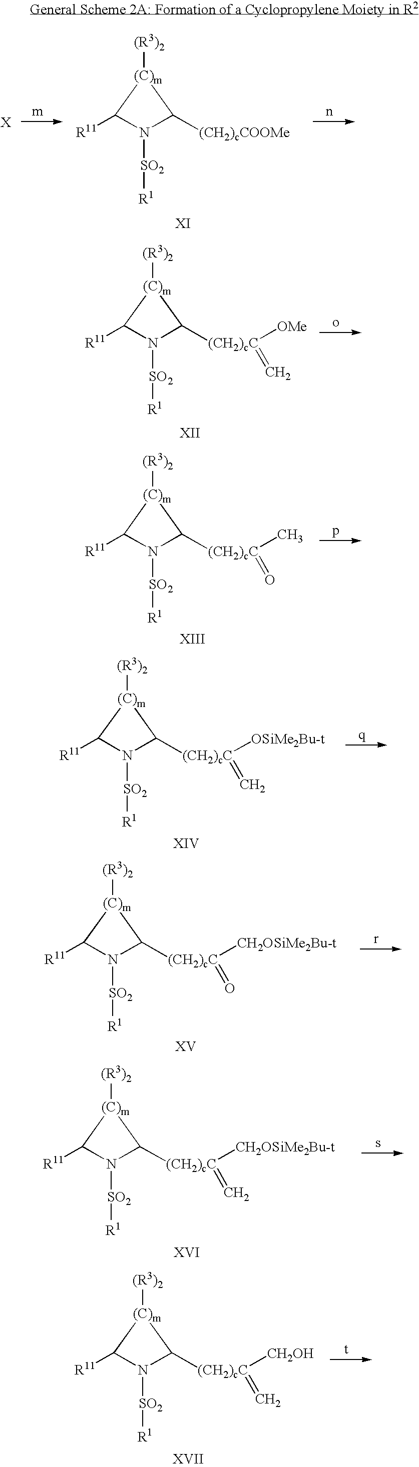 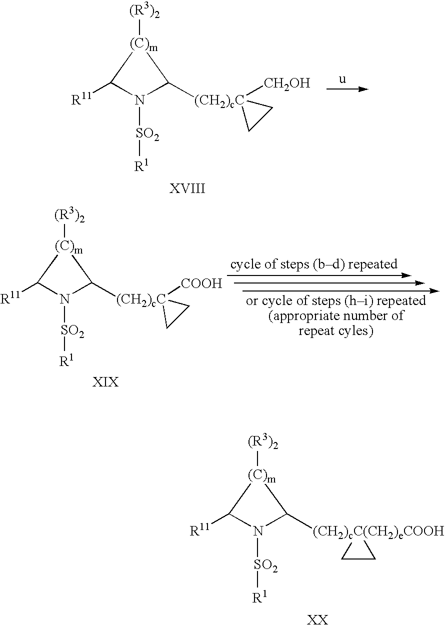
Reaction Steps
(m) SOCl₂/MeOH
(n) Tebbe reagent
(o) H₃O⁺
(p) t-BuSiMe₂-OS(O₂)CF₃/Et₃N
(q) MCPBA
(r) Ph₃CH₃P⁺Cl⁻/BuLi
(s) Tetrabutylammonium fluoride
(t) Et₂Zn/lCH₂Cl
(u) RuCl₃/NalO₄

Carboxylic acid X (e.g., prepared according to General Scheme 1B) is converted to methyl ester XI in step (m), and the methyl ester may then be converted to allylic alcohol XVII by a number of known methods. For example, methyl ester XI may be converted to enol ester XII by olefination with Tebbe reagent in step (n) (S. H. Pine et al, Org. Synth., 69, 72-79, 1990, herein incorporated by reference in its entirety), followed by hydrolytic conversion to ketone XIII in step (o). Ketone XIII is then converted into silyl enol ether XIV in step (p), and is oxidized in step (q) (N. Yamamoto, M. Isobe, Tetrahedron 1993, 49 (30), 6581-6590, herein incorporated by reference) to form t-butyldimethylsilyloxy ketone XV. Wittig olefination of ketone XV in step (r) provides compound XVI. Cleavage of the silyl protecting group of compound XVI in step (s) provides allylic alcohol XVII, which is cyclopropanated in step (t) to provide alcohol XVIII. Alcohol XVIII is then oxidized in step (u) to carboxylic acid XIX. Further homologation of the carboxylic acid can be carried out, if desired, as discussed above in General Scheme 1B, to provide compound XX. 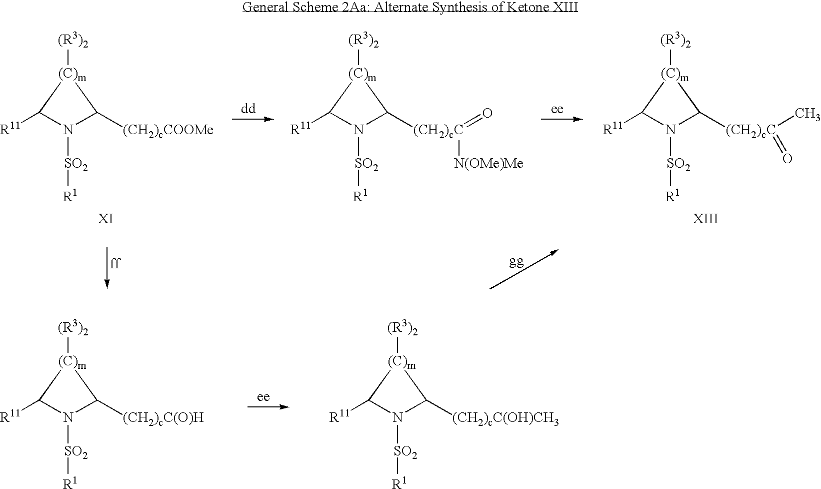
Reaction Steps
(dd) HN(OMe)Me/i-PrMgCl
(ee) MeMgBr
(ff) DIBAL
(gg) Dess-Martin periodinane

Ester XI can be converted to an N-methyl-N-methoxyamide in step (dd), which can further react with methyl Grignard reagent (ee) to furnish ketone XIII. Alternatively, ester XI can be converted to an aldehyde, for example by reduction with DIBAL. Reaction of the aldehyde with methyl Grignard reagent can provide a secondary alcohol, which can be oxidized to ketone XIII in step (gg). 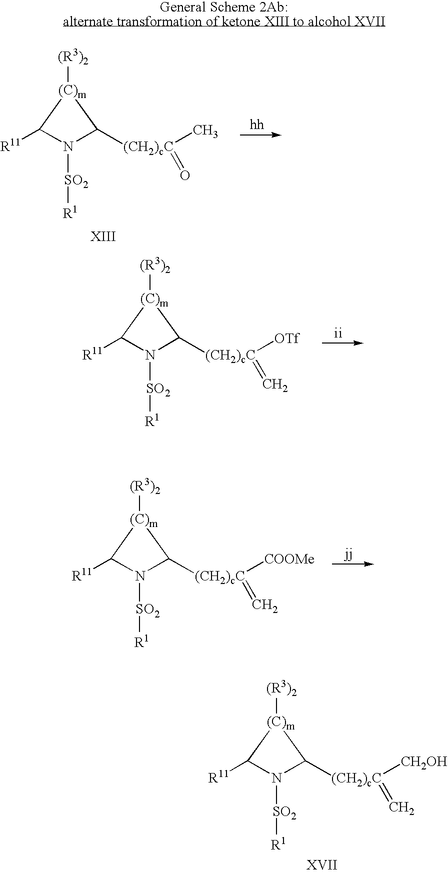
Reaction Steps
(hh) LDA/2-[N,N-bis(trifluoromethylsulfonyl)amino]-5-chloropyridine
(ii) MeOH/CO/Pd(PPh')₄ (cat)
(jj) DIBAL

Ketone XIII can be converted to an enol triflate in step (hh). The enol triflate can then be carbonylated using carbon monoxide to furnish a conjugated ester in step (ii). Reduction of the ester in step (jj), for example with an excess of DIBAL, provides alcohol XVII.

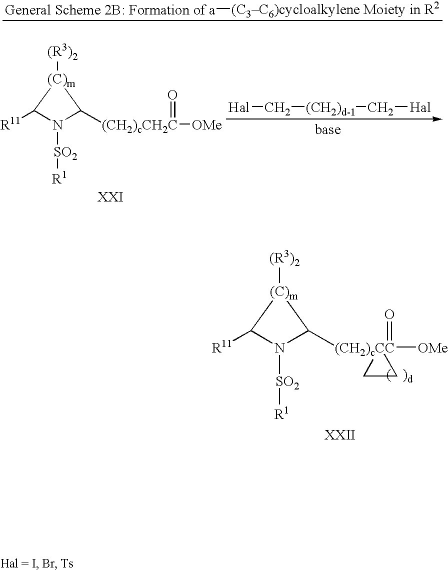

Cycloalkylene moieties other than cyclopropyl may be formed, for example by the method of General Scheme 2B, when the R² side chain has a carbonyl group situated next to a methylene group. For example, compound XXI may be reacted with a bis-halide or bis-tosylate in the presence of a suitable base to form the cycloalkylene ketone XXII. Those skilled in the art will recognize that XXI is a simply a special case of XI, wherein c is at least 1.

Likewise, starting materials XX and XXI of General Schemes 2A and 2B, respectively, are not the only possible starting materials which may be used to prepare compounds according to formula I in General Schemes 2A and 2B. For example, the cyclization reaction conditions described in General Schemes 2A and 2B are not limited to starting materials in which indices n and o are both 0 (e.g., compounds XX and XXI).

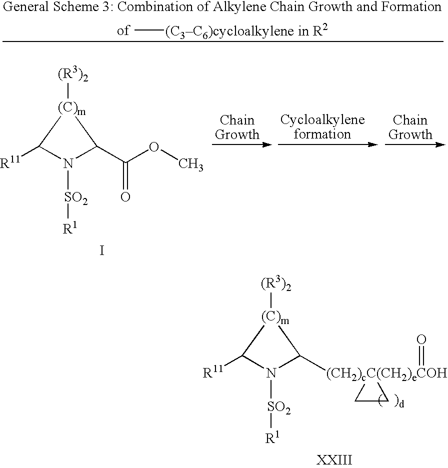

Those of skill in the art will recognize that alkylene chain growth procedures (e.g., General Schemes 1A and 1B) and cycloalkylene forming procedures (e.g., General Schemes 2A and 2B) may be combined in various ways to provide various combinations of alkylene and cycloalkylene moieties on the R² side chain of compounds according to formula I. For example, as shown in General Scheme 3, compound I may be homologated to extend the alkylene chain to the extent desired, then a cycloalkylene moiety may be formed, followed, if desired, by additional homologation of the alkylene, to provide compound XXIII.

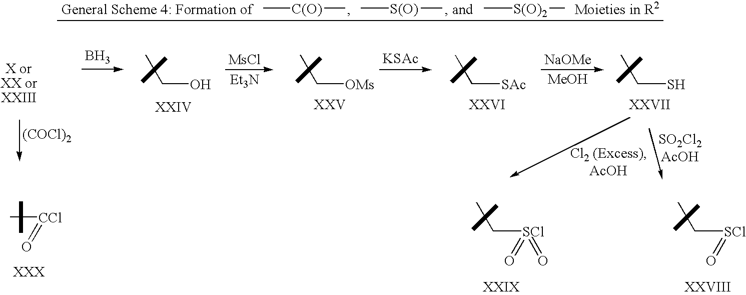

Carboxylic acids X or XX or XXIII can be reduced to the corresponding alcohol XXIV by reaction with borane. The alcohol XXIV can then be reacted with a suitable reagent, such as mesyl chloride (i.e., methane sulfonyl chloride) and triethylamine, to form a compound having a suitable leaving group, e.g., mesylate XXV. The mesylate group can then be displaced with potassium thioacetate to provide thioacetic ester XXVI, which after hydrolysis (e.g., sodium methoxide in methanol) provides thiol XXVII. Oxidation of thiol XXVII with sulfuryl chloride provides sulfinyl chloride XXVIII (Youn, J.-H.; Herrmann, R.; Synthesis 1987 (1), 72, herein incorporated by reference in its entirety). Oxidation of thiol XXVII with excess chlorine provides sulfonyl chloride XXIX (Barnard, D.; Percy, E. J.; J Chem Soc 1962, 1667, herein incorporated by reference in its entirety). Alternatively, the reaction of carboxylic acids X or XX or XXIII with oxalyl chloride (optionally with catalytic DMF present) provides acyl chloride XXX.

Those of skill in the art will recognize that the reactions described in General Scheme 4, above, are not limited to the specific starting materials shown, but may be carried out with other carboxylic acid compounds.

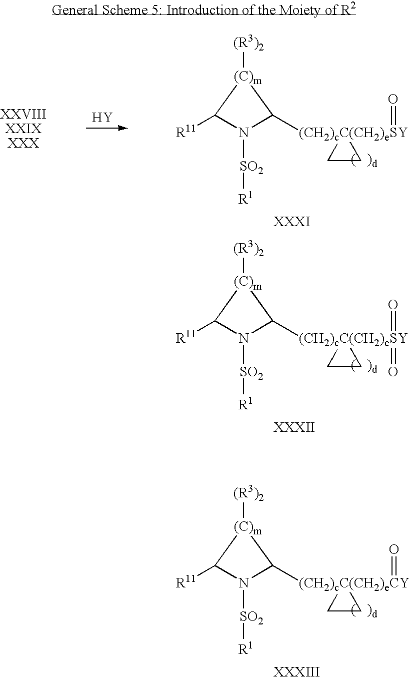

The moiety Y of R² may be introduced by reaction of the appropriate sulfinyl chloride, sulfonyl chloride, or acyl chloride (e.g., prepared as described in Scheme 4) with the appropriate HY, optionally in the presence of an organic base such as triethylamine. For example, compounds XXVIII, XXIX, and XXX may be reacted with HY (e.g., wherein HY is piperidine, pyrrolidine, substituted piperidine, substituted pyrrolidine, etc.) to form compounds XXXI, XXXII, and XXXIII according to formula I. Compound XXXIII may also be prepared by the coupling of carboxylic acids X, XX, or XXIII with HY using amide forming conditions, for example the conditions described in Humphrey, J. M., Chamberlin, R., Chem. Rev., 1997, vol. 97, pp. 2243-2266, herein incorporated by reference in its entirety.

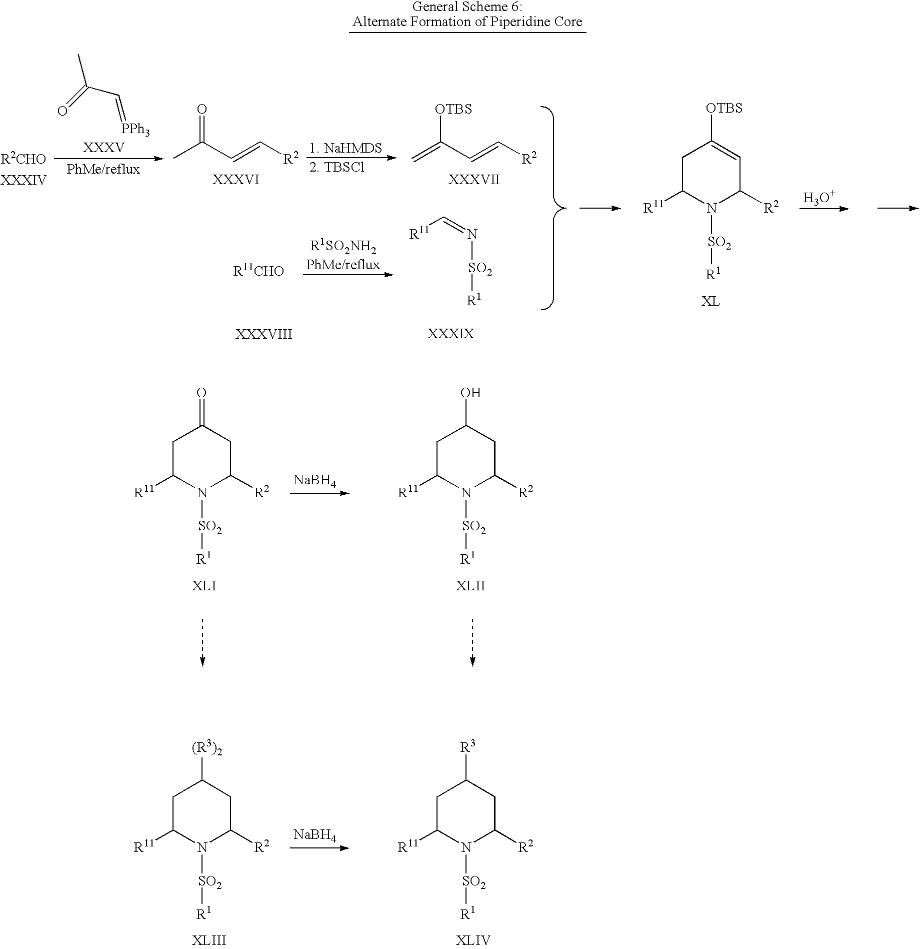

Alternatively, the piperidine “core” of the compounds of the present invention can be prepared by a cycloaddition reaction between alkene XXXVII and imine XXXIX. Alkene XXXVII can be prepared by the Wittig reaction of aldehyde XXXIV with phosphorane XXXV to form α,β-unsaturated ketone XXXVI. Enol ether XXXVII can be formed by trapping the enolate of XXXVI with TBSCl. The resulting TBS enol ether XL can be hydrolyzed with a mild acid to piperidinone XLI. Ketone XLI can be reduced to alcohol XLII. Those of skill in the art will recognize that ketone XLI and alcohol XLII can be further modified to yield compounds XLIII and XLIV, which represent a subset of structure I claimed herein.

Specific examples of the preparation of compounds according to formula I are described below.

### PREPARATION OF EXAMPLE 1

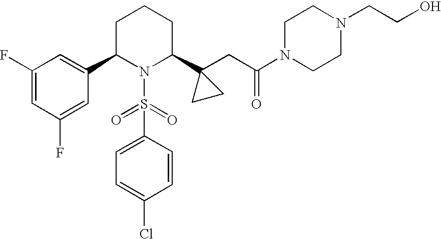

### Example 1

Methyl ester 1 was prepared in a manner similar to that of ethyl ester 5 of Example 173 in U.S. Ser. No. 10/358,898, as follows.
Preparation of Methyl Ester 1

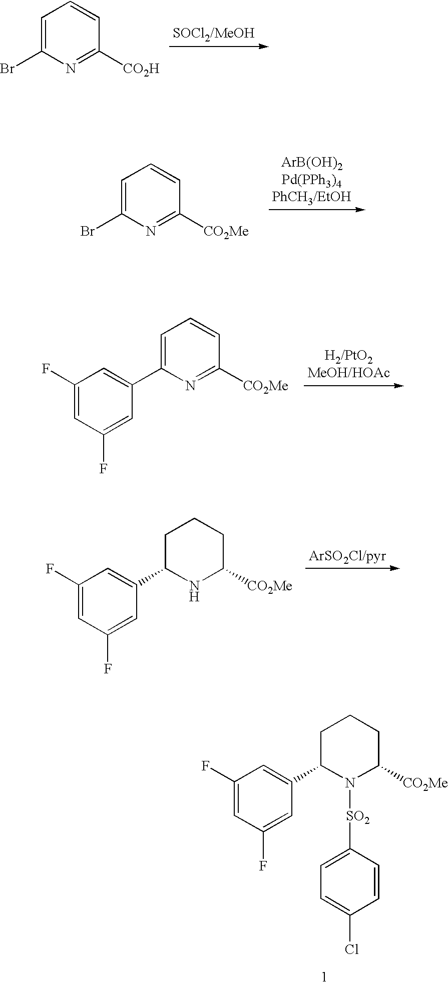

Step 1: To a cold (6° C.) mixture of 6-bromopicolinic acid (40.0 g, 198 mmol) in anhydrous methanol (750 mL), thionyl chloride (58 mL) was slowly added. The temperature was allowed to rise gradually to 34° C. while all of the 6-bromopicolinic acid dissolved. The mixture was refluxed for 5 hr. The solvent was removed under vacuum, and the residue was dissolved in 2 L of ethyl acetate and washed with 2 L of saturated sodium carbonate. The aqueous phase was re-extracted with 1.5 L of ethyl acetate. The combined organic phases were washed with 1.5 L of brine, dried over anhydrous MgSO₄, filtered and concentrated to dryness to give methyl 6-bromopicolinate (34.0 g) as an off-white solid.

Step 2: Methyl 6-bromopicolinate (43.8 g, 202.8 mmol) was heated in the presence of 3,5-difluorophenylboronic acid (40.6 g, 263.9 mmol), tetrakis(triphenylphosphine)palladium (23.5 g, 20.3 mmol) and sodium carbonate (45.2 g, 426 mmol) in toluene (572 mL) and ethanol (286 mL) at 80° C. for 16 hr. The mixture was cooled to room temperature and concentrated on a rotovap to remove solvents. The resulting residue was taken up in 1.3 L of DCM and washed twice with 800 mL of water. The combined aqueous phases were extracted with 500 mL of DCM. The organic phases were combined, then washed with brine, dried, and concentrated to provide approximately 90 g of a dark semi-solid material. The material was mixed with 280 mL of DCM and loaded onto a 1.5 L silica gel column (pre-packed using hexanes), and eluted with a gradient of 10-30% ethyl acetate in hexanes. After evaporation of the solvent and drying, 45.6 g of an off-white product was obtained.

Step 3: Under a hydrogen atmosphere, a solution of the product from Step 2 (45.6 g, 183.0 mmol) in methanol (2.4 L) and glacial acetic acid (600 mL) was stirred in the presence of platinum oxide (12.5 g) for 72 hr. The reaction mixture was then purged with nitrogen, and the reaction mixture was filtered and then concentrated under vacuum. The resulting residue was taken up water, treated with saturated sodium carbonate, and extracted with DCM. The organic phase was dried over anhydrous Na₂SO₄ and concentrated under vacuum to give a light yellow foam (44.5 g).

Step 4: A solution of the product of Step 3 (44.5 g, 174 mmol) in pyridine (300 mL) was treated with 4-chlorobenzenesulfonylchloride (110 g, 523 mmol). The mixture was heated at 60° C. for 4 hr, cooled to room temperature, concentrated under vacuum, and the resulting residue was subjected to flash-chromatography over silica gel (eluted with 10% ethyl acetate in hexanes) to provide 70.5 g of methyl ester 1 as a white powder.

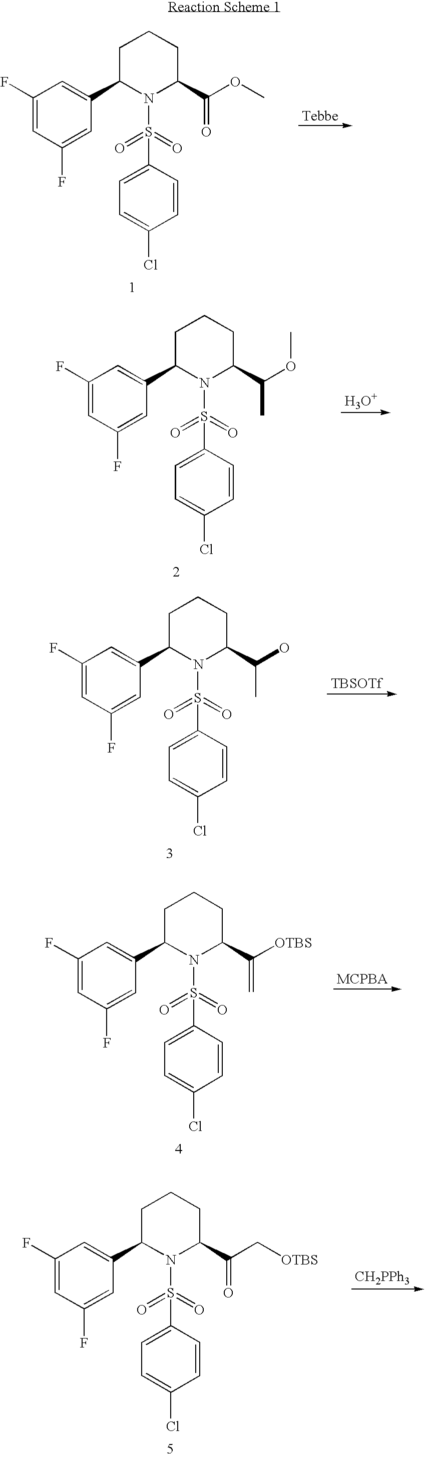

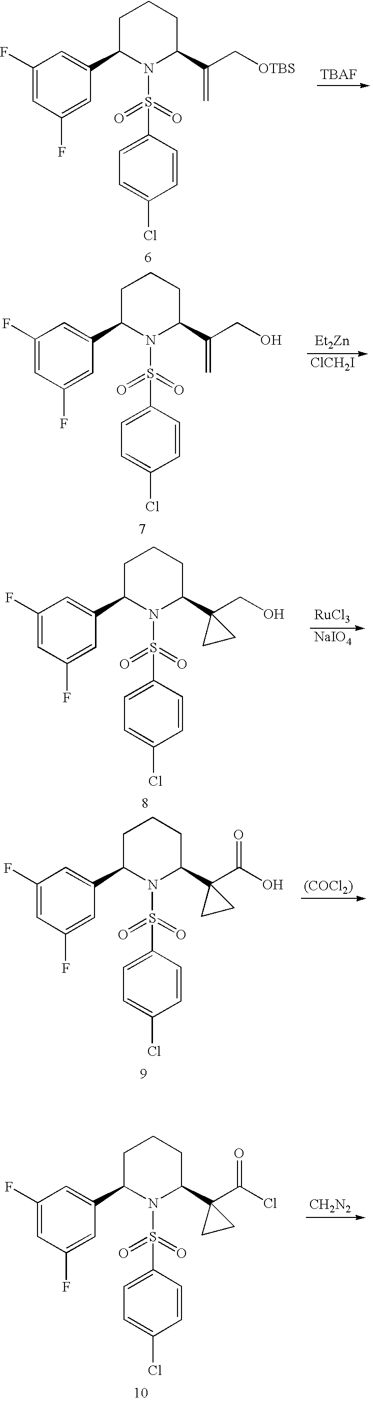

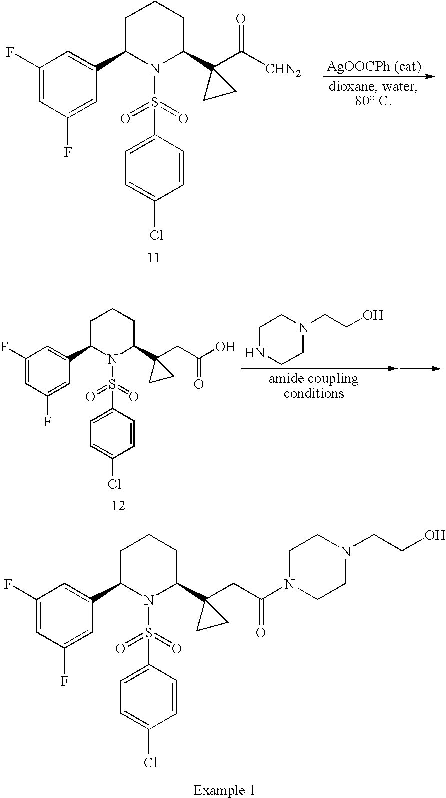

Step 1: To a solution of 7.0 g (16.3 mmol) of ester 1 in 20.0 mL of dry THF at 0° C., 50.0 mL of approximately 1M Tebbe reagent in toluene was added dropwise, followed by dropwise addition of 8.0 mL of pyridine. The mixture was stirred for 3 h at ambient temperature and quenched by cannulation of the mixture into approximately 200 g of crushed ice. Approximately 200 mL of DCM was then added, and the mixture was stirred for 30 min. The organic phase was then separated from the aqueous phase and the inorganic precipitate. The aqueous phase was re-extracted with DCM, and the organic phases combined. The combined organic phases were then dried over anhydrous sodium sulfate overnight, and then the solids were filtered out with Celite® (i.e., diatomaceous earth filter agent). The organic solvent was evaporated and the residue was subjected to flash chromatography (200 g of silica gel with 10-15% of ethyl acetate in hexanes as solvent) to provide approximately 6.0 g of enol ether 2.

Step 2: To a mixture of 1.0 g of enol ether 2 in 20.0 mL of acetone and 5 mL of DCM (added for solubility) was added 0.5 mL of TFA. The mixture was stirred for 45 min., over which time a precipitate fell out of solution. The volatile components of the mixture were removed, to provide a solid residue. The solid residue was re-dissolved in DCM and washed with 50% saturated aqueous NaHCO₃. The solution was then dried, concentrated, and passed through a 10 g silica gel plug using a mixture of 10% DCM, 10% EtOAc and 80% hexanes as the solvent, to provide 900 mg of ketone 3.

Step 3: To a mixture of 10.05 g (24.3 mmol) of ketone 3 in 140 mL of DCM was added 4.92 g (48.6 mmol) of triethylamine and 8.00 g (30.4 mmol) of tert-butyldimethylsilyltrifluromethanesulfonate. The mixture was stirred overnight, washed with ice-cold water, brine (saturated aqueous NaCl), dried over anhydrous sodium sulfate, concentrated, and then exposed to high vacuum at 60° C. over a period of 2 h to provide 13.9 g of crude TBS enol ether 4.

Step 4: To a solution of 13.9 g of crude TBS enol ether 4 in 100.0 mL of DCM was add, dropwise over 1 h, a solution of 4.54 g of MCPBA in 100.0 mL of DCM (technical MCPBA containing 57-86% of active material). The mixture was stirred for an additional 25 min. Because the reaction was incomplete by NMR analysis of a worked-up portion of the reaction mixture (using the work-up conditions described below), an additional 1.0 g of MCPBA in 10 mL of DCM was added, and the mixture was stirred for an additional 20 min. The mixture was then washed with saturated aqueous NaHCO₃, brine, dried over anhydrous sodium sulfate, and concentrated. The product was purified by chromatography over 120 g of silica gel using 10% of EtOAc in hexanes as solvent, to provide 9.3 g of ketone 5.

Step 5: To a suspension of 3.5 g (9.9 mmol) of methyltriphenylphosphonium bromide in THF (20 mL) at −40° C. was added 3.8 mL (9.6 mmol) of 2.5 M n-butyllithium in hexanes. The suspension was stirred for 5 min at −40° C., then stirred at 0° C. for 25 min. Then a solution of 2.0 g (3.7 mmol) of ketone 5 in dry THF (10.0 mL) was slowly added to the suspension. The resulting reaction mixture was stirred at 0° C. overnight. The reaction mixture was quenched with water, extracted with EtOAc, washed with water and brine and then dried over anhydrous magnesium sulfate. The concentrated product was purified by chromatography using a 0-15% gradient of ethyl acetate in hexanes to provide 950 mg of alkene 6.

Step 6: To a mixture of 1.0 g (2.0 mmol) of alkene 6 in THF (32.0 ml) was added 4.0 mL (4.0 mmol) of TBAF (1M in THF). The mixture was then stirred for 2 h. TLC analysis of the mixture (20% EtOAc/Hexane; silica stationary phase) showed that the reaction was complete, and that a more polar product was produced. The solvent was evaporated from the mixture, and the resulting residue was partitioned between DCM and water. The organic and aqueous phases were separated, and the organic phase was washed with water and brine, dried over anhydrous magnesium sulfate and concentrated to provide 1.0 g of crude alcohol 7.

Step 7: To a mixture of 20.0 mL of DCM and 14.0 mL (14 mmol) of 1M diethylzinc in hexane at 0° C. was added dropwise 1.0 mL (14 mmol) of chloroiodomethane. The mixture was stirred for 10 min at 0° C. and was then added dropwise a solution of 1.0 g of alcohol 7 in 20.0 mL of DCM. The mixture was stirred for 3.5 hours at ambient temperature. The reaction mixture was quenched with aqueous NH₄Cl (20%), extracted with DCM, and then washed with water and brine. The organic and aqueous phases were separated, and the organic phase was dried over anhydrous magnesium sulfate and concentrated. The product was purified by silica gel chromatography using a 0-25% gradient of ethyl acetate in hexanes to furnish 550 mg of cyclopropylmethanol 8.

Step 8: To 550 mg (1.24 mmol) of 8 in a mixture of 4.0 mL of CCl₄ and 4.0 mL of CH₃CN was added a solution of 1.1 g (4.98 mmol) of NaIO₄ in 6.0 mL of water, followed by the addition of 25 mg (0.12 mmol) of RuCl₃.H₂O. The resulting dark brown mixture was stirred overnight, then partitioned between DCM and water. The aqueous and organic phases were separated, and the aqueous phase was re-extracted with DCM. The organic phases were combined, then washed with brine, dried over anhydrous magnesium sulfate, and concentrated to furnish 560 mg of crude acid 9.

Step 9: To a solution of 560 mg (1.19 mmol) of acid 9 in DCM (18.0 ml) was added 0.625 mL (7.15 mmol) of oxalyl chloride. The mixture was stirred for 2.5 h. The solvent was removed and the resulting residue was placed under high vacuum for 5 h to provide 550 mg of acyl chloride 10.

Step 10: (a) Preparation of diazomethane. In a 250 mL flask, 14.0 mL of 5M NaOH and 67.0 mL of ether were added. The mixture was cooled to −5° C. (internal temperature) using an ice/NaCl bath. 3.0 g (20.4 mmol) of 1-methyl-3-nitro-1-nitrosoguanidine was added in portions, with shaking. The yellow ether layer was decanted into a pre-chilled flask and dried over several KOH pellets. The resulting diazomethane solution was kept in a loosely covered flask, cooled with ice/NaCl, and used within 10 min after generation.

(b) The diazomethane solution obtained in step (a) was added to a pre-cooled (0° C.) solution of 550 mg of acyl chloride 10 in 10.0 mL of THF. The mixture was left overnight at ambient temperature. 2.0 mL of acetic acid was then added to quench the remaining diazomethane. The reaction mixture was concentrated at room temperature under vacuum to a volume of approximately 15 mL, then diluted with 100 mL of DCM, washed with water, saturated aqueous NaHCO₃, dried over anhydrous sodium sulfate and concentrated under vacuum at a temperature of 30° C. The concentrated product was passed through a 5 g silica gel plug using 30% of ethyl acetate in hexanes to provide 300 mg of diazoketone 11.

Step 11: A mixture containing 250 mg of diazoketone 11, 8.0 mL of dioxane, 4.0 mL of water, and 15 mg of silver benzoate was heated at 75-80° C. for 2 h. The reaction mixture was then partitioned between DCM and water and the aqueous phase was re-extracted 5 times with DCM. The combined organic phases were dried over anhydrous sodium sulfate and concentrated. The concentrated product was then passed through a 5 g silica gel plug using a 0-5% gradient of methanol in DCM. Further purification was carried out by reverse-phase chromatography (C-4 phase, water-acetonitrile, 0.1% TFA) to provide 140 mg of acid 12.

Step 12: To a mixture of 15 mg (0.032 mmol) of acid 12 in 1.0 mL of DCM was added 5.2 mg of HOBT, 7.3 mg of EDCl followed by the addition of 5 mg of 2-piperazin-1-yl-ethanol and 7 pL of triethylamine. The mixture was stirred for 3 h and washed with water. The organic phase was then loaded on a preparative TLC plate (silica gel) using 5% MeOH in DCM as a solvent, and then re-purified by reverse-phase HPLC (C-4 column, acetonitrile-water) to provide 10 mg of Example 1.

Example 1: ¹H NMR (CDCl₃, 300 MHz) δ 7.82 (2H, d, J=8.05 Hz), 7.50 (2H, d, J=8.05 Hz), 7.12 (2H, d, J=7.12 Hz), 6.73 (1H, t, J=8.4 Hz), 5.10 (1H, s), 4.60 (1H, m), 3.77 (1H, m), 3.63 (2H, t, J=5.1 Hz), 3.51 (2H, m), 3.34 (1H, m), 3.18 (1H, d, J=16.8 Hz), 2.69-2.40 (6H, ser m), 1.99 (1H, m), 1.45-1.01 (7H, ser m), 0.70 (1H, m), 0.33 (1H, m), 0.14 (1H, m), −0.27 (1H, m). LCMS(ES) Retention time 3.73 min, m/z 582.1 (M+H)⁺.

### PREPATATION OF EXAMPLES 2-6

The following Examples 2-6 were prepared by reacting acid 12 with the appropriate cyclic amine (i.e., rather than with 2-piperazin-1-yl-ethanol) under conditions similar to those described in Step 12, above. Thus, for example, Example 2 was prepared by reacting acid 12 with piperidine rather than 2-piperazin-1-yl-ethanol.

| | | Retention time | Observed mass |
|---|---|---|---|
| Example | Structure | (min) | (m/z, M + H) |
| 2 | 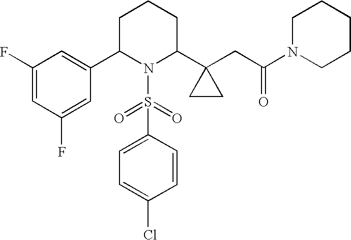 | 5.41 | 537.1 |
| 3 | 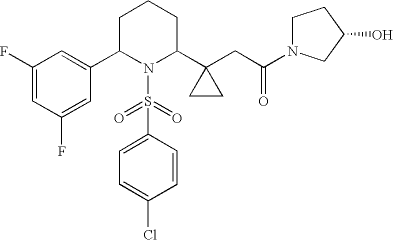 | 4.28 | 539.1 |
| 4 | 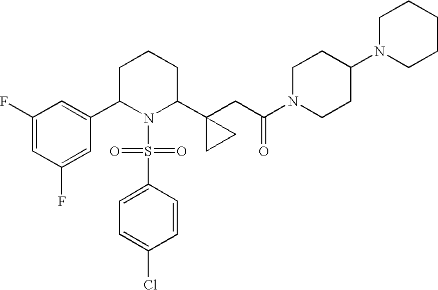 | 4.03 | 620.1 |
| 5 | 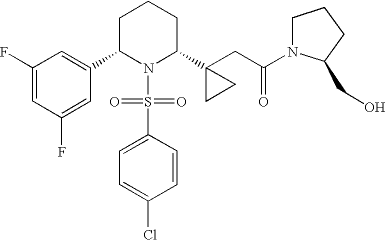 | 4.76 | 553.1 |
| 6 | 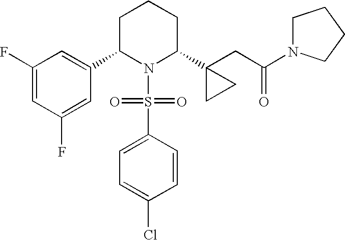 | 5.05 | 523.1 |

### PREPARATION OF EXAMPLES 7-18

TRhe following Examples 7-18 were prepared by reacting acid 9 or acyl chloride 10 prepared as described above, with the appropriate amine optionally in the presence of a base such as pyridine or triethylamine and also optionally in the presence of a catalyst such as dimethylaminopyridine (see for example Humphrey, J. M., Chamberlin, R., Chem. Rev., 1997, vol. 97, pp. 2243-2266).

| | | Retention time | Observed mass |
|---|---|---|---|
| Example | Structure | (min) | (m/z, M + H) |
| 7 | 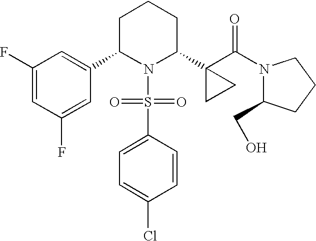 | 4.74 | 539.1 |
| 8 | 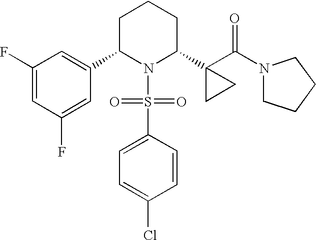 | 5.04 | 509.1 |
| 9 | 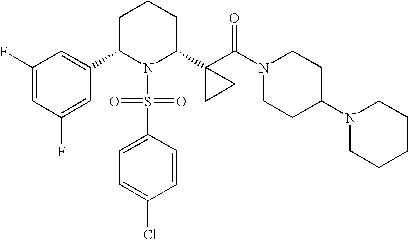 | 4.04 | 606.1 |
| 10 | 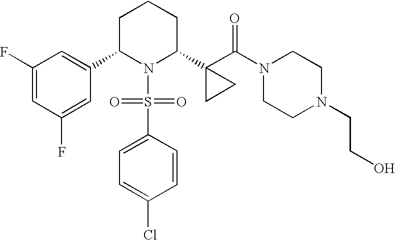 | 3.69 | 568.1 |
| 11 | 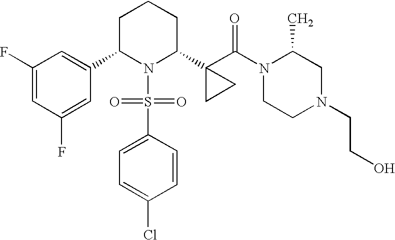 | 3.78 | 582.1 |
| 12 | 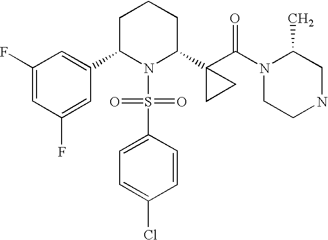 | 3.81 | 538.1 |
| 13 | 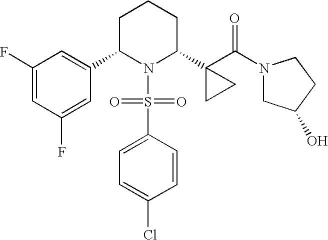 | 4.25 | 525.1 |
| 14 | 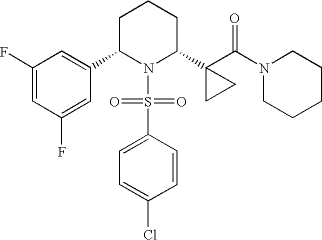 | 5.32 | 523.1 |
| 15 | 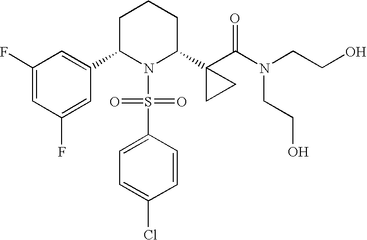 | 4.17 | 543.1 |
| 16 | 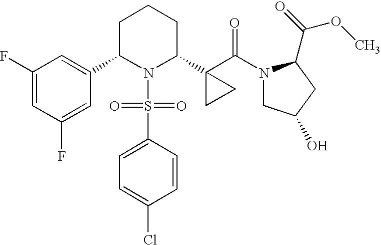 | 4.43 | 583.1 |
| 17 | 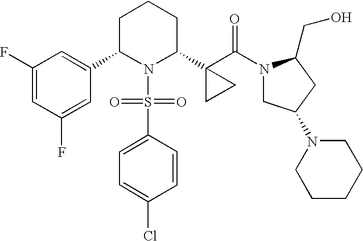 | 3.79 | 622.1 |
| 18 | 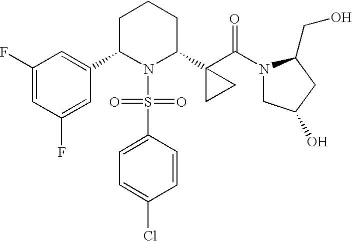 | 4.06 | 555.1 |

### PREPARATION OF EXAMPLE 19

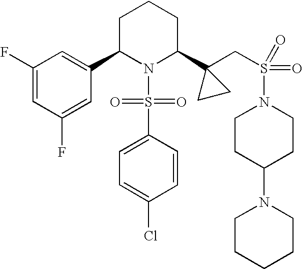

### Example 19

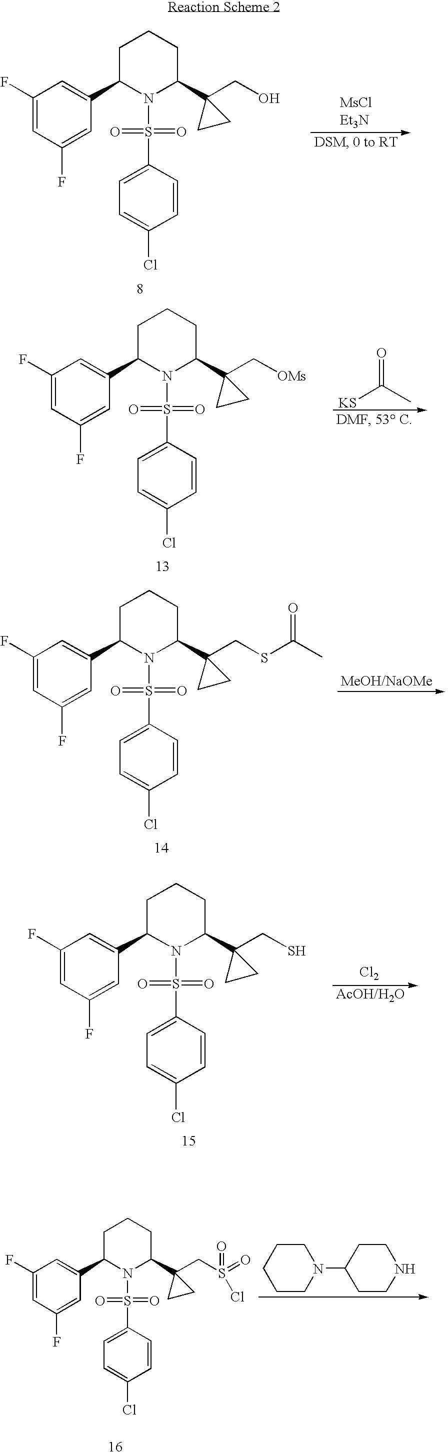

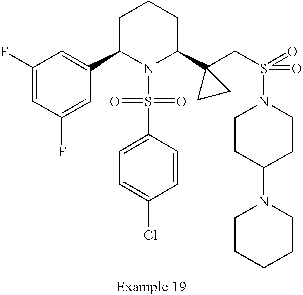

Step 1: To 690 mg (1.56 mmol) of compound 8 in DCM (15.0 mL) at 0° C., was added 0.434 mL (3.12 mmol) of triethylamine, followed by dropwise addition of 0.145 mL (1.87 mmol) of methanesulfonyl chloride. The mixture was stirred for 2 h, washed with aqueous NaHCO₃, and brine. The organic and aqueous phases were separated and the organic phase was then dried with anhydrous MgSO₄. The solvent was then evaporated to provide 850 mg of crude 13.

Step 2: A mixture of 850 mg (1.64 mmol) of compound 13 and 373 mg (3.27 mmol) of potassium thioacetate was stirred in 10.0 mL of DMF for 6 h at 55° C. The solvent was then evaporated, and the resulting residue was partitioned between DCM and water. The organic phase was washed with water and brine. Then, the solvent was evaporated and the resulting residue was purified by silica gel column chromatography using a 0-100% gradient of DCM in hexanes. 760 mg of thioacetate ester 14 was obtained.

Step 3: To a mixture of 760 mg (1.52 mmol) of thioacetate ester 14 in degassed MeOH (15 mL) and DCM (1 mL, added for solubility) was added 21 mg (0.38 mmol) of sodium methoxide. The mixture was heated up to 55° C. for 40 min under nitrogen, then the solvent was evaporated. The resulting residue was partitioned between DCM and water, and the aqueous phase was re-extracted three times with DCM and once with ethyl acetate. The organic phases were combined and then washed with saturated aqueous NH₄Cl and brine, and the solvent evaporated to form residue. 670 mg of crude thiol 15 was obtained and used without further purification in Step 4.

Step 4: Chlorine gas was bubbled into a solution of 90 mg of thiol 15 in 2 mL of AcOH/water (50/1 by volume) for 10 minutes. The solvent was then evaporated. The residue was partitioned between DCM and water, and then the organic and aqueous phases were separated. The organic phase was washed with aqueous NaHCO₃, dried, and the solvent was evaporated to provide crude sulfonyl chloride 16.

Step 5: The crude sulfonyl chloride 16 was dissolved in 1.5-2.0 mL of pyridine. This solution was treated with 92 mg of 4-piperidinopiperidine and then heated overnight at 60° C. The reaction mixture was then partitioned between aqueous saturated NaHCO₃ and DCM, and the organic phase was washed with water and brine, and dried. The organic and aqueous phases were then separated, and the solvent evaporated from the aqueous phase. The resulting residue was then purified by preparative TLC using 5% MeOH/DCM as the solvent, to provide 47 mg of Example 19.

Example 19: ¹H NMR (CDCl₃, 300 MHz) δ 7.89 (2H, d, J=8.8 Hz), 7.54 (2H, d, J=8.8 Hz), 7.04 (2H, d, J=7.3 Hz), 6.73 (1H, m), 5.18 (1H, s), 4.71 (1H, dd, J=2.9, 7.3 Hz), 3.99 (1H, d, J=13.2 Hz), 3.86 (2H, d, J=14.0 Hz), 2.83 (1H, dt, J=2.2, 12.0 Hz), 2.71 (1H, dt, J=2.2, 12.0 Hz), 2.54-2.35 (5H, ser m), 2.17 (1H, d, J=14.0 Hz), 2.20 (1H, m), 1.88 (1H, m), 1.72-1.55 (7H, ser m), 1.47-1.30 (5H, ser m), 1.14 (3H, m), 0.58 (1H, m), 0.28 (1H, m), 0.00(1H, m). LCMS(ES): Retention time 4.01 min; m/z=656.4 (M+H)⁺

### PREPARATION OF EXAMPLES 20-24

Examples 20-24 were prepared by methods similar to those used to prepare Example 19, except that the appropriate amine was used in place of 4-piperidinopiperidine in Step 5. Thus, for example, Example 21 was prepared with N-methylpiperazine instead of 4-piperidinopiperidine.

| | | Retention time | Observed mass |
|---|---|---|---|
| Example | Structure | (min) | (m/z, M + H) |
| 20 | 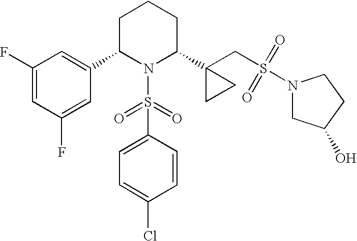 | 4.45 | 575.3 |
| 21 | 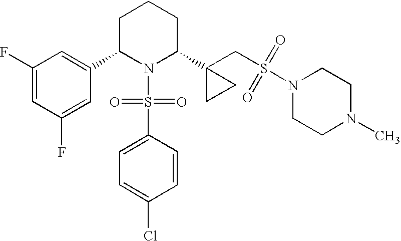 | 3.72 | 588.3 |
| 22 | 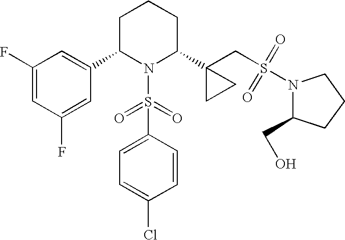 | 4.62 | 589.3 |
| 23 | 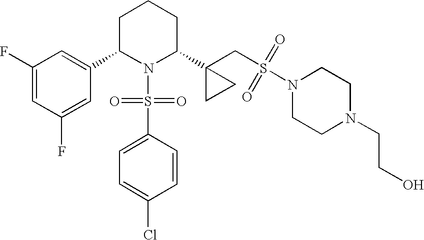 | 3.66 | 618.3 |
| 24 | 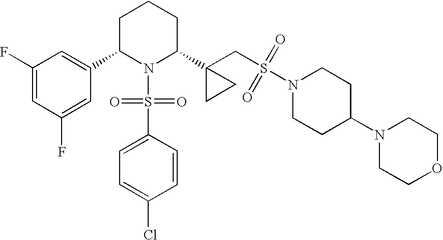 | 3.82 | 658.4 |

### PREPARATION OF EXAMPLE 25

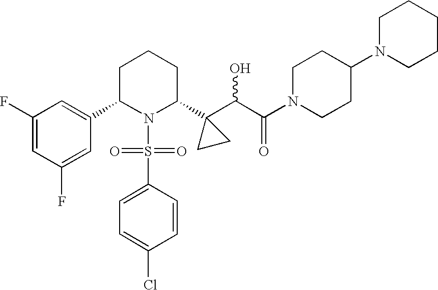

### Example 25

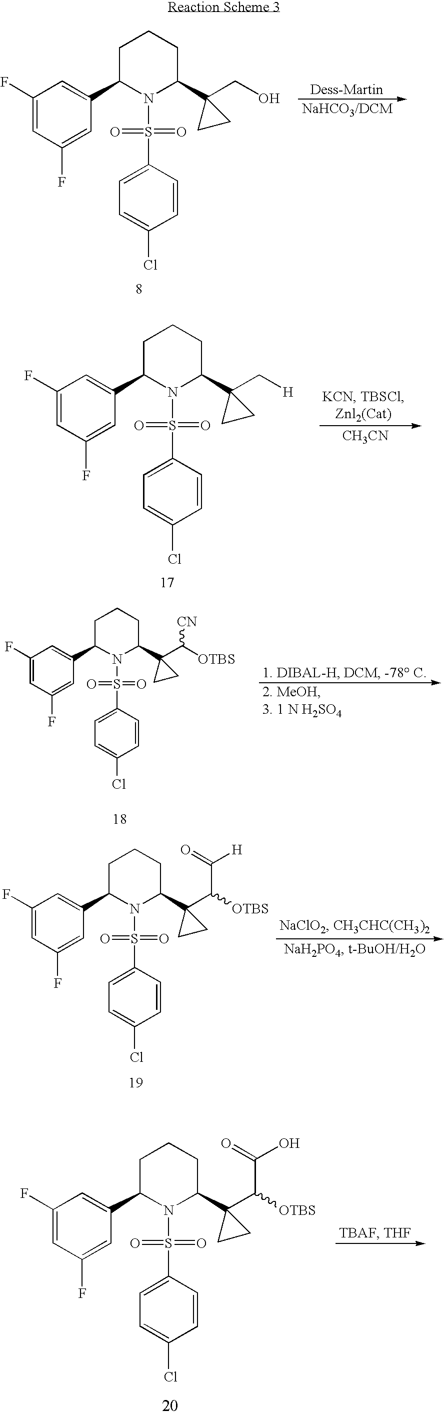

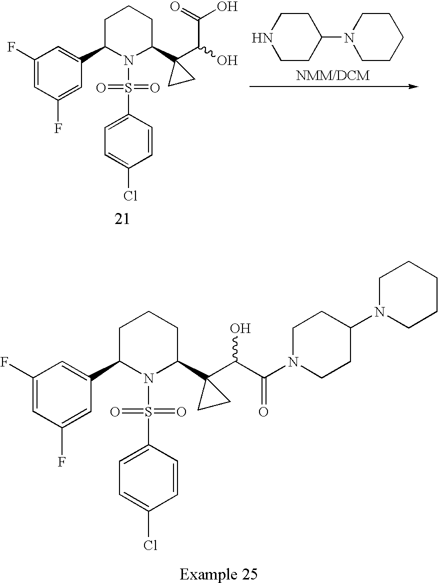

Step 1: To 110 mg (0.25 mmol) of alcohol 8 in 15 mL of DCM was added 127 mg (0.3 mmol) of Dess-Martin periodinane, followed by 31 mg (0.37 mmol) of NaHCO3. The reaction mixture was then stirred at RT for 2 hours and quenched with 0.4 g of sodium thiosulfate in sat. NaHCO3. The product was extracted with DCM, washed with water and brine, dried, concentrated, and purified by silica gel column chromatography using a 0-25% gradient of ethyl acetate in hexanes to furnish 92 mg of aldehyde 17.

Step 2: To 600 mg (1.37 mmol) of aldehyde 17 in 12 mL of acetonitrile was added 533 mg (8.2 mmol) of KCN, 22 mg (0.068 mmol) of Znl₂ and 269 mg (1.78 mmol) of TBDSCl. The reaction mixture was then stirred at 50° C. overnight.

The solvent was evaporated, and the resulting residue was re-dissolved in EtOAc and washed with water and brine to furnish compound 18.

Step 3: Compound 18 (638 mg, 1.1 mmol) was dissolved in 10 mL of DCM, chilled to −78° C. and treated with 1.78 mL (1.78 mmol) of DIBAL. The reaction mixture was allowed to warm up to 0° C. and was stirred at this temperature for 2 h. 1.5 mL of 1N H₂SO₄ was then added and the reaction mixture was stirred at 0° C. for another hour. The reaction mixture was washed with water and brine, dried, and concentrated to furnish aldehyde 19.

Step 4: To a mixture of 155 mg (0.265 mmol) of aldehyde 19 in 4 mL of tert-butanol and 1 mL of water at 0° C. was added 73 mg (0.532 mmol) of NaH₂PO₄, 0.118 mL of 2-methyl-2-butene and 77 mg (0.85 mmol) of sodium chlorite. The reaction mixture was stirred for 1.5 hours at RT. Saturated NH₄Cl (3 ml) and EtOAc (15 ml) were added. The organic layer was washed with brine, dried and concentrated to furnish carboxylic acid 20.

Step 5: 160 mg (0.267 mmol) of acid 20 was dissolved in 2 mL of THF and treated with 0.53 mL (0.534 mmol) of 1 M solution of TBAF in THF. After overnight stirring, reaction was quenched with water, extracted with EtOAc and DCM. The organic layer was washed with brine, dried and evaporated to furnish carboxylic acid 21.

Step 6: To a mixture of 65 mg (0.134 mmol) of carboxylic acid 21 and 34 mg (0.20 mmol) of 4-piperidinopiperidine in 2.0 mL of DCM at 0° C. was added 59 mg (0.134 mmol) [1,4′]-bipiperidine and 0.044 mL (0.402 mmol) of NMM. The mixture was stirred at RT for 5 hours, quenched with brine, extracted with EtOAc and DCM. The organic layer was washed with brine, dried and concentrated. The product was purified by preparative TLC using 6% of MeOH in DCM to furnish 33.5 mg of Example 25 as a diastereomeric mixture.

Example 25: (diastereomeric mixture) ¹H NMR (CDCl₃, 300 MHz) δ 7.90 (1.1 H, m), 7.82 (1.1 H, m), 7.54 (2.1 H, m), 7.14 (2.2 H, m), 7.04 (2.2 H, m), 6.72 (0.9 H, m), 5.04-4.80 (1.4 H, ser m), 4.72 (0.3 H, d), 4.63-4.44 (1.1 H, ser m), 4.36 (0.4 H, m), 4.26 (0.3 H, m), 4.10-3.77 (1.5 H, m), 3.59 (0.7 H, m), 3.52-3.32 (0.8 H, ser m), 3.00 (0.5 H, m), 2.85 (0.5 H, m), 2.69-2.34 (6.5 H, ser m), 2.1-0.7 (23.8 H, ser m), 0.65-0.22 (3.2 H, ser m), 0.12 (0.4 H, m), −0.38 (0.4 H, m), −0.50 (0.2 H, m). LCMS(ES) Single peak, retention time 3.63 min; m/z=636.2 (M+H)⁺.

### PREPARATION OF EXAMPLES 26-29

Examples 26-29 were prepared by methods similar to those used to prepare Example 25, except that the appropriate amine was used in place of 4-piperidinopiperidine in Step 6. Thus, for example, Example 26 was prepared with L-prolinol instead of 4-piperidinopiperidine.

| | | | Retention time | Observed mass |
|---|---|---|---|---|
| Example | | Structure | (min) | (m/z, M + H) |
| | 26 | 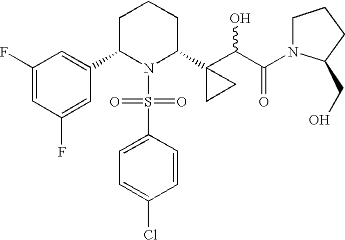 | 3.90 | 569.1 |
| | 27 | 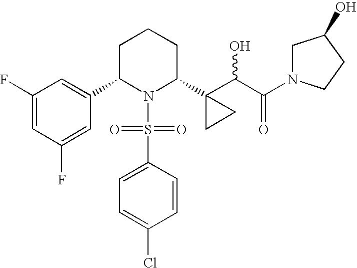 | 3.69 | 555.1 |
| | 28 | 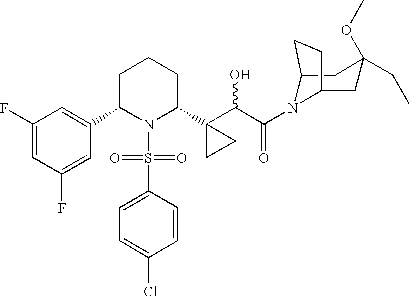 | 4.48 | 637.1 |
| | 29 | 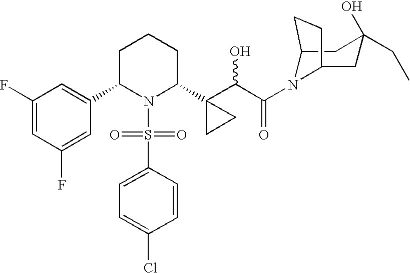 | 4.06 | 623.1 |

### PREPARATION OF EXAMPLES 30 and 31

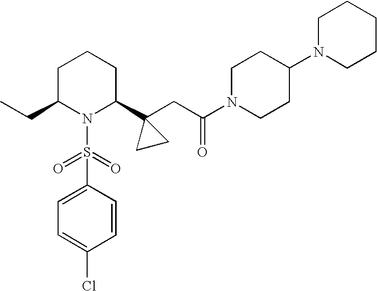

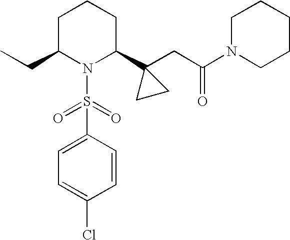

Methyl ester 22 was prepared in a manner similar to that of ethyl ester 5 of Example 173 in U.S. Ser. No. 10/358,898, as follows.
Preparation of methyl ester 25

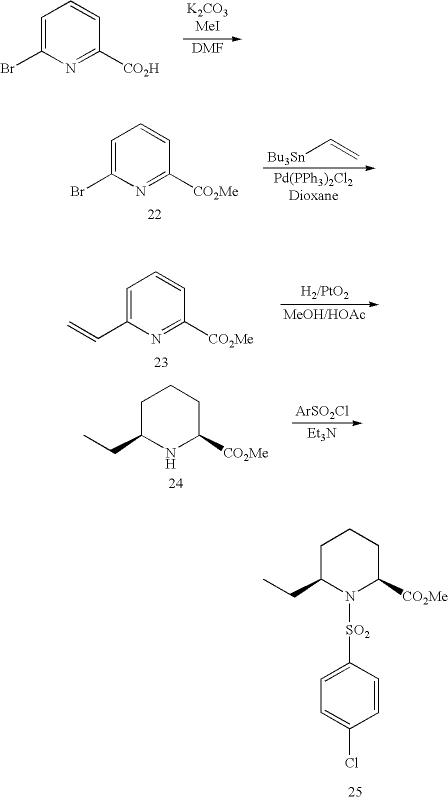

Step 1: A solution of 6-bromopicolinic acid (20.0 g, 99 mmol) in DMF (60 mL) was treated with K₂CO₃ (16.6 g, 120 mmol) followed by Mel (6.8 mL, 109 mmol). After 18 h, the reaction mixture was diluted with H₂O and extracted with EtOAc (2×). The combined organic extracts were washed with H₂O (3×), brine, dried over MgSO₄ and concentrated in vacuo to provide bromide 22 (16.9 g, 79%) as an off-white solid.

Step 2: A solution of bromide 22 (16.9 g, 78.2 mmol) in dioxane (120 mL) treated with tributyl(vinyl)tin (25.1 mL, 86 mmol) and Pd(Ph₃P)₂Cl₂ (2.0 g, 2.85 mmol) and heated to reflux. After 48 h, the reaction mixture was cooled to room temperature and concentrated in vacuo. The resulting residue was diluted with saturated aqueous NH₄Cl and extracted with EtOAc (3×). The combined organic extracts were stirred with a solution of KF (20 g) in H₂O (300 mL) for 30 min, filtered through Celite, and rinsed with EtOAc. The filtrate was washed with brine, dried over MgSO₄ and concentrated in vacuo. Flash chromatography (5→15% EtOAc/Hex) provided 23 (9.3 g, 73%) as a yellow solid.

Step 3: A solution of 23 (22.5 g, 138 mmol) in MeOH (400 mL) and glacial acetic acid (100 mL) was treated with platinum oxide (2.0 g) and stirred under H₂ (1 atm). After 36 h, the reaction mixture was filtered through Celite, rinsed with MeOH and concentrated in vacuo. The resulting residue was diluted with saturated sodium carbonate, and extracted with CH₂Cl₂ (2×). The combined organic extracts were washed with H₂O, dried over MgSO₄ and concentrated in vacuo to afford amine 24 (23.5 g, >99%) as a clear oil.

Step 4: A solution of amine 24 (23.5 g, 137 mmol) in DCE (400 mL) was treated with Et₃N (57 mL, 411 mmol), 4-chlorobenzenesulfonylchloride (34.8 g, 165 mmol) and heated to reflux. After 18 h, the reaction mixture was cooled to room temperature and washed sequentially with 1N HCl, 1N NaOH, H₂O, dried over MgSO₄ and concentrated in vacuo. Recrystallization from EtOAc/Hex (1:4) provided 25 (26.5 g). The filtrate was concentrated and recrystallized as above to provide a second batch (5.0 g), of which the filtrate was further recrystallized as above to provide a third batch (4.2 g, 75% total yield) of 25 as a white solid.

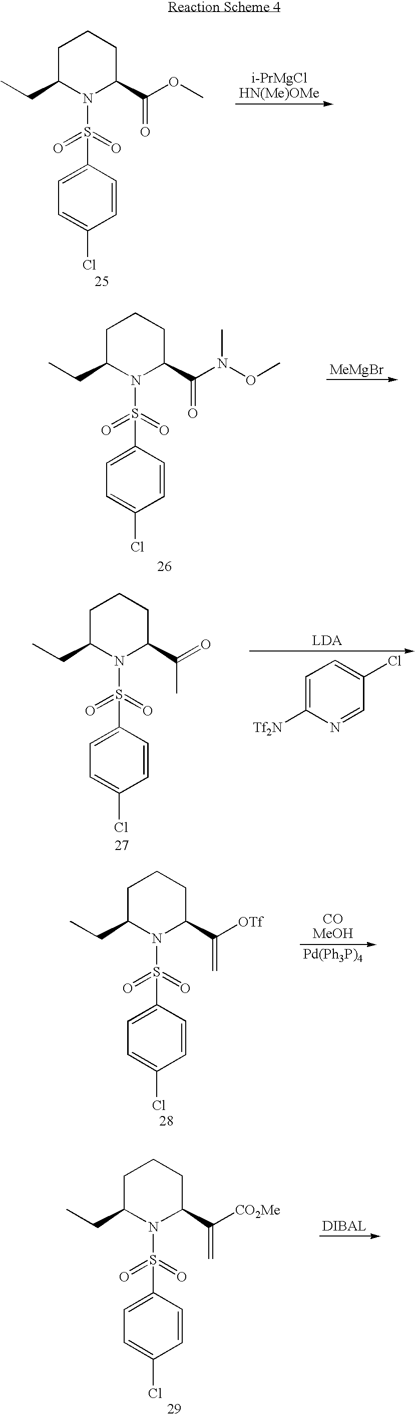

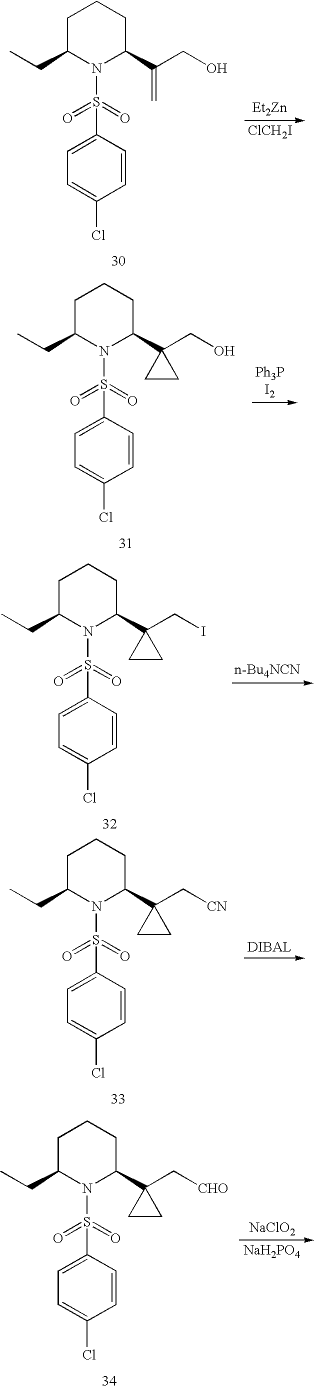

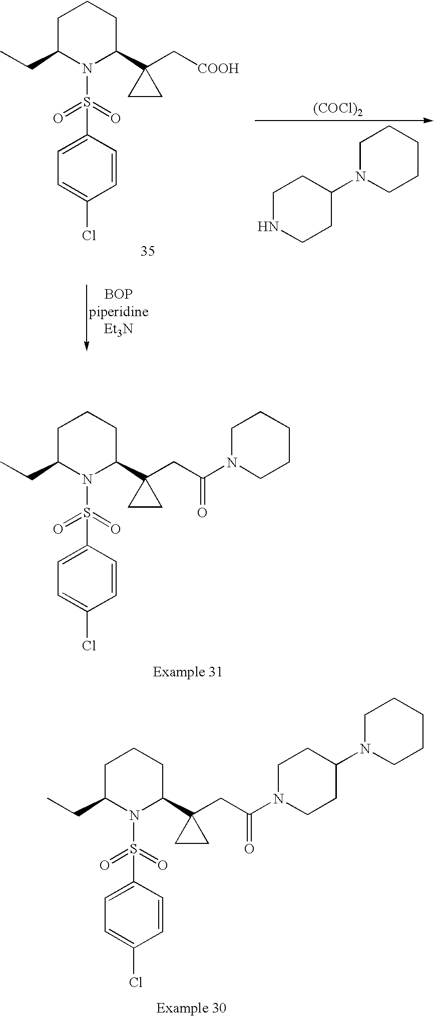

Step 1: A solution of ester 25 (10.0 g, 28.9 mmol) and N,O-dimethyhydroxylamine dimethylhydrochloride (4.24 g, 43.5 mmol) in THF (290 mL) at −20° C. treated dropwise with i-PrMgCl (43.5 mL, 87 mmol; 2.0 M in THF). The reaction mixture was warmed to ambient temperature over 2 h. After 2 additional h, the reaction mixture was quenched with saturated aqueous NH₄Cl and extracted with EtOAc (2×). The combined organic layers were washed with brine, dried over MgSO₄ and concentrated in vacuo to afford amide 26 (10.8 g, >99%) as a clear oil.

Step 2: A solution of crude amide 26 (10.8 g) in THF (260 mL) at 0° C. was treated with MeMgBr (19.3 mL, 58 mmol; 3.0 M in Et₂O). After 2 h, the reaction mixture was quenched with saturated aqueous NH₄Cl and extracted with Et₂O (2×). The combined organic layers were washed with brine, dried over MgSO₄ and concentrated in vacuo. Trituration (5% EtOAc/Hex) at 0° C. provided ketone 27 (5.99 g). The filtrate was concentrated and triturated as above to provide an additional 0.7 g (70% total yield) of ketone 27 as a white solid.

Step 3: A solution of ketone 27 (4.1 g, 12.43 mmol) in THF (80 mL) at −78° C. was treated with LDA (6.84 mL, 13.67 mmol; 2.0 M in heptane/THF/ethylbenzene). After 30 min, a solution of 2-[N,N-bis(trifluoromethylsulfonyl)amino]-5-chloropyridine (6.35 g, 16.16 mmol) in THF (20 mL) was added dropwise. After 4 h, the reaction mixture was warmed to 0° C. After 30 additional min, the reaction mixture was diluted with saturated aqueous NaHCO₃ and extracted with Et₂O (2×). The combined organic layers were washed with brine, dried over MgSO₄ and concentrated in vacuo to give crude 28. The solid residue was added to a solution of CH₃CN [purged for 45 min with CO(g)]. The solution was treated with n-Bu₃N (5.92 mL, 24.86 mmol), MeOH (60 mL), LiCl (0.53 g, 12.43 mmol) and (Ph₃P) ₄Pd (1.40 g, 1.25 mmol). The reaction mixture was evacuated and contacted with CO (1 atm), and heated to reflux under 1 atm CO. After 24 h, the reaction mixture was cooled to ambient temperature and concentrated to remove MeOH. The residue was diluted with Et₂O, 1N HCl and extracted with Et₂O (2×). The combined organic layers were washed with 1N HCl, saturated aqueous NaHCO₃, brine, dried over MgSO₄ and concentrated in vacuo. Flash chromatography (3→10% EtOAc/Hex) provided ester 29 (Rf=0.53, 10% EtOAc/Hex, 2.05 g, 44% over 2 steps) as a clear oil along with unreacted ketone 27 (Rf=0.63, 10% EtOAc/Hex, 930 mg).

Step 4: A solution of ester 29 (2.38 g, 6.40 mmol) in THF (60 mL) at −78° C. was treated with DIBAL (25 mL, 25 mmol; 1.0 M in Hex) and warmed to ambient temperature over 30 min. After an additional 2 h, the reaction mixture was quenched with 1N HCl and extracted with CH₂Cl₂ (3×). The combined organic layers were washed with H₂O, dried over MgSO₄ and concentrated in vacuo. Flash chromatography (20% EtOAc/Hex) afforded olefin 30 (2.03 g, 92%) as a clear oil.

Step 5: A solution of Et₂Zn (29 mL, 29 mmol; 1.0 M in Hex) in DCE (50 mL) at −20° C. was treated with chloroiodomethane (2.10 mL, 29 mmol) dropwise over 20 min. After an additional 5 min, a solution of olefin 30 (2.0 g, 5.90 mmol) in DCE (30 mL) was added dropwise and the reaction mixture warmed to ambient temperature over 30 min. After an additional 2.5 h, the reaction mixture was quenched with saturated aqueous NH₄Cl and extracted with CH₂Cl₂ (2×). The combined organic layers were washed with H₂O, dried over MgSO₄ and concentrated in vacuo to give alcohol 31 (1.98 g, 94%) as a white solid.

Step 6: A solution of alcohol 31 (650 mg, 1.82 mmol) in CH₃CN/Tol (30 mL, 1:2) at 0° C. was treated with Ph₃P (630 mg, 2.40 mmol), imidazole (375 mg, 5.5 mmol), and 12 (609 mg, 2.40 mmol). After 1.5 h, the reaction mixture was quenched with saturated aqueous NH₄Cl and extracted with Et₂O. The combined organic layers were washed with saturated aqueous NaHCO₃, brine, dried over MgSO₄ and concentrated in vacuo. Flash chromatography (5% EtOAc/Hex) afforded iodide 32 (600 mg, 70%) as a white solid.

Step 7: A solution of iodide 32 (2.73 g, 5.84 mmol) in CH₃CN (60 mL) was treated with n-Bu₄NCN (1.90 g, 7.0 mmol). After 1.5 h, the reaction mixture was diluted with H₂O and extracted with EtOAc (2×). The combined organic layers were washed with brine, dried over MgSO₄ and concentrated in vacuo. Flash chromatography (10% EtOAc/Hex) afforded nitrile 33 (1.85 g, 86%) as a white solid.

Step 8: A solution of nitrile 33 (1.38 g, 3.76 mmol) in CH₂Cl₂ (40 mL) at −78° C. was treated with DIBAL (5.6 mL, 5.6 mmol; 1.0 M in Hex) and warmed to −10° C. over 2 h. After an additional 1 h, the reaction mixture was quenched with 1N HCl, 2 mL MeOH and stirred vigorously. After 30 min, the biphasic solution was extracted with CH₂Cl₂ (3×). The combined organic layers were washed with H₂O, dried over MgSO₄ and concentrated in vacuo to provide crude 34 (1.4 g). The crude residue was dissolved in a solution of t-BuOH/H₂O (4:1, 40 mL), cooled to 0° C. and treated with NaH₂PO₄ (1.04 g, 7.52 mmol), 2-methyl-2-butene (9.4 mL, 18.8 mmol; 2.0 M in THF), NaClO₂ (1.09 g, 12.0 mmol) and warmed to ambient temperature. After 45 min, the reaction mixture was diluted with saturated aqueous NH₄Cl and extracted with EtOAc (3×). The combined organic layers were washed with H₂O, brine, dried over MgSO₄ and concentrated in vacuo to afford acid 35 (1.56 g, >99%) as a white solid.

Step 9: A solution of acid 35 (30 mg, 0.078 mmol) in CH₂Cl₂ (1 mL) was treated with oxalyl chloride (60 μL, 0.70 mmol). After 30 min, the reaction mixture was concentrated in vacuo, diluted with CH₂Cl₂ (1 mL) and treated with Et₃N (98 μL, 0.70 mmol) followed by 4-piperidinopiperidine (27 mg, 0.16 mmol). After 3 h, the reaction mixture was directly purified via preparative TLC (5% MeOH/CH₂Cl₂) to provide Example 30 (25 mg, 60%) as a yellow oil.

Example 30: ¹H NMR (CDCl₃, 400 MHz) δ 7.77 (dd, J=8.1, 4.4 Hz, 2 H), 7.46 (dd, J=8.8, 5.9 Hz, 2 H), 4.75-4.54 (m, 2 H), 3.92 (m, 1 H), 3.73 (m, 1 H), 3.40 (d, J=16.2 Hz, 1 H), 2.99 (m, 1 H), 2.59-2.43 (m, 7 H), 1.94-1.67 (m, 6 H), 1.58-1.43 (m, 5 H), 1.22-1.02 (m, 6 H), 0.97 (t, J=7.3 Hz, 3 H), 0.90-0.50 (m, 5 H). LCMS(ES) retention time 3.62 min, m/z 536.1 (M+H⁺).

Step 9a: A solution of acid 35 (40 mg, 0.104 mmol) in CH₂Cl₂ (2 mL) was treated with piperidine (15 μL, 0.156 mmol), Et₃N (31 μL, 0.22 mmol) and BOP reagent (60 mg, 0.135 mmol). After 18 h, the reaction mixture was directly purified via preparative TLC (25% EtOAc/Hex) to provide Example 31.

Example 31: (35.3 mg, 75%) as a yellow solid. LCMS(ES) retention time 4.38 min, m/z 453.1 (M+H⁺).

### PREPARATION OF EXAMPLES 32-33

The following Examples 32-33 were prepared by reacting acid 35 with the appropriate cyclic amine (i.e., rather than with 2-piperidinopiperidine) under conditions similar to those described in Step 9, above. Thus, for example, Example 33 was prepared by reacting acid 35 with (+/−)-1,4-diazabicyclo[4.4.0]decane rather than 2-piperidinopiperidine.

| | | Retention time | Observed mass |
|---|---|---|---|
| Example | Structure | (min) | (m/z, M + H) |
| 32 | 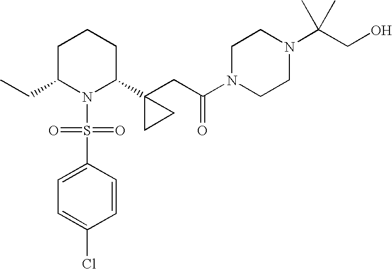 | 3.38 | 526.1 |
| 33 | 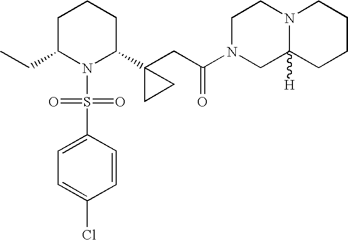 | 3.56 | 508.1 |

### PREPARATION OF EXAMPLES 34-38

The following Examples 34-38 were prepared by reacting acid 35 with the appropriate cyclic amine (i.e., rather than with piperidine) under conditions similar to those described in Step 9a, above. Thus, for example, Example 34 was prepared by reacting acid 35 with (R)-(+)-3-pyrrolidinol rather than piperidine.

| | | Retention time | Observed mass |
|---|---|---|---|
| Example | Structure | (min) | (m/z, M + H) |
| 34 | 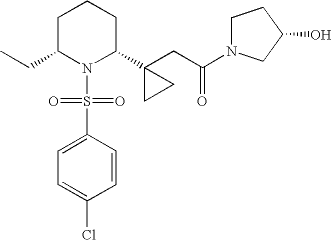 | 3.75 | 455.1 |
| 35 | 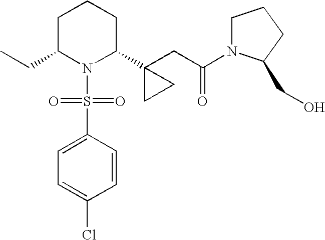 | 4.05 | 469.1 |
| 36 | 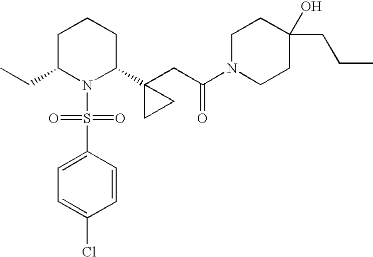 | 4.56 | 511.13 |
| 37 | 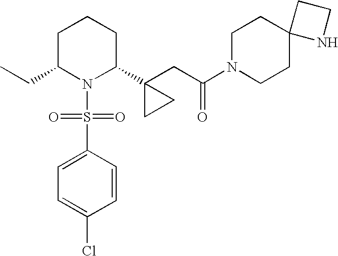 | 3.45 | 494.1 |
| 38 | 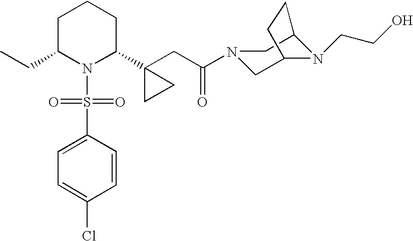 | 3.42 | 524.1 |

### PREPARATION OF EXAMPLES 39-40

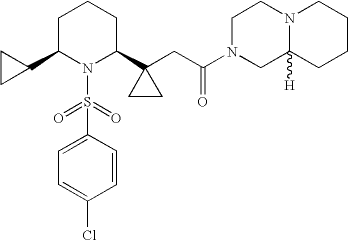

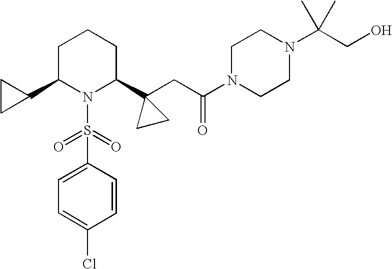

Olefin 36 was prepared by the method given for Example 1 in U.S. Pat. No. 0,229,902.

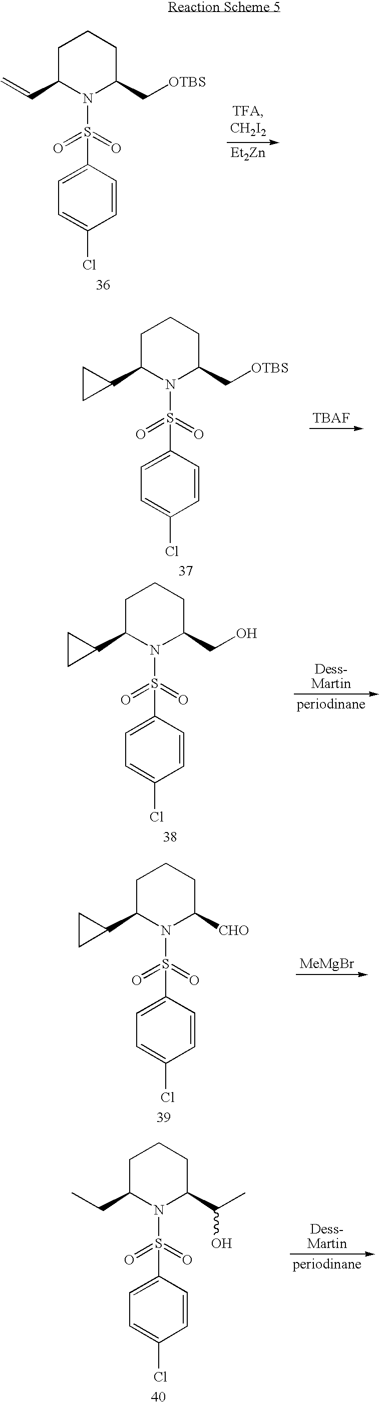

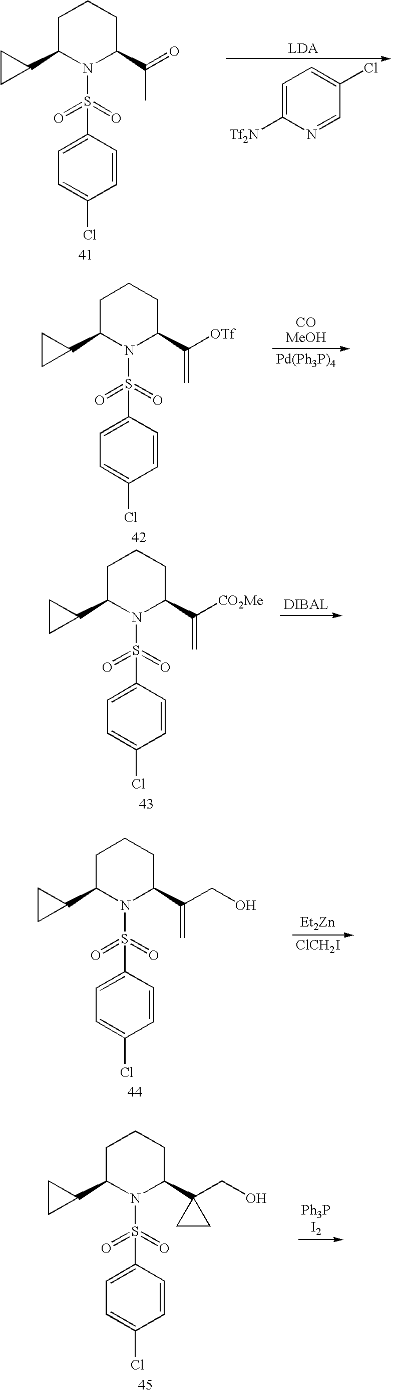

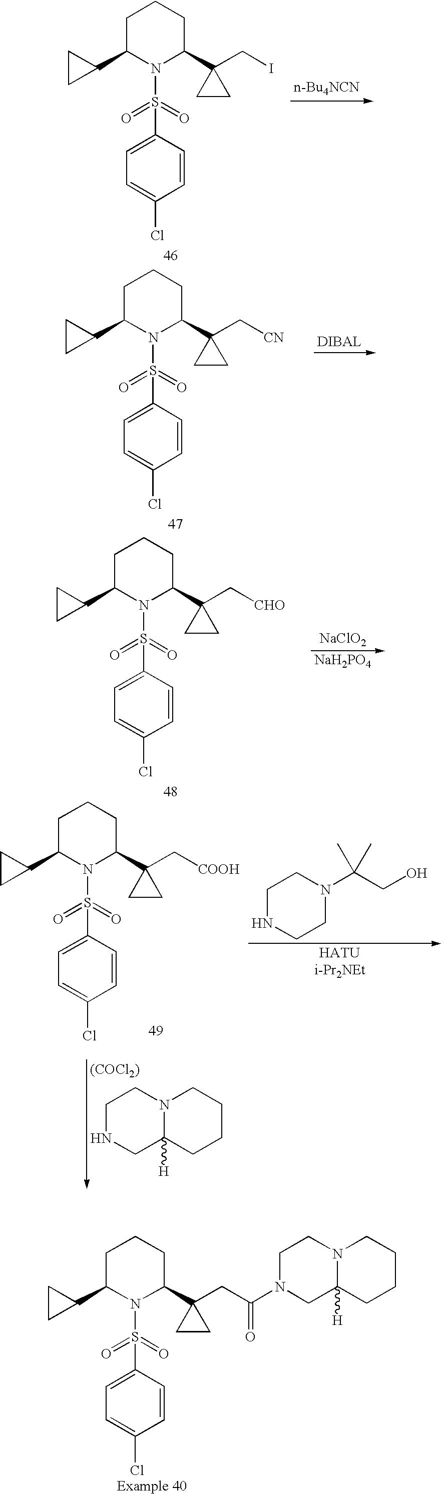

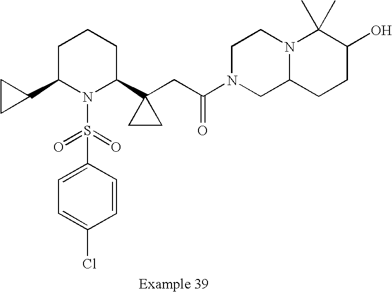

Step 1: A solution of Et₂Zn (48.4 mL, 48.4 mmol; 1.0 M in Hex) in CH₂Cl₂ (20 mL) at 0° C. was treated with TFA (3.7 mL, 48.4 mmol). After 5 min, CH₂I₂ (3.9 mL, 48.4 mmol) was added. After an additional 5 min, a solution of olefin 36 (5.2 g, 12.1 mmol) in CH₂Cl₂ (40 mL) was added and the reaction mixture was warmed slowly to ambient temperature. After 2 h, the reaction mixture was quenched with MeOH, diluted with H₂O and extracted with CH₂Cl₂ (4×) followed by EtOAc (2×). The combined organic layers were washed with dried over MgSO₄ and concentrated in vacuo to afford silyl ether 37 (6.1 g, >99%) as a clear oil.

Step 2: A solution of silyl ether 37 (25 g, 56.3 mmol) in THF (250 mL) at 0° C. was treated with TBAF (110 mL, 110 mmol; 1.0 M in THF) and warmed to ambient temperature. After 18 h, the reaction mixture was concentrated in vacuo and diluted with 1N HCl and Et₂O, and extracted with Et₂O (3×). The combined organic layers were washed with 1N HCl (2×), H₂O, brine, dried over MgSO₄ and concentrated in vacuo. Flash chromatography (0→5% MeOH/CH₂Cl₂) provided crude alcohol 38 (26.2 g) as a white solid.

Step 3: A solution of crude alcohol 38 (26.2 g) in CH₂Cl₂ (500 mL) at 0° C. was treated with pyridine (8.7 mL, 101 mmol) followed by Dess-Martin periodinane (34 g, 80 mmol) and warmed to ambient temperature. After 2.5 h, H₂O (3 drops) was added. After an additional 30 min, the reaction mixture was concentrated in vacuo, diluted with Et₂O, and washed with saturated aqueous NaHCO₃/Na₂S₂O₃ (1:1). The aqueous layer was back-extracted with Et₂O (2×). The combined organic layers were washed with 1N HCl (2×), saturated aqueous NaHCO₃, brine, dried over MgSO₄ and concentrated in-vacuo. Trituration (2:3:25 EtOAc/Et₂O/Hex) at 0° C. provided crude aldehyde 39 (20.3 g) as a white solid.

Step 4: A solution of aldehyde 39 (20.3 g) in THF (500 mL) at 0° C. was treated with MeMgBr (28 mL, 84 mmol; 3.0 M in Et₂O) and warmed to ambient temperature over 1 h. After an additional 15 min, the reaction mixture was quenched with saturated aqueous NH₄Cl and concentrated in vacuo. The aqueous solution was extracted with Et₂O (2×). The combined organic layers were washed with saturated aqueous NaHCO₃, brine, dried over MgSO₄ and concentrated in vacuo to give alcohol 40 (17.8 g, 92% over 3 steps) as a white solid.

Step 5: A solution of alcohol 40 (17.8 g, 51.8 mmol) in CH₂Cl₂ (500 mL) at 0° C. was treated with pyridine (6.6 mL, 77 mmol) followed by Dess-Martin periodinane (28.8 g, 68 mmol) and warmed to ambient temperature. After 4 h, the reaction mixture was concentrated in vacuo, diluted with Et₂O, and washed with saturated aqueous NaHCO₃/Na₂S₂O₃ (1:1). The aqueous layer was back-extracted with Et₂O (2×). The combined organic layers were washed with 1N HCl (2×), saturated aqueous NaHCO₃, brine, dried over MgSO₄ and concentrated in vacuo. Trituration (10% EtOAc/Hex) at 0° C. provided ketone 41 (13.5 g). The filtrate was concentrated and triturated as above to provide a second crop (2.1 g, total yield 88%) of 41 as a white solid.

Step 6: A solution of ketone 41 (2.56 g, 7.50 mmol) in THF (50 mL) at −78° C. was treated with LDA (4.1 mL, 8.25 mmol; 2.0 M in heptane/THF/ethylbenzene). After 30 min, a solution of 2-[N,N-bis(trifluoromethylsulfonyl)amino]-5-chloropyridine (3.8 g, 9.68 mmol) in THF (10 mL) was added dropwise. After 4 h, the reaction mixture was warmed to 0° C. After an additional 2.5 h, the reaction mixture was diluted with saturated aqueous NaHCO₃ and extracted with Et₂O (2×). The combined organic layers were washed with brine, dried over MgSO₄ and concentrated in vacuo to give crude 42. The solid residue was added to a solution of CH₃CN [purged for 30 min with CO(g)]. The solution was treated with n-Bu₃N (3.57 mL, 15 mmol), MeOH (25 mL), LiCl (0.32 g, 7.5 mmol) and Ph₃P (0.39 g, 1.5 mmol) and Pd(dba)₂ (0.43 g, 0.75 mmol). The reaction mixture was evacuated and contacted with CO (1 atm), and heated to reflux under 1 atm CO. After 12 h, the reaction mixture was cooled to ambient temperature and concentrated to remove MeOH. The residue was diluted with Et₂O, 1N HCl and extracted with Et₂O (3×). The combined organic layers were washed with 1N HCl, saturated aqueous NaHCO₃, brine, dried over MgSO₄ and concentrated in vacuo. Flash chromatography (3→10% EtOAc/Hex) provided ester 43 (Rf=0.53, 10% EtOAc/Hex, 790 mg, 27% over 2 steps) as a clear oil along with unreacted ketone 41 (Rf=0.63, 10% EtOAc/Hex, 730 mg).

Step 7: A solution of ester 43 (1.79 g, 4.66 mmol) in THF (50 mL) at −78° C. was treated with DIBAL (14 mL, 14 mmol; 1.0 M in Hex) and warmed to ambient temperature over 30 min. After an additional 1 h, the reaction mixture was cooled to 0° C., quenched with 1N HCl and extracted with CH₂Cl₂ (3×). The combined organic layers were washed with H₂O, dried over MgSO₄ and concentrated in vacuo. Flash chromatography (20% EtOAc/Hex) afforded olefin 44 (1.5 g, 90%) as a clear oil.

Step 8: A solution of Et₂Zn (17 mL, 17 mmol; 1.0 M in Hex) in DCE (30 mL) at −20° C. was treated with chloroiodomethane (1.24 mL, 17 mmol) dropwise over 20 min. After an additional 5 min, a solution of olefin 44 (1.5 g, 4.21 mmol) in DCE (20 mL) was added dropwise and the reaction mixture warmed to ambient temperature over 30 min. After an additional 2.5 h, the reaction mixture was quenched with saturated aqueous NH₄Cl and extracted with CH₂Cl₂ (2×). The combined organic layers were washed with H₂O, dried over MgSO₄ and concentrated in vacuo to give alcohol 45 (1.57 g, >99%) as a clear oil.

Step 9: A solution of alcohol 45 (1.5 g, 4.21 mmol) in CH₃CN/Tol (40 mL, 1:2) at 0° C. was treated with Ph₃P (1.3 g, 5.0 mmol), imidazole (0.82 g, 12.0 mmol), and 12 (1.27 g, 5.0 mmol). After 20 min, the reaction mixture was quenched with saturated aqueous NH₄Cl and extracted with Et₂O (2×). The combined organic layers were washed with saturated aqueous NaHCO₃, brine, dried over MgSO₄ and concentrated in vacuo. Flash chromatography (3% EtOAc/Hex) afforded iodide 46 (1.6 g, 79%) as a clear oil.

Step 10: A solution of iodide 46 (1.6 g, 3.33 mmol) in CH₃CN (40 mL) was treated with n-Bu₄NCN (1.4 g, 5.1 mmol). After 2 h, the reaction mixture was diluted with saturated aqueous NH₄Cl and extracted with Et₂O (3×). The combined organic layers were washed with H₂O, brine, dried over MgSO₄ and concentrated in vacuo. Flash chromatography (10% EtOAc/Hex) afforded nitrile 47 (1.0 g, 79%) as a white solid.

Step 11: A solution of nitrile 47 (1.0 g, 2.64 mmol) in CH₂Cl₂ (30 mL) at −78° C. was treated with DIBAL (4.7 mL, 4.7 mmol; 1.0 M in Hex) and warmed to 0° C. over 1 h. After an additional 15 min, the reaction mixture was quenched with 1N HCl, 2 mL MeOH and stirred vigorously. After 30 min, the biphasic solution was extracted with CH₂Cl₂ (3×). The combined organic layers were washed with H₂O, dried over MgSO₄ and concentrated in vacuo to provide crude 48 (950 mg). The crude residue was dissolved in a solution of t-BuOH/H₂O (4:1, 30 mL), cooled to 0° C. and treated with NaH₂PO₄ (730 mg, 5.28 mmol), 2-methyl-2-butene (6.6 mL, 13.2 mmol; 2.0 M in THF), NaClO₂ (764 mg, 8.45 mmol) and warmed to ambient temperature. After 1.5 h, the reaction mixture was diluted with saturated aqueous NH₄Cl and extracted with EtOAc (3×). The combined organic layers were washed with H₂O, dried over MgSO₄ and concentrated in vacuo to afford acid 49 (1.03 g, 98%) as a white solid.

Step 12: A solution of acid 49 (50 mg, 0.125 mmol) in CH₂Cl₂ (2 mL) at 0° C. was treated i-Pr₂NEt (110 μL, 0.625 mmol) and HATU (61 mg, 0.163 mmol). After 10 min, the dihydrochloride salt of 2-methyl-2-piperazin-1-yl-propan-1-ol (43 mg, 0.188 mmol, WO 2001007441) was added. After 18 h, the reaction mixture was diluted with saturated aqueous NH₄Cl and extracted with CH₂Cl₂ (2×). The combined organic layers were washed with saturated aqueous NaHCO₃, dried over MgSO₄ and concentrated in vacuo. Preparative TLC (0.5:4.5:95 NH₄OH/MeOH/CH₂Cl₂) afforded Example 39 as a yellow solid, which was dissolved in Et₂O (2 mL) and treated with HCl (1.0 mL, 1N in Et₂O) followed by trituration to provide hydrochloride salt (23.4 mg, 32%) as a yellow solid.

Example 39: ¹H NMR (free base) (CDCl₃, 400 MHz) δ 7.71 (d, J=8.8 Hz, 2 H), 7.44 (d, J=8.8 Hz, 2 H), 4.54 (d, J=6.6 Hz, 1 H), 3.67-3.55 (m, 4 H), 3.42-3.34 (m, 2 H), 2.97 (m, 1 H), 2.82-2.52 (m, 6 H), 1.95 (m, 1 H), 1.66 (m, 1 H), 1.56-1.43 (m, 2H), 1.21-0.90 (m, 7 H), 0.86 (m, 1 H), 0.75-0.52 (m, 6 H), 0.23 (m,1 H). LCMS(ES): Retention time 3.31 min, m/z 538.3 (M+H⁺).

Step 12a: A solution of acid 49 (50 mg, 0.125 mmol) in CH₂Cl₂ (2 mL) was treated with oxalyl chloride (100 μL, 1.16 mmol). After 20 min, the reaction mixture was concentrated in vacuo, diluted with CH₂Cl₂ (1 mL) and treated with Et₃N (130 μL, 1.20 mmol) followed by (+/−)-1,4-diazabicyclo[4.4.0]decane (140 mg, 1.0 mmol). After 18 h, the reaction mixture was diluted with saturated aqueous NH₄Cl and extracted with CH₂Cl₂ (2×). The combined organic layers were washed with saturated aqueous NaHCO₃, dried over MgSO₄ and concentrated in vacuo. Preparative TLC (0.5:4.5:95 NH₄OH/MeOH/CH₂Cl₂), afforded Example 40 (42.0 mg, 65%) as a yellow oil.

Example 40: LCMS(ES): retention time 3.45 min, m/z 520.3 (M+H⁺).

### PREPARATION OF EXAMPLES 41-42

The following Examples 40-41 were prepared by reacting acid 49 with the appropriate cyclic amine (i.e., rather than with 2-methyl-2-piperazin-1-yl-propan-1-ol) under conditions similar to those described in Step 12, above. Thus, for example, Example 41 was prepared by reacting acid 49 with 2-(2S)-2-Methyl-piperazin-1-yl)-ethanol rather than 2-methyl-2-piperazin-1-yl-propan-1-ol.

| | | Retention | Observed mass |
|---|---|---|---|
| Example | Structure | time (min) | (m/z, M + H) |
| 41 | 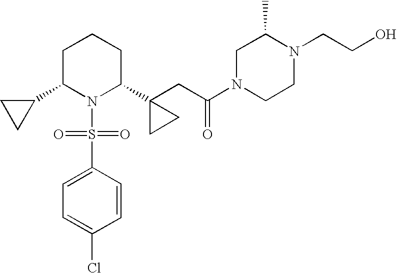 | 3.25 | 536.3 |
| 42 | 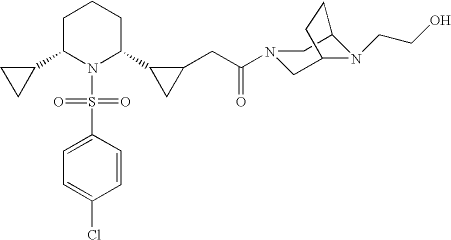 | 3.25 | 536.3 |

### PREPARATION OF EXAMPLES 43-44

The following Examples 43-44 were prepared by reacting acid 49 with the appropriate cyclic amine (i.e., rather than with (+/−)-1,4-diazabicyclo[4.4.0]decane) under conditions similar to those described in Step 12a, above. Thus, for example, Example 44 was prepared by reacting acid 49 with octahydro-pyrrolo[1,2-a]pyrazine rather than (+/−)-1,4-diazabicyclo[4.4.0]decane.

| | | | Observed |
|---|---|---|---|
| | | Retention | mass (m/z, |
| Example | Structure time (min) | M + H) | |
| 43 | 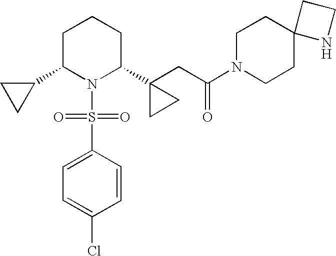 | 3.50 | 506.1 |
| 44 | 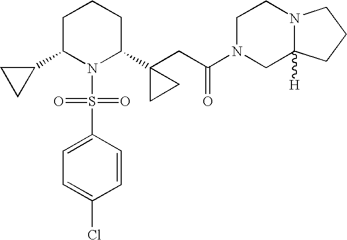 | 3.37 | 506.3 |

### PREPARATION OF EXAMPLE 45

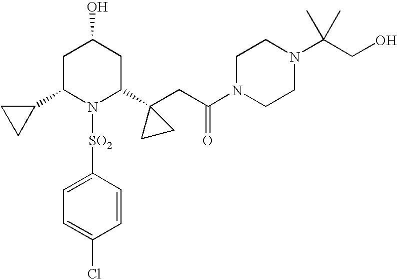

### Example 45

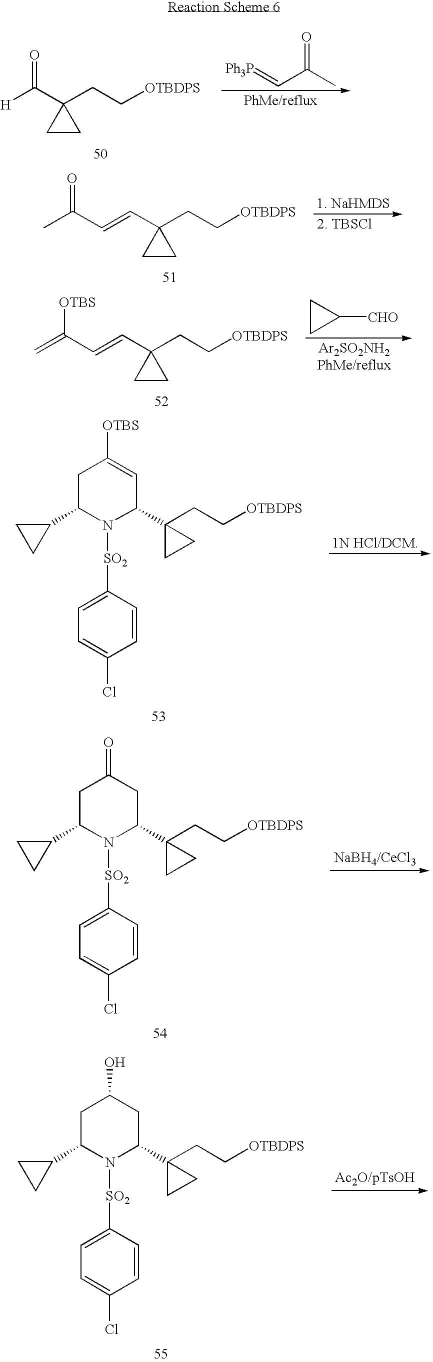

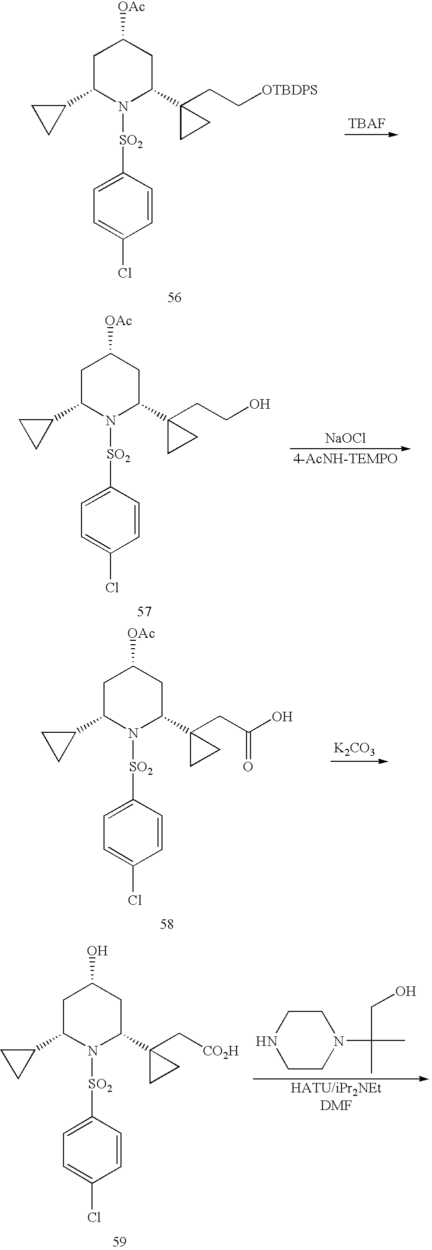

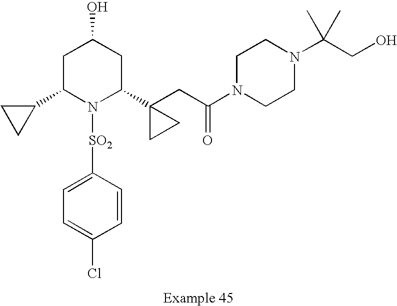

Step 1: Cyclopropanecarboxaldehyde 50 was obtained as described in J. Am. Chem. Soc. 1992, 114(24), 9369-86 (Andrew G. Myers, Dragovich S. Peter, and Kuo Y. Elaine). A solution of this aldehyde (10.0 g, 28.4 mmol) in toluene (60 mL) was treated with 1-triphenylphosphoranylidene-2-propanone (22.0 g, 63.0 mmol), and the reaction mixture was heated at reflux for 16 h. After cooling to room temperature, the solvent was removed in vacuum and the residue was purified by chromatography over silica gel (eluting Hexane/EtOAc 8:2) to give 6.0 g of ketone 51.

Step 2: To a solution of ketone 51 prepared in Step 1 (6.0 g, 15.3 mmol) in THF (20 mL) at −78° C. was added slowly KHMDS (17.0 mmol, 17.0 mL, 1.0 M in THF). The reaction mixture was stirred at −30° C. for 1 h, cooled to −78° C. and then treated with a solution of TBSCl (3.0 g, 17.0 mmol) in THF (20 mL). The mixture was stirred at −78° C. for 2 h and allowed to warm to room temperature over 16 h. After quenching with saturated aqueous NH₄Cl, the mixture was extracted with EtOAc, dried over Na₂SO₄ and concentrated to yield 7.74 g of diene 52.

Step 3: A mixture of diene 52 prepared in Step 2 (7.6 g, 15.0 mmol), p-chlorobenzensulfonamide (1.44 g, 7.5 mmol), cyclopropanecarboxaldehyde (0.75 g, 10.5 mmol) and THF (5 mL) was heated at reflux for 12 h. After cooling to room temperature the solvent was removed to give a mixture of cis and trans products (cis/trans=2:1), which were separated by flash chromatography (eluting Hexane/EtOAc 8:2) to give 1.50 g of the desired cis sulfonamide 53 as a solid.

Step 4: To a solution of sulfonamide 53 prepared in Step 3 (1.5 g, 2.0 mmol) in DCM (15 mL) at 0° C. was added slowly concentrated HCl (0.75 mL). After stirring at 0° C. for 2 h, the mixture was neutralized with saturated aqueous NaHCO₃, the layers were separated, the organic phase was dried over Na₂SO₄ and concentrated. The residue was purified by chromatography over silca gel (eluting Hexane/EtOAc 9:1) to give 1.2 g of ketone 54 as a white solid.

Step 5: To a solution of ketone 54 prepared in Step 4 (0.97 g, 1.5 mmol) in THF (10 mL) was added CeCl₃.7H₂O (0.12 g) followed by NaBH₄ (0.61 g, mmol). The cooling bath was removed and the reaction mixture was stirred at room temperature for 1 h. The mixture was dilute with water, extracted with EtOAc, dried over Na₂SO₄, and concentrated. The residue was purified by chromatography over silica gel (eluting Hexane/EtOAc 7:3) to give 0.69 g of alcohol 55 as a clear oil.

Step 6: A solution of alcohol 55 prepared in Step 5 (0.691 g, 1.1 mmol), acetic anhydride (10.8 g, 0.106 mmol) and p-touenesulfonic acid monohydrate (60 mg, 0.32 mmol) was stirred at room temperature for 16 h. The reaction mixture was diluted with water and extracted with ethyl acetate. The organic phase was washed with brine, dried over MgSO₄ and concentrated to give 0.67 g of compound 56 as a clear oil.

Step 7: A solution of compound 56 prepared in Step 6 (610 g, 0.9 mmol) in THF (40 mL) was treated with TBAF (1.3 ml, 1.3 mmol, 1M in THF). The reaction mixture was stirred at room temperature for 1 h. After removing the solvent in vacuum, the crude mixture was extracted with EtOAc. The organic phase was washed with water, followed with saturated aqueous NaHCO₃ and dried over Na₂SO₄. The solvent was removed in vacuum and the crude product was purified by flash chromatography (eluting Hexane/EtOAc 7/3) to give 0.386 g of alcohol 57 as a clear oil.

Step 8: To a rapidly stirred solution of alcohol 57 prepared in Step 7 (386 mg, 0.87 mmol) in CH2Cl2 (2 mL) and H₂O (0.5 mL) at 0° C., were subsequently added 4-acetamido-TEMPO (1.8 mg, 0.01 mmol), [CH₃(CH₂)₃]₄N⁺HSO₄⁻ (77 mg, 0.23 mmol) and NaBr (9 mg, 0.09 mmol). Then, aq. NaOCl (0.83 M, 2.1 mL, 1.74 mmol), containing NaHCO₃ (250 mg) was added and the mixture was stirred vigorously for 20 min. The organic solvent was evaporated under reduced pressure, and the residue was taken up with EtOAc (20 mL) and aqueous citric acid (10%, 10 mL) containing KI (60 mg). The aqueous phase was re-extracted with EtOAc and the combined organic phases were washed with aqueous Na₂S₂O₃ and brine and dried (MgSO₄). The organic phase was evaporated under reduced pressure to give 396 mg of acid 58 as a yellow solid.

Step 9: To a solution of acid 58 prepared in Step 8 (395 mg, 0.87 mmol) in MeOH (15 mL) was added K₂CO₃ (723 mg, 5.23 mmol). The mixture was stirred at room temperature for 1 h and the solvent was removed at reduced pressure. The residue was taken up in water, acidified with 1N HCl and extracted with EtOAc. The organic phase was dried (MgSO₄) and concentrated under reduced pressure to give 293 mg of acid 59.

Step 10: To a mixture of acid 50 prepared in Step 9 (50 mg, 0.12 mmol) in 2.0 mL of DMF was added iPr₂NEt (62 mg, 0.48 mmol) and HATU (60 mg, 0.16 mmol). After stirring for 5 min, 2-methyl-2-piperazin-1-yl-propan-1-ol (as the dihydrochloride salt, 43 mg, 0.18 mmol) was added and the mixture was stirred at room temperature for 16 h. The mixture was diluted with EtOAc, washed with water, brine and dried (Na₂SO₄). The organic phase was then loaded on a preparative TLC plate (silica gel) using 5% MeOH in DCM as a solvent to provide 33 mg of Example 45.

Example 45: ¹H NMR (CDCl₃, 300 MHz) δ 7.70 (2H, d, J=8.4 Hz), 7.45 (2H, d, J=8.0 Hz), 4.20 (1H, t, J=8.0 Hz), 3.75-3.40 (4H, m), 3.38-3.28 (3H, m), 3.10-3.0 (1H, m), 2.88-2.78 (1H, m), 2.72-2.45 (4H, m), 1.97-1.75 (4H, m), 1.47-1.35 (1H, m), 1.25-1.10 (3H, m), 1.03 (6H, s), 0.90-0.47 (7H, m), 0.33-0.22 (1H, m). LCMS (ES) Retention time 2.60 min, m/z 554.1 (M+H)⁺.

### PREPARATION OF EXAMPLES 46-49

The following Examples 46-49 were prepared by reacting acid 59 with the appropriate cyclic amine (i.e., rather than with 2-methyl-2-piperazin-1-yl-propan-1-ol) under conditions similar to those described in Step 10, above. Thus, for example, Example 46 was prepared by reacting acid 49 with 4-piperidinopiperidine rather than 2-methyl-2-piperazin-1-yl-propan-1-ol.

| | | Retention time | Observed mass |
|---|---|---|---|
| Example | Structure | (min) | (m/z, M + H) |
| 46 | 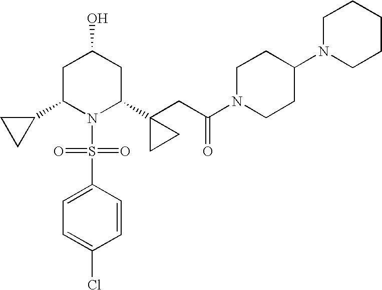 | 2.87 | 564.1 |
| 47 | 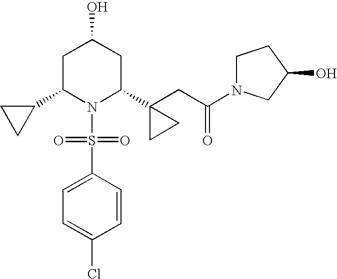 | 2.90 | 483.1 |
| 48 | 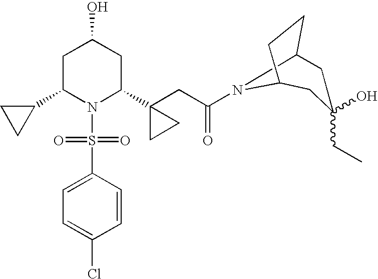 | 3.88 | 551.1 |
| 49 | 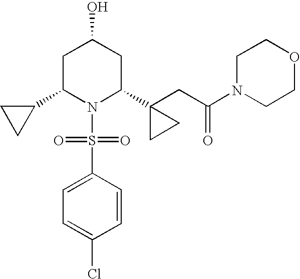 | 3.20 | 483.1 |

Assay

Gamma secretase activity was determined as described by Zhang et al. (Biochemistry, 40 (16), 5049-5055, 2001), herein incorporated by reference in, its entirety. Activity is expressed either as a percent inhibition or as the concentration of compound producing 50% inhibition of enzyme activity.

Reagents

Antibodies W02, G2-10, and G2-11 were obtained from Dr. Konrad Beyreuther (University of Heidelberg, Heidelberg, Germany). W02 recognizes residues 5-8 of Aβ peptide, while G2-10 and G2-11 recognize the specific C-terminal structure of Aβ 40 and Aβ 42, respectively. Biotin-4G8 was purchased from Senetec (St. Louis, Mo.). All tissue culture reagents used in this work were from Life Technologies, Inc., unless otherwise specified. Pepstatin A was purchased from Roche Molecular Biochemicals; DFK167 was from Enzyme Systems Products (Livermore, Calif.).

cDNA Constructs, Tissue Culture, and Cell Line Construction

The construct SPC99-Lon, which contains the first 18 residues and the C-terminal 99 amino acids of APP carrying the London mutation, has been described (Zhang, L., Song, L., and Parker, E. (1999) J. Biol. Chem. 274, 8966-8972). Upon insertion into the membrane, the 17 amino acid signal peptide is processed, leaving an additional leucine at the N-terminus of Aβ. SPC99-lon was cloned into the pcDNA4/TO vector (Invitrogen) and transfected into 293 cells stably transfected with pcDNA6/TR, which is provided in the T-REX system (Invitrogen). The transfected cells were selected in Dulbecco's modified Eagle's media (DMEM) supplemented with 10% fetal bovine serum, 100 units/mL penicillin, 100 g/mL streptomycin, 250 g/mL zeocin, and 5 g/mL blasticidin (Invitrogen). Colonies were screened for Aβ production by inducing C99 expression with 0.1 g/mL tetracycline for 16-20 h and analyzing conditioned media with a sandwich immunoassay (see below). One of the clones, designated as pTRE.15, was used in these studies.

Membrane Preparation

C99 expression in cells was induced with 0.1 g/mL tetracycline for 20 h. The cells were pretreated with 1 M phorbol 12-myristate 13-acetate (PMA) and 1 M brefeldin A (BFA) for 5-6 h at 37° C. before harvesting. The cells were washed 3 times with cold phosphate-buffered saline (PBS) and harvested in buffer A containing 20 mM Hepes (pH 7.5), 250 mM sucrose, 50 mM KCl, 2 mM EDTA, 2 mM EGTA, and Complete protease inhibitor tablets (Roche Molecular Biochemicals). The cell pellets were flash-frozen in liquid nitrogen and stored at −70° C. before use.

To make membranes, the cells were resuspended in buffer A and lysed in a nitrogen bomb at 600 psi. The cell lysate was centrifuged at 1500 g for 10 min to remove nuclei and large cell debris. The supernatant was centrifuged at 100000 g for 1 h. The membrane pellet was resuspended in buffer A plus 0.5 M NaCl, and the membranes were collected by centrifugation at 200000 g for 1 h. The salt-washed membrane pellet was washed again in buffer A and centrifuged at 100000 g for 1 h. The final membrane pellet was resuspended in a small volume of buffer A using a Teflon-glass homogenizer. The protein concentration was determined, and membrane aliquots were flash-frozen in liquid nitrogen and stored at −70° C.

γ-Secretase Reaction and Aβ Analysis To measure γ-secretase activity, membranes were incubated at 37° C. for 1 h in 50 L of buffer containing 20 mM Hepes (pH 7.0) and 2 mM EDTA. At the end of the incubation, Aβ 40 and Aβ 42 were measured using an electrochemiluminescence (ECL)-based immunoassay. Aβ 40 was identified with antibody pairs TAG-G2-10 and biotin-W02, while Aβ 42 was identified with TAG-G2-11 and biotin-4G8. The ECL signal was measured using an ECL-M8 instrument (IGEN International, Inc.) according to the manufacturer's instructions. The data presented were the means of the duplicate or triplicate measurements in each experiment. The characteristics of γ-secretase activity described were confirmed using more than five independent membrane preparations.

Using the above assay, the compounds of Examples 1-49 showed IC₅₀ values within the range of about 0.001 to about 0.5 μM. The compounds of Examples 1-11, 17, and 19-48 showed IC₅₀ values within the range of about 0.001 to about 0.2 μM. The compounds of Examples 1-5, 19-25, 28-30, 32, 33, 36-40, 42, 45, 46, and 48 showed IC₅₀ values within the range of about 0.001 to about 0.02 μM.

The γ-secretase inhibitory activity of some of the inventive compounds are shown below:

| Example | IC₅₀ (μM) |
|---|---|
| 1 | 0.0028 |
| 2 | 0.0164 |
| 3 | 0.0132 |
| 4 | 0.0014 |
| 5 | 0.0196 |
| 19 | 0.0119 |
| 20 | 0.0151 |
| 21 | 0.0117 |
| 22 | 0.0164 |
| 23 | 0.0124 |
| 24 | 0.0145 |
| 25 | 0.0049 |
| 29 | 0.0068 |
| 30 | 0.0025 |
| 32 | 0.0023 |
| 33 | 0.0045 |
| 36 | 0.0067 |
| 38 | 0.0031 |
| 40 | 0.0135 |
| 42 | 0.0085 |
| 45 | 0.0081 |
| 48 | 0.0048 |

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of formula I: 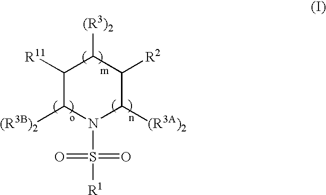
or a pharmaceutically acceptable salt, solvate and/or ester thereof, wherein:
R¹ is selected from the group consisting of unsubstituted aryl, aryl substituted with one or more R⁵ groups, unsubstituted heteroaryl, and heteroaryl substituted with one or more R⁵ groups;
R² is selected from the group consisting of —C(O)—Y, -alkylene-C(O)—Y, -alkylene-cycloalkylene-C(O)—Y, -cycloalkylene-alkylene-C(O)—Y, -alkylene-cycloalkylene-alkylene-C(O)—Y, -cycloalkylene-C(O)—Y, —S(O)—Y, -alkylene-S(O)—Y, -alkylene-cycloalkylene-S(O)—Y, -cycloalkylene-alkylene-S(O)—Y, -alkylene-cycloalkylene-alkylene-S(O)—Y, -cycloalkylene-S(O)—Y, —S(O₂)—Y, -alkylene-S(O₂)—Y, -alkylene-cycloalkylene-S(O₂)—Y, -cycloalkylene-alkylene-S(O₂)—Y, -alkylene-cycloalkylene-alkylene-S(O₂)—Y, and -cycloalkylene-S(O₂)—Y; wherein each of said alkylene or cycloalkylene are unsubstituted or optionally substituted with one or more hydroxy groups, with the proviso that no hydroxy group is bonded to a carbon atom which is also bonded to a sulfur atom;
each R³ of (R³)₂ is independently selected from the group consisting of H, alkyl, —O-alkyl, —OH, —N(R⁹)₂, acyl, and aroyl; or
the moiety (R³)₂, together with the ring carbon atom to which it is shown attached in formula I, defines a carbonyl group, —C(O)—, with the proviso that when m is an integer greater than 1, at most one carbonyl group is present in the ring shown in formula I;
each R^{3A} and R^{3B} is independently selected from the group consisting of H and alkyl;
R⁵ is independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, and —N(R⁶)C(O)NR⁶R⁷;
Y is selected from the group consisting of —NR⁶R⁷, —N(R¹²)(CH₂)_{b}NR⁶R⁷ (wherein b is an integer of from 2-6), aryl, heteroaryl, alkyl, cycloalkyl, heterocycloalkyl, arylalkyl, arylcycloalkyl, heteroarylalkyl, heteroarylcycloalkyl, arylheterocycloalkyl, aryl alkyl heterocycloalkyl, substituted aryl, substituted heteroaryl, substituted arylalkyl, substituted arylcycloalkyl, substituted heteroarylalkyl, substituted heteroarylcycloalkyl, substituted arylheterocycloalkyl, and substituted heterocycloalkyl alkyl; wherein the aryl or heteroaryl moiety in said substituted aryl, substituted heteroaryl, substituted arylalkyl, substituted arylcycloalkyl, substituted heteroarylalkyl, substituted heteroarylcycloalkyl, substituted arylheterocycloalkyl, or substituted heterocycloalkyl alkyl groups of said Y group are substituted with one or more substituents independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, —N(R⁶)C(O)NR⁶R⁷, and alkyl; or
Y is selected from the group consisting of: 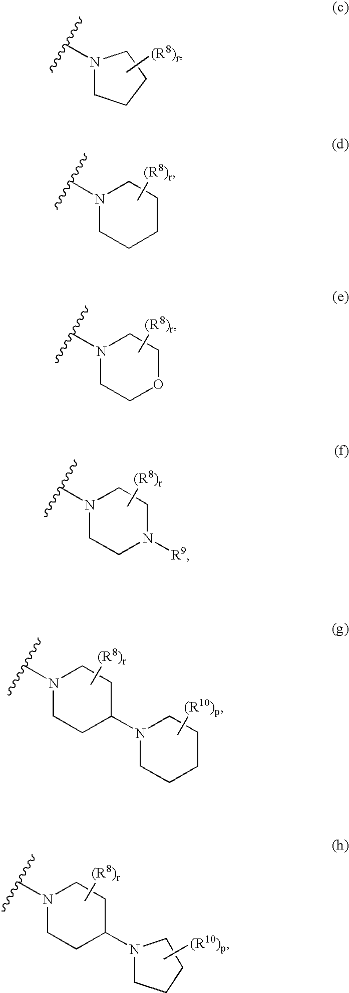 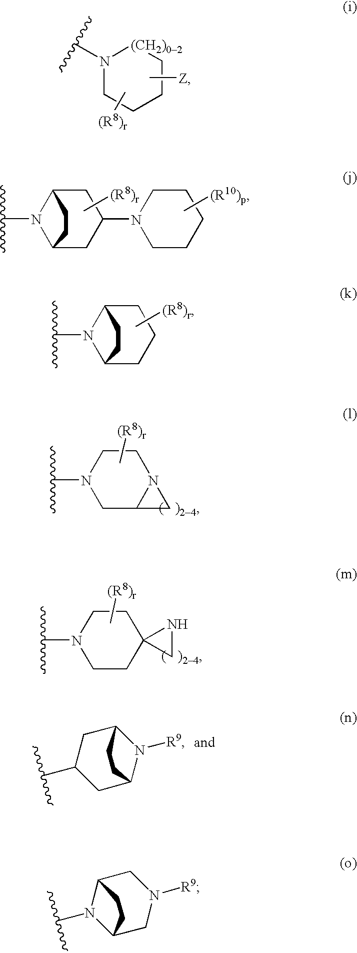
R⁶ and R⁷ are independently selected from the group consisting of H, alkyl, alkyl substituted with 1 to 4 hydroxy groups, cycloalkyl, arylalkyl, heteroarylalkyl, 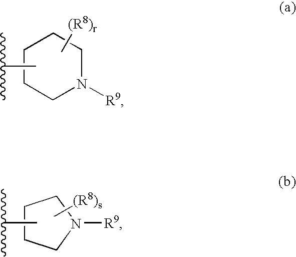
and heterocycloalkyl, with the proviso that if R⁶ and/or R⁷ are alkyl substituted with 1 to 4 hydroxy groups, none of the hydroxy groups are bonded to a carbon to which a nitrogen is also bonded;
R⁸ is independently selected from the group consisting of H, —OH, alkyl, —O-alkyl, alkyl substituted with 1 to 4 hydroxy groups, and —C(O)O-alkyl; or if r is greater than 1 and at least two R⁸ groups are selected from the group consisting of alkyl, —O-alkyl, alkyl substituted with 1 to 4 hydroxy groups, and —C(O)O-alkyl, then the two R⁸ groups, together with the ring carbon atom or atoms to which they are attached, define a ring;
each R⁹ is independently selected from the group consisting of H, alkyl, alkyl substituted with 1 to 4 hydroxy groups, cycloalkyl, cycloalkyl substituted with 1 to 4 hydroxy groups, arylalkyl, heteroarylalkyl, —C(O)O-alkyl, -alkylene-O-alkylene-OH, aryl substituted with one or more R⁵ groups, heteroaryl substituted with one or more R⁵ groups, unsubstituted heteroaryl, unsubstituted aryl, -alkylene-C(O)O-alkyl, —(SO₂)-alkyl, —(SO₂)-aryl, and hydroxyalkyl-O-alkyl, with the proviso that when R⁹ is alkyl substituted with 1 to 4 hydroxy groups, none of the hydroxy groups are bonded to a carbon to which a nitrogen is also bonded;
each R¹⁰ is independently selected from the group consisting of H and alkyl;
R¹¹ is selected from the group consisting of aryl, substituted aryl, heteroaryl, alkyl, cycloalkyl, arylalkyl, arylcycloalkyl, heteroarylalkyl, heteroarylcycloalkyl, arylheterocycloalkyl, alkoxyalkyl, substituted heteroaryl, substituted arylalkyl, substituted arylcycloalkyl, substituted heteroarylalkyl, and substituted arylheterocycloalkyl; wherein the aryl or heteroaryl moiety in said substituted heteroaryl, substituted arylalkyl, substituted arylcycloalkyl, substituted heteroarylalkyl, and substituted arylheterocycloalkyl of said R¹¹ group is substituted with one or more substituents independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, and —N(R⁶)C(O)NR⁶R⁷;
R¹² is selected from the group consisting of H, alkyl, aryl, and aryl substituted with one or more substituents independently selected from the group consisting of halo, —CF₃, —OH, alkoxy, —OCF₃, —CN, —NH₂, —C(O)O-alkyl, —OC(O)-alkyl, —C(O)O-aryl, —OC(O)-aryl, —C(O)NR⁶R⁷, -alkylene-NR⁶R⁷, —N(R⁶)C(O)-alkyl, —N(R⁶)C(O)-aryl, —N(R⁶)C(O)-heteroaryl, and —N(R⁶)C(O)NR⁶R⁷;
m is an integer of from 0 to 3, and if m is greater than 1, the m moieties can be the same or different from one another;
n is an integer of from 0 to 3, and if n is greater than 1, the n moieties can be the same or different from one another;
o is an integer of from 0 to 3, and if o is greater than 1, the o moieties can be the same or different from one another;
with the proviso that m+n+o is 1, 2, 3 or 4;
p is an integer of from 0 to 4, and if p is greater than 1, the p moieties can be the same or different from one another;
r is an integer of from 0 to 4, and if r is greater than 1, the r moieties can be the same or different from one another;
s is an integer of from 0 to 3, and if s is greater than 1, the s moieties can be the same or different from one another; and
Z is selected from the group consisting of heterocycloalkyl, substituted heterocycloalkyl, —NH₂, —NH(alkyl), —N(alkyl)₂ wherein each alkyl is the same or different, —NH(cycloalkyl), —NH(substituted cycloalkyl), —N(alkyl)(cycloalkyl), —N(alkyl)(substituted cycloalkyl), —NH(aralkyl), —NH(substituted aralkyl), —N(alkyl)(aralkyl), —NH(heterocycloalkyl), —NH(substituted heterocycloalkyl), —N(alkyl)(heterocycloalkyl), —N(alkyl)(substituted heterocycloalkyl), —NH(heteroaralkyl), —NH(substituted heteroaralkyl), —NH-alkylene-(cycloalkyl), —NH-alkylene-(substituted cycloalkyl), —N(alkyl)-alkylene-(cycloalkyl), —N(alkyl)-alkylene-(substituted cycloalkyl), —NH-alkylene-(heterocycloalkyl), —NH-alkylene-(substituted heterocycloalkyl), —N(alkyl)-alkylene-(heterocycloalkyl), —N(alkyl)-alkylene-(substituted heterocycloalkyl), benzo-fused heterocycloalkyl, substituted benzo-fused heterocycloalkyl, H, and —N(hydroxyalkyl)₂, wherein each alkyl may be the same or different; wherein said substituted cycloalkyl, substituted heterocycloalkyl, substituted aryl, or substituted heteroaryl moiety of group Z is substituted with one or more substituents independently selected from the group consisting of alkyl, —OH, alkoxy, —OC(O)-alkyl, —OC(O)-aryl, —NH₂, —NH(alkyl), —N(alkyl)₂ wherein each alkyl is the same or different, —NHC(O)-alkyl, —N(alkyl)C(O)-alkyl, —NHC(O)-aryl, —N(alkyl)C(O)-aryl, —C(O)-alkyl, —C(O)-aryl, —C(O)NH₂, —C(O)NH(alkyl), —C(O)N(alkyl)₂ wherein each alkyl is the same or different, —C(O)O-alkyl, -alkylene-C(O)O-alkyl, piperidinyl, pyrrolidinyl, aryl, heteroaryl, and —O—CH₂CH₂—O— wherein both oxygen atoms are bound to the same carbon atom, and provided that the aryl and heteroaryl moieties of said Z group are not substituted with said —O—CH₂CH₂—O— group.

2. The compound according to claim 1, wherein R² is selected from the group consisting of —(C₀-C₁₂)alkylene-C(O)—Y, —(C₀-C₆)alkylene-(C₃-C₆)cycloalkylene-(C₀-C₆)alkylene-C(O)—Y, —(C₀-C₁₂)alkylene-S(O₂)—Y, and —(C₀-C₆)alkylene-(C₃-C₆)cycloalkylene-(C₀-C₆)alkylene-S(O₂)—Y; wherein alkylene or cycloalkylene is optionally substituted with one or more hydroxy groups, with the proviso that no hydroxy group is bonded to a carbon atom which is also bonded to a sulfur atom;
Y is selected from the group consisting of: 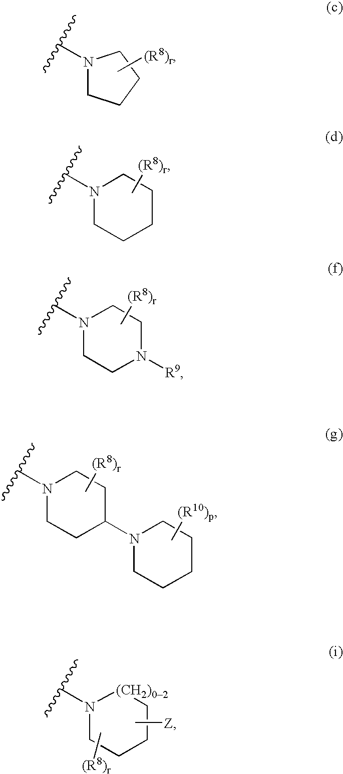
and —NR⁶R⁷;
each R³ of (R³)₂ is independently selected from the group consisting of H, —OH, (C₁-C₆)alkyl, (C₁-C₆)acyl, and (C₇-C₁₃)aroyl; or
the moiety (R³)₂ together with the ring carbon to which it is shown attached in formula I defines a carbonyl group, with the proviso that when m is an integer greater than 1, at most one carbonyl group is present in the ring shown in formula I;
each R^{3A} and R^{3B} is independently selected from the group consisting of H and (C₁-C₆)alkyl;
R⁵ is independently selected from the group consisting of halo, —OH, —CF₃, and —O—(C₁-C₆)alkyl;
R¹¹ is selected from the group consisting of (C₆-C₁₂)aryl, substituted (C₆-C₁₂)aryl, (C₆-C₁₂)heteroaryl, and substituted (C₆-C₁₂)heteroaryl, wherein said substituted (C₆-C₁₂)aryl and substituted (C₆-C₁₂)heteroaryl are substituted with one or more halo, —CF₃, —OH, or —O—(C₁-C₆)alkyl groups;
m is 0 or 1;
n is 0 or 1; and
o is 0 or 1.

3. The compound according to claim 1, wherein R² is selected from the group consisting of —(C₀-C₁₂)alkylene-C(O)—Y, —(C₀-C₆)alkylene-(C₃-C₆)cycloalkylene-(C₀-C₆)alkylene-C(O)—Y, —(C₀-C₁₂)alkylene-S(O)—Y, —(C₀-C₆)alkylene-(C₃-C₆)cycloalkylene-(C₀-C₆)alkylene-S(O)—Y, —(C₀-C₁₂)alkylene-S(O₂)—Y, and —(C₀-C₆)alkylene-(C₃-C₆)cycloalkylene-(C₀-C₆)alkylene-S(O₂)—Y.

4. The compound according to claim 1, wherein R² is —(C₃-C₆)cycloalkylene-C(O)—Y.

5. The compound according to claim 1, wherein R² is -cyclopropylene-C(O)—Y.

6. The compound according to claim 1, wherein R² is —(C₃-C₆)cycloalkylene-(C₀-C₆)alkylene-C(O)—Y.

7. The compound according to claim 1, wherein R² is -cyclopropylene-(C₀-C₆)alkylene-C(O)—Y.

8. The compound according to claim 1, wherein R² is -cyclopropylene-CH₂—C(O)—Y.

9. The compound according to claim 1, wherein R² is -cyclopropylene-CH(OH)—C(O)—Y.

10. The compound according to claim 1, wherein R² is -cyclopropylene-S(O₂)—Y.

11. The compound according to claim 1, wherein R² is —(C₃-C₆)cycloalkylene-(C₀-C₆)alkylene-S(O₂)—Y.

12. The compound according to claim 1, wherein R² is -cyclopropylene-CH₂—S(O₂)—Y.

13. The compound according to claim 3, wherein Y is selected from the group consisting of: 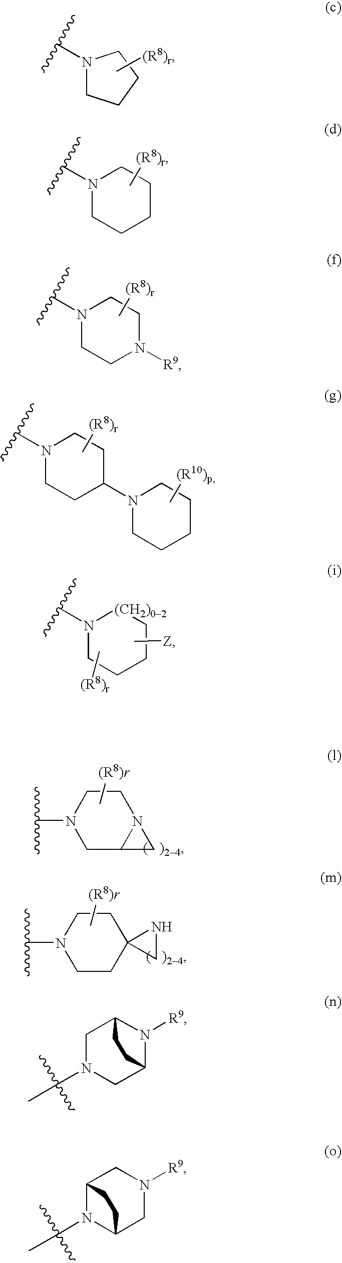
and —NR⁶R⁷.

14. The compound according to claim 1, wherein Y is selected from the group consisting of: 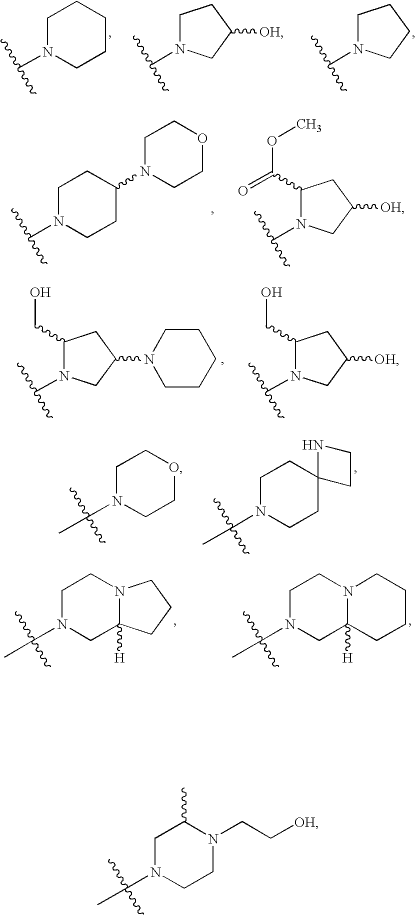
and —N(CH₂CH₂OH)₂.

15. The compound according to claim 1, wherein Y is:

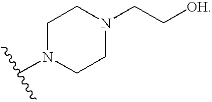

16. The compound according to claim 1, wherein Y is:

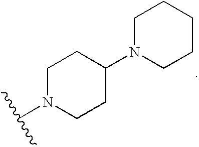

17. The compound according to claim 1, wherein Y is:

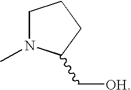

18. The compound according to claim 1, wherein Y is:

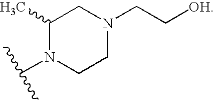

19. The compound according to claim 1, wherein Y is:

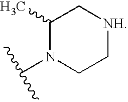

20. The compound according to claim 1, wherein Y is:

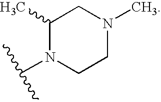

21. The compound according to claim 1, wherein Y is:

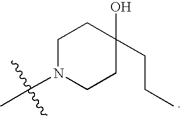

22. The compound according to claim 1, wherein Y is:

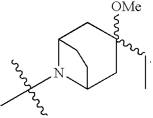

23. The compound according to claim 1, wherein Y is:

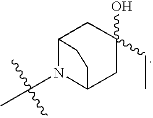

24. The compound according to claim 1, wherein Y is:

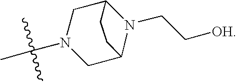

25. The compound according to claim 1, wherein Y is:

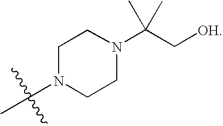

26. The compound according to claim 1, wherein R² is selected from the group consisting of:

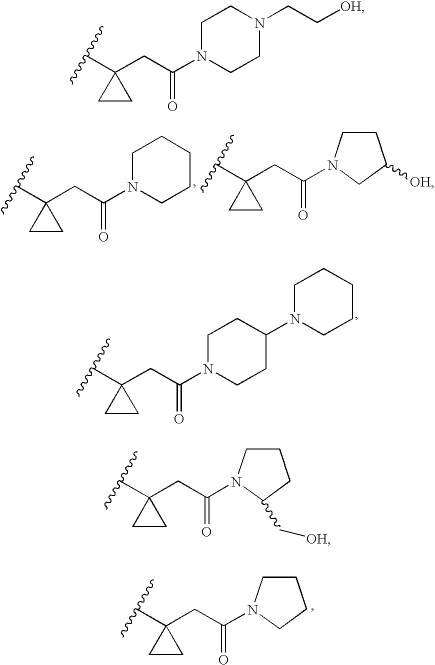

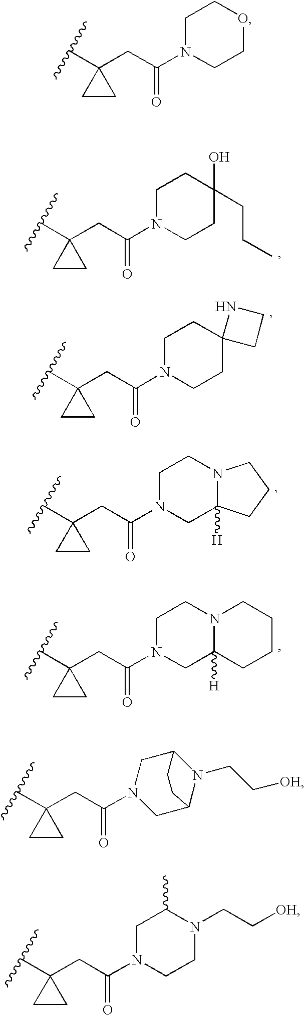

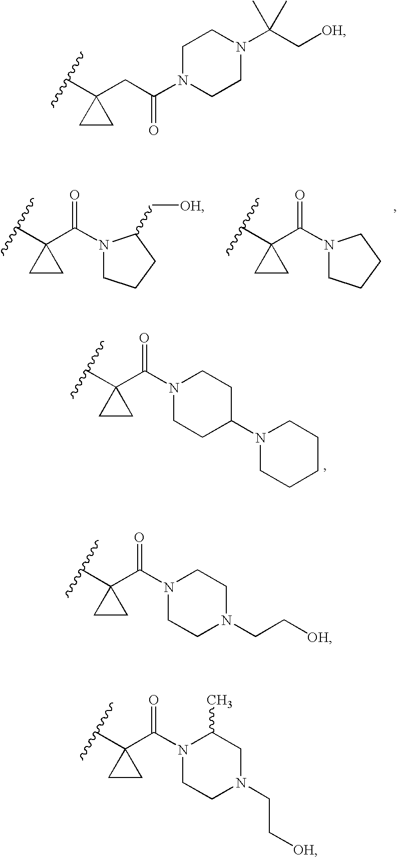

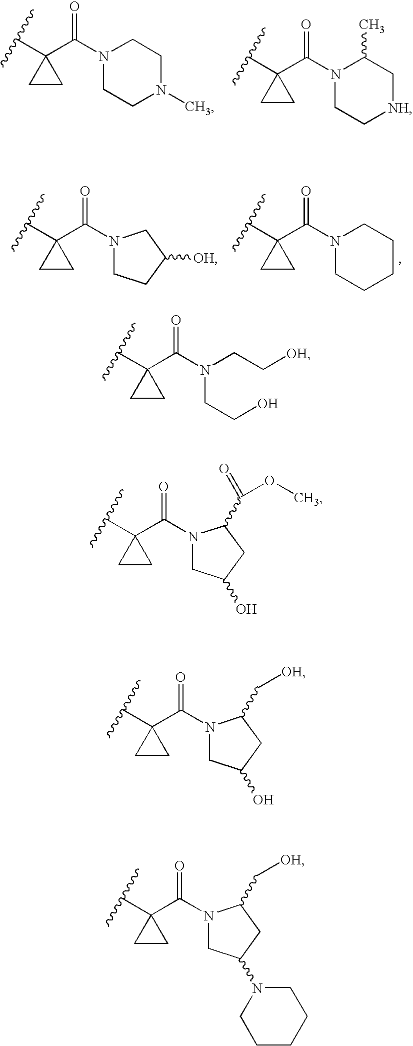

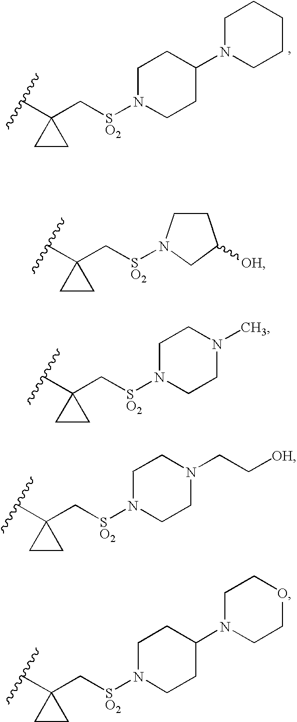

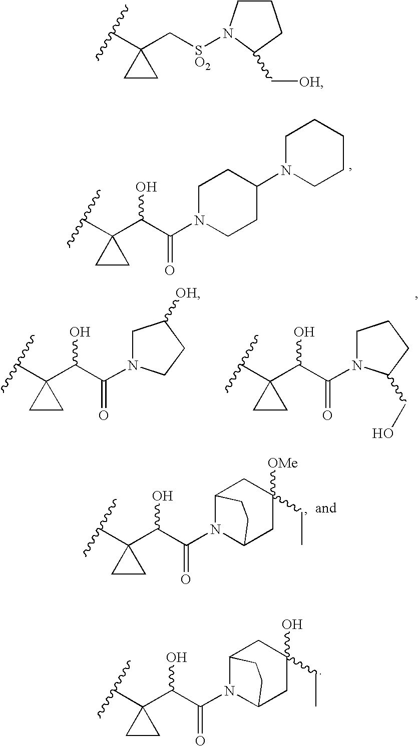

27. The compound according to claim 1, selected from the group consisting of compounds having one of the following structural formulae:

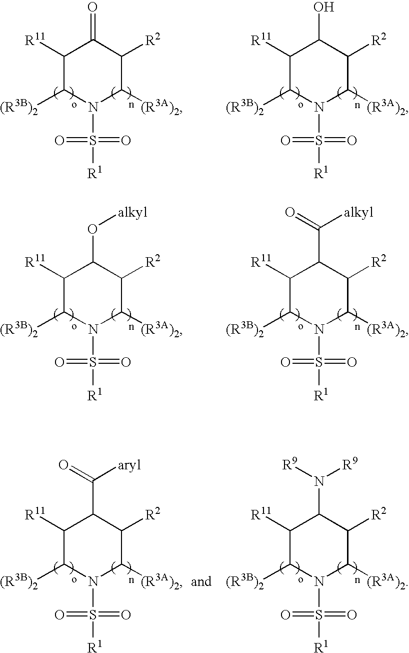

28. The compound according to claim 1, selected from the group consisting of compounds having one of the following structural formulae:

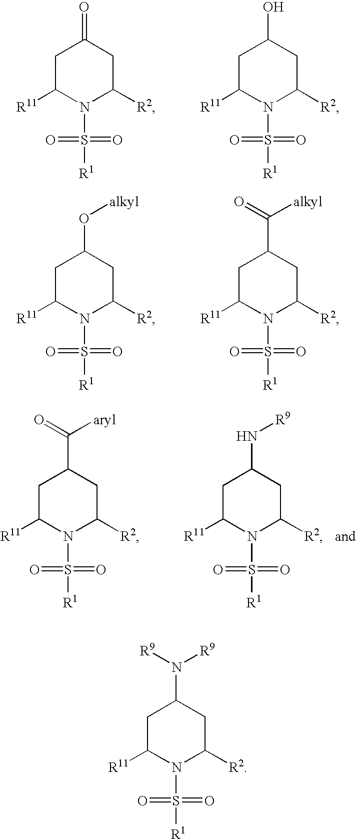

29. The compound according to claim 1, selected from the group consisting of compounds having the following structures: 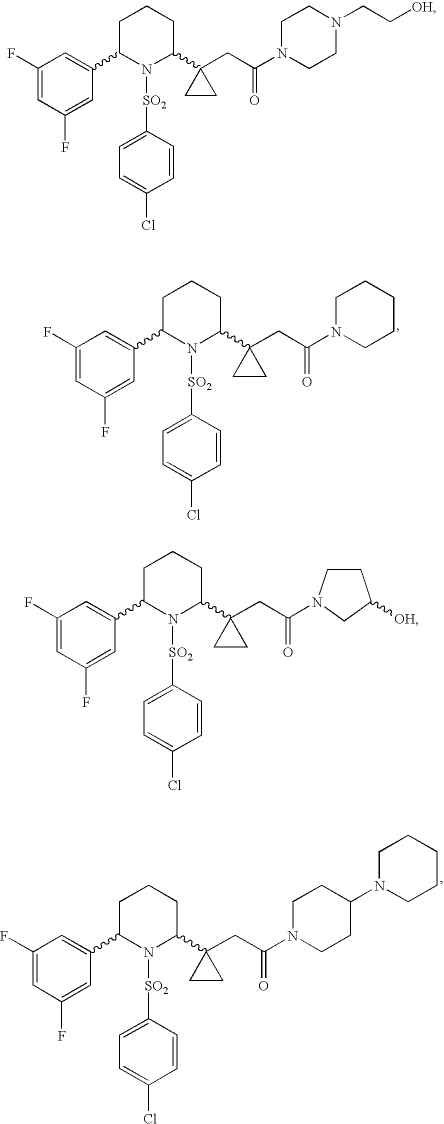 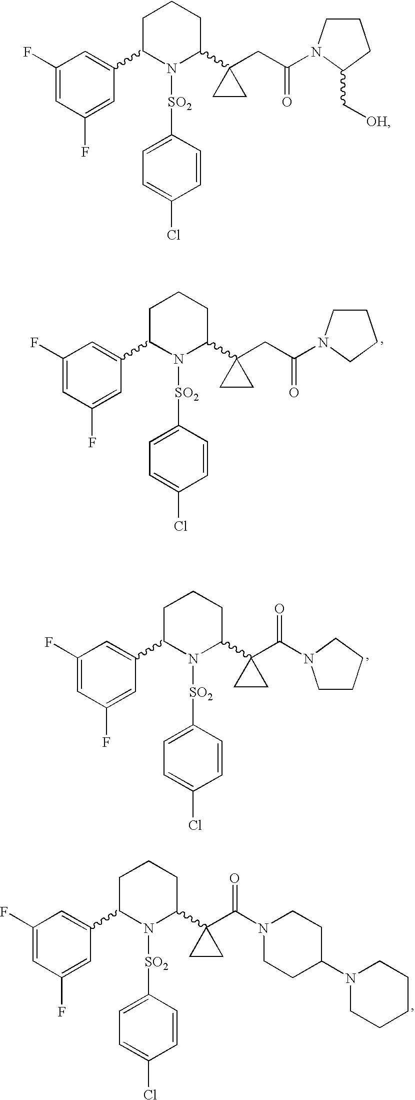 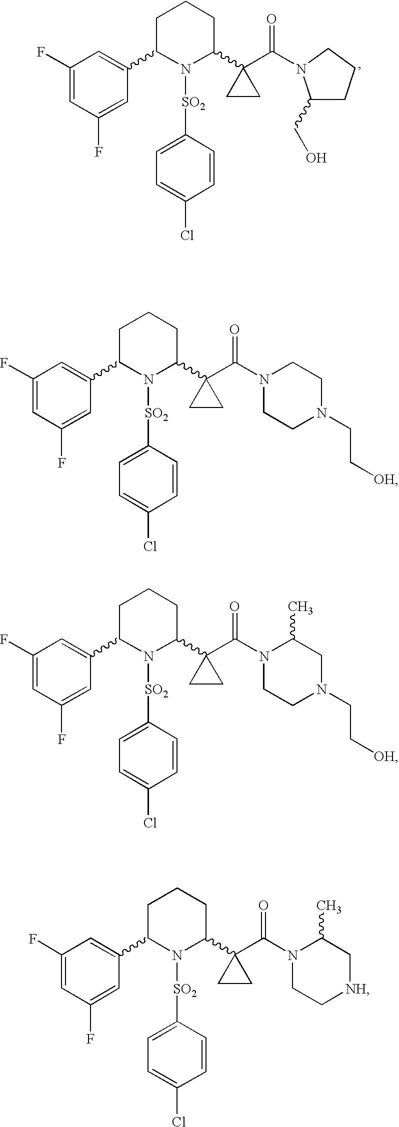 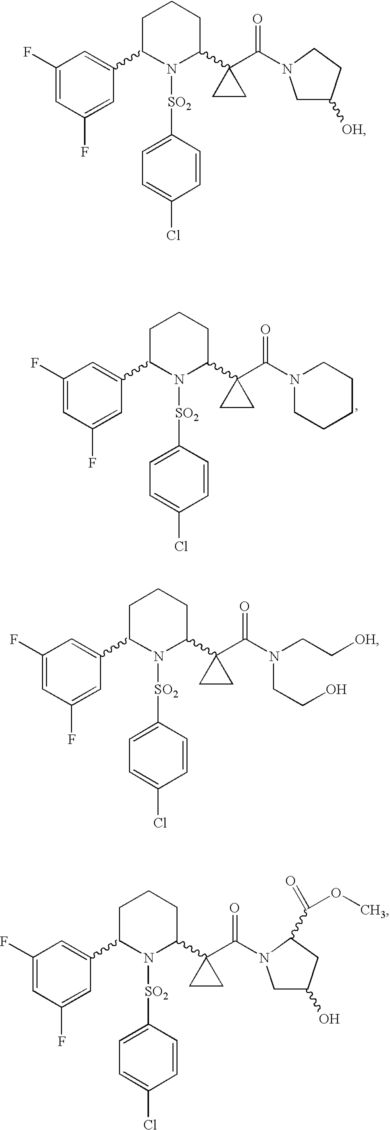 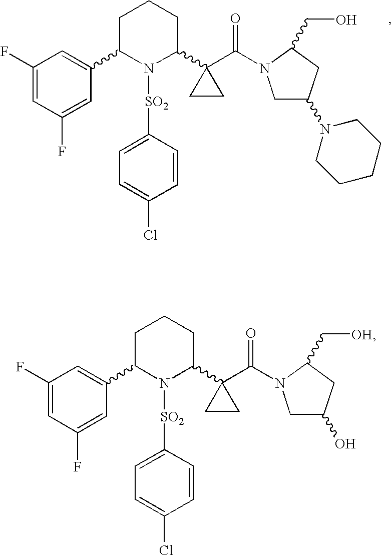 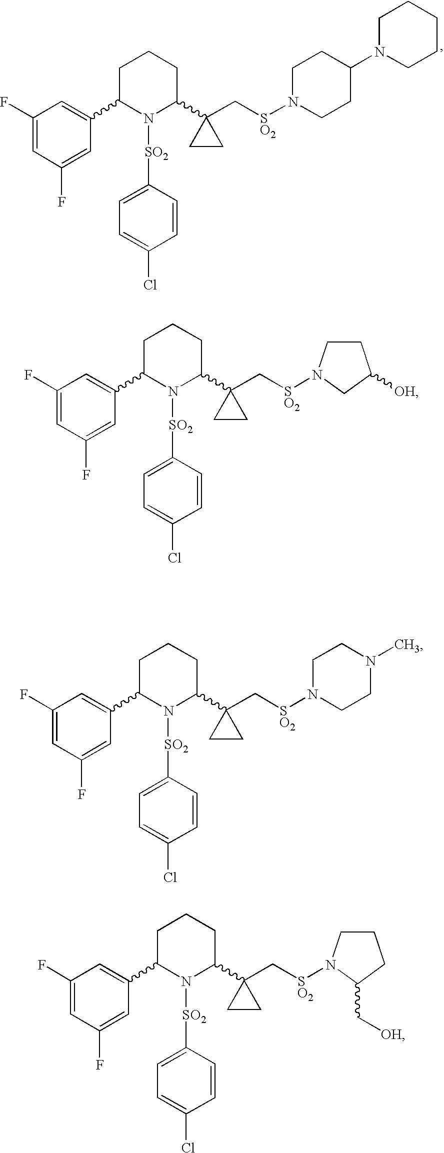 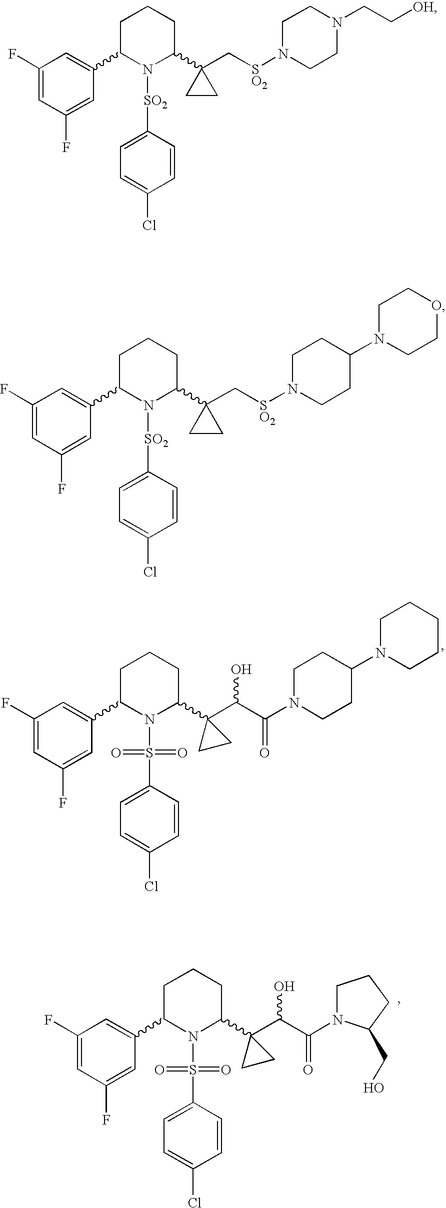 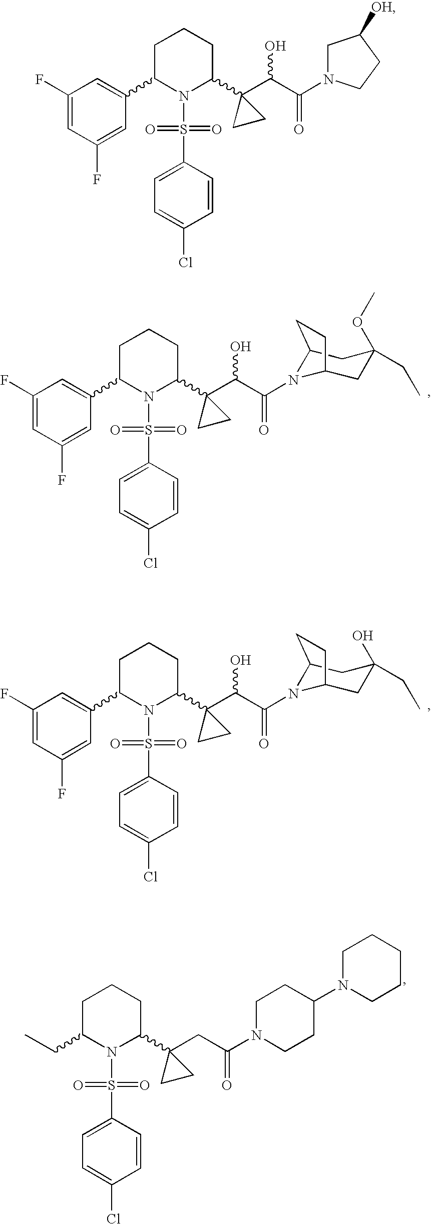 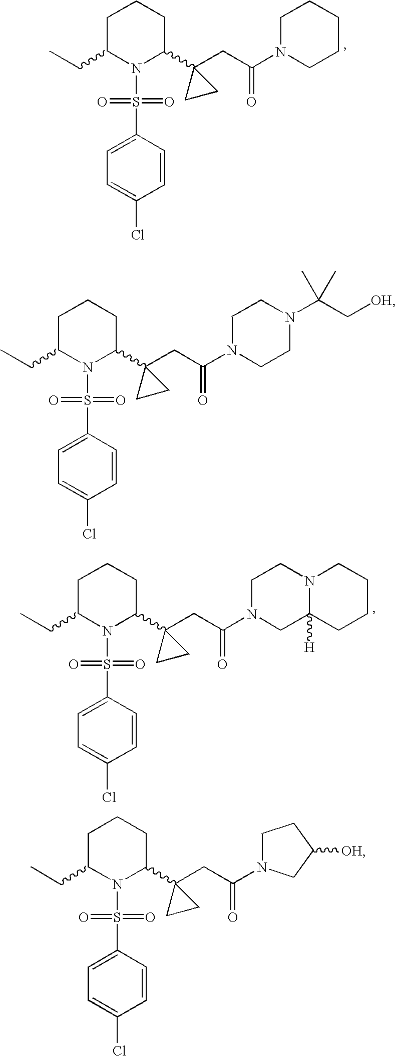 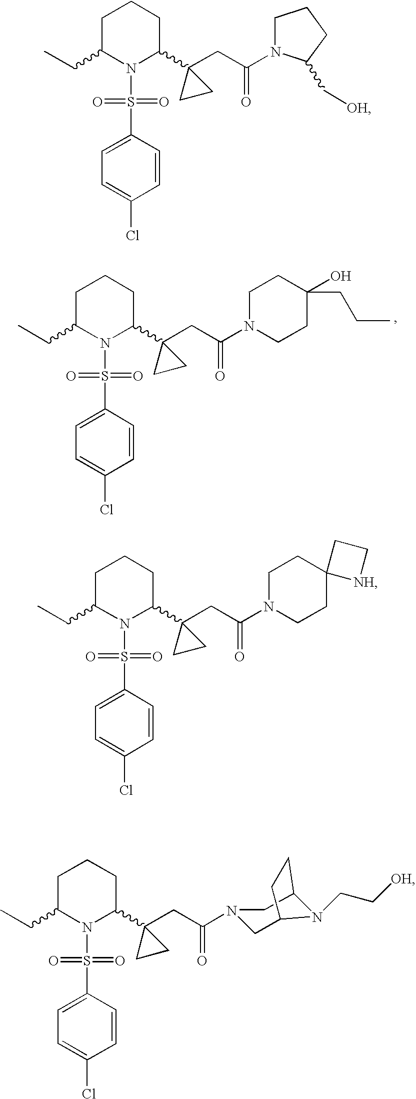 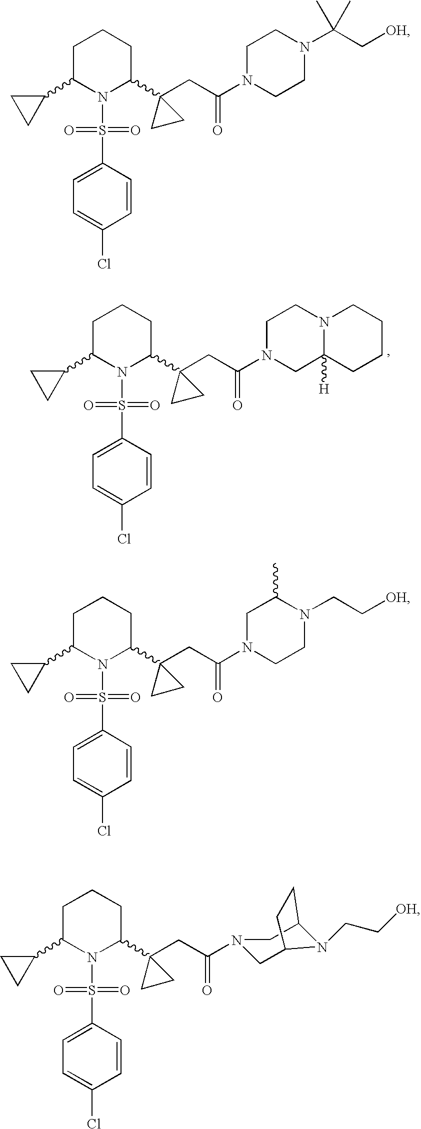 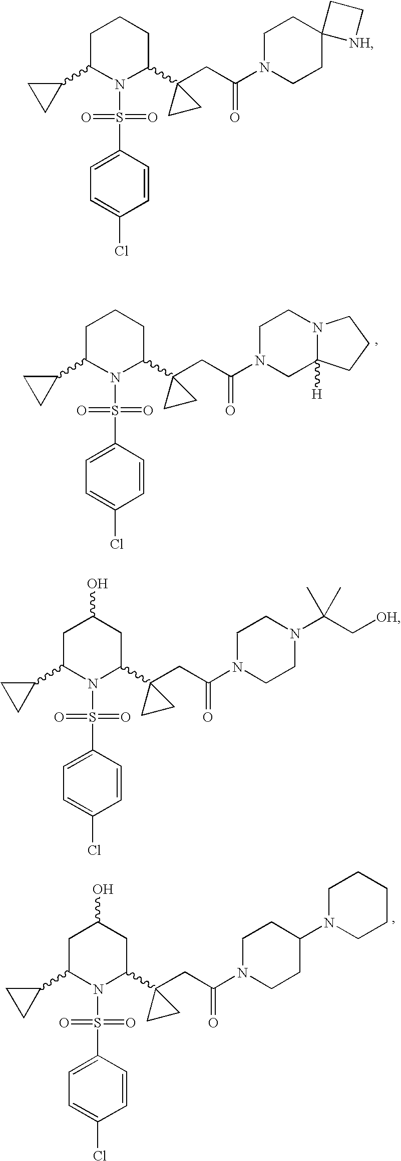 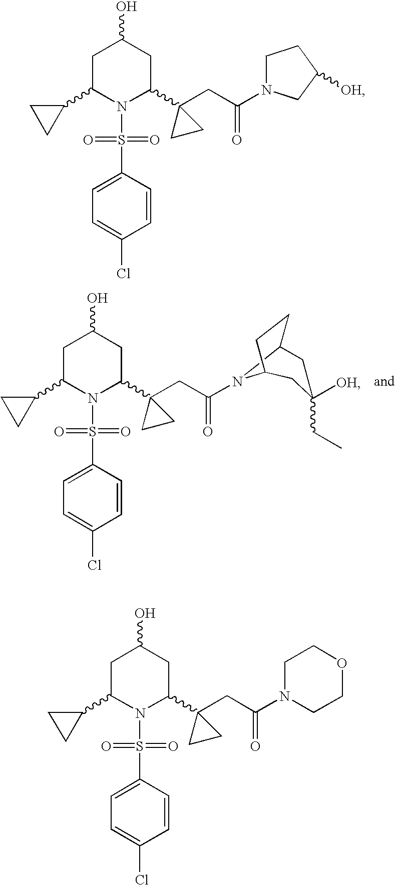
or a pharmaceutically acceptable salt, solvate, and/or ester thereof.

30. The compound according to claim 1, selected from the group consisting of compounds having the following structures: 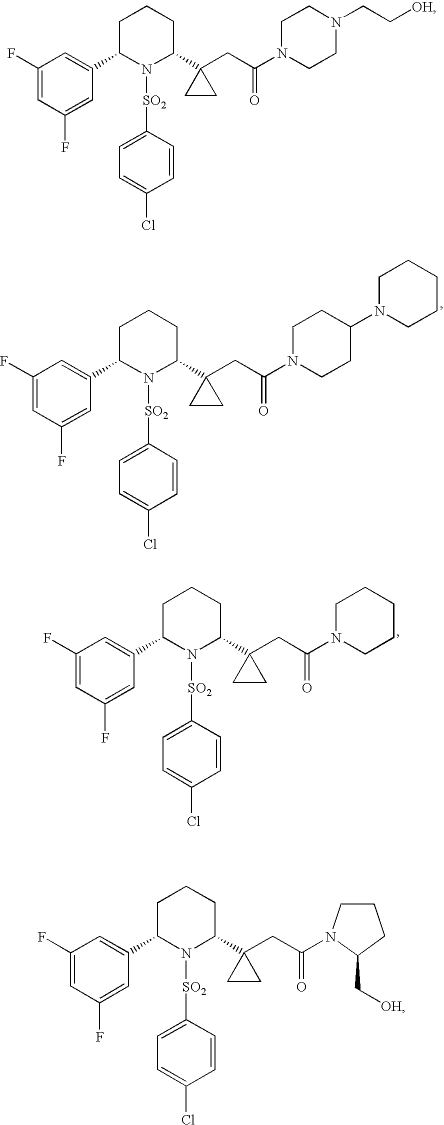 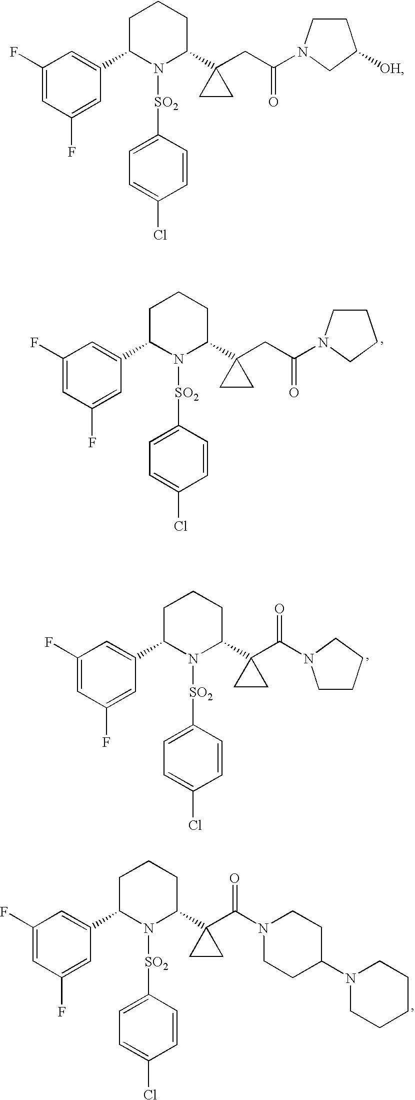 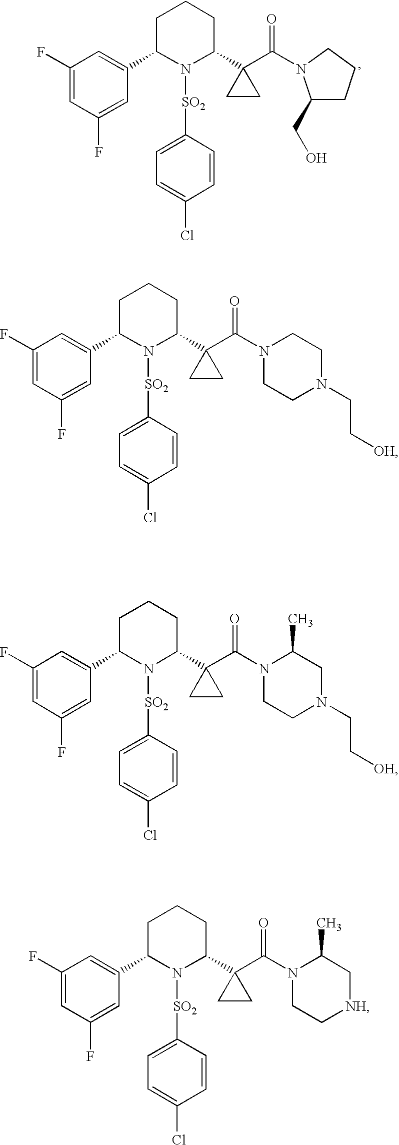 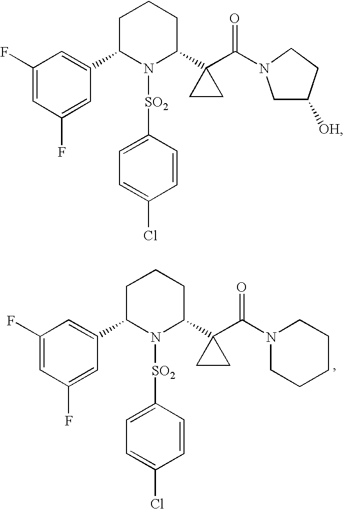 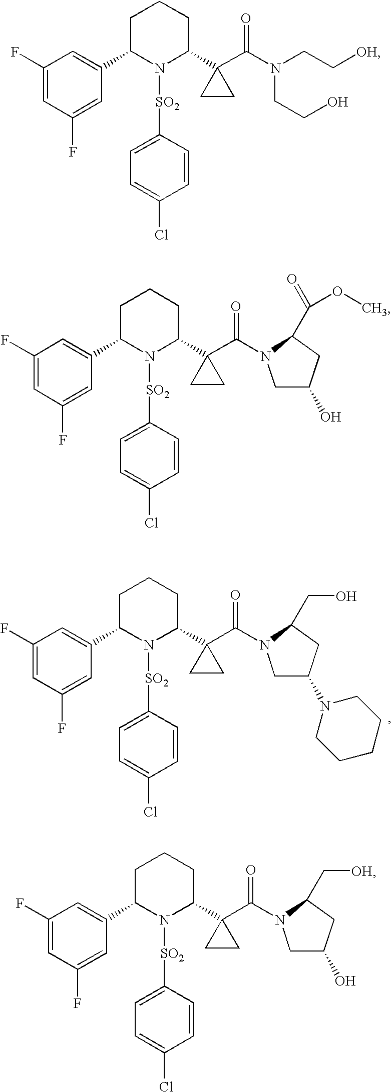 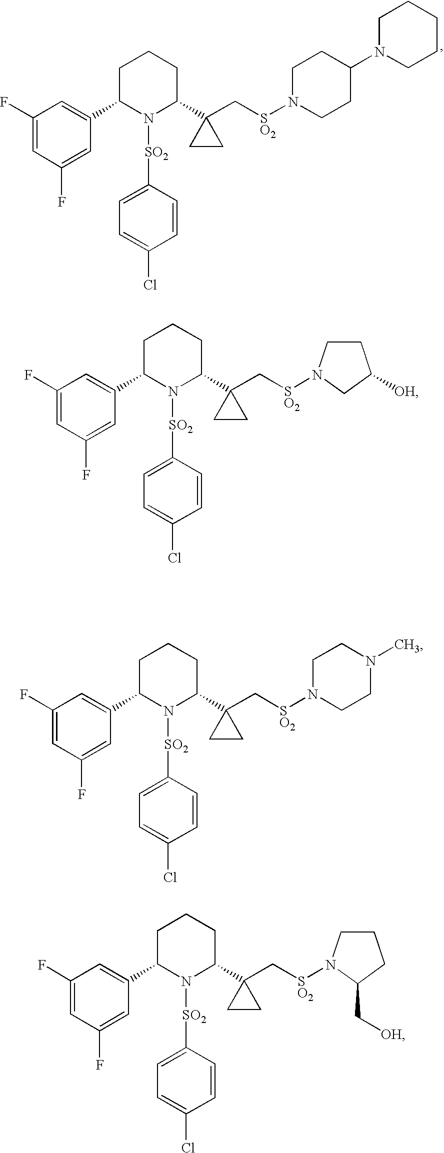 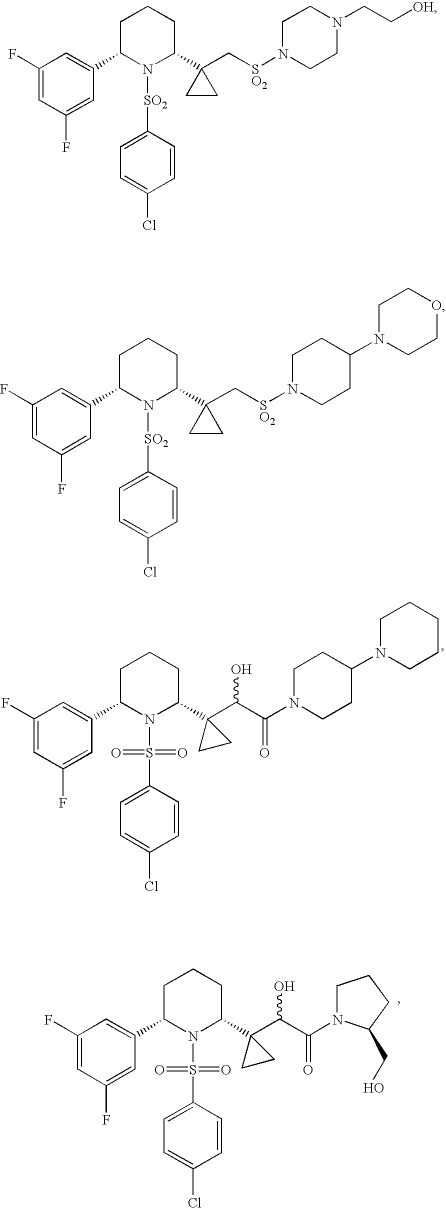 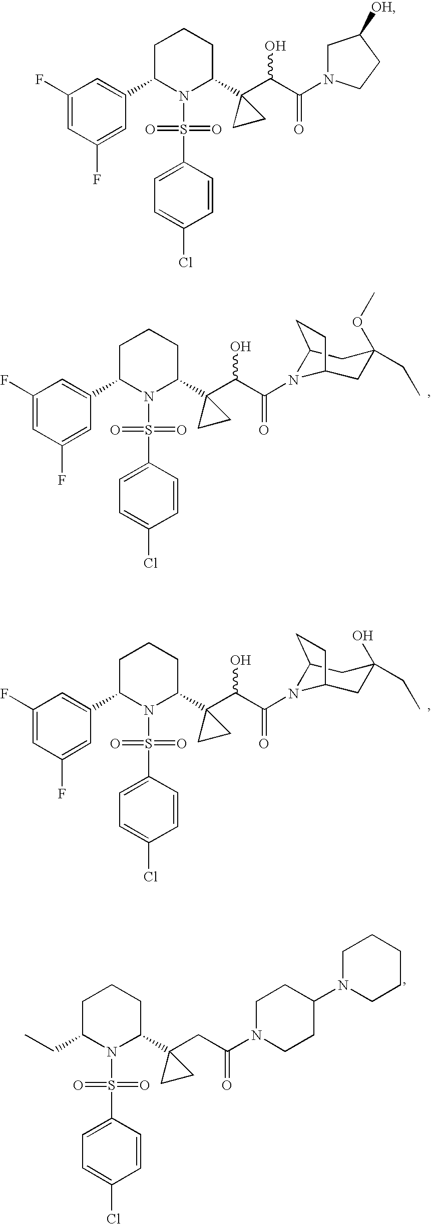 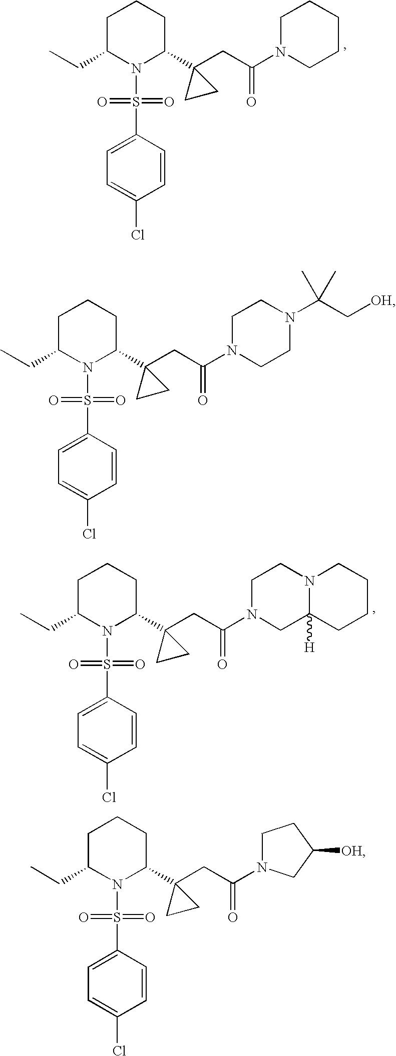 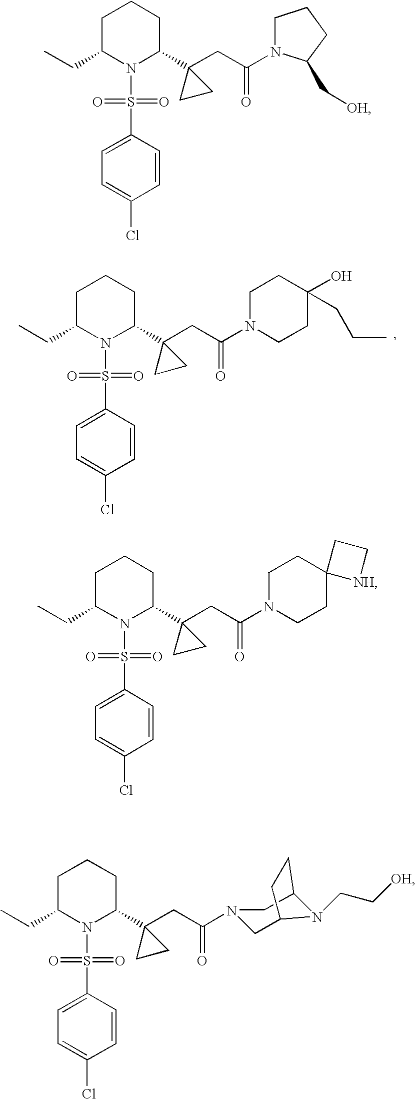 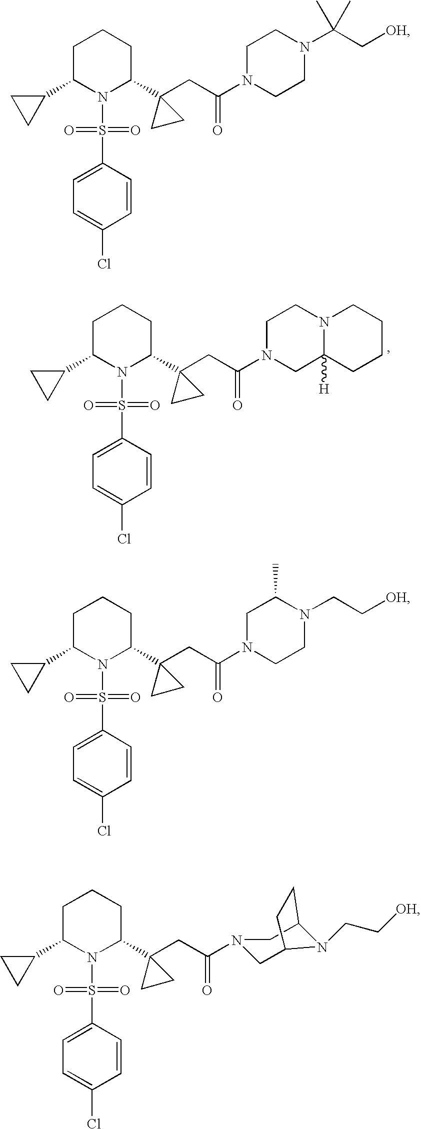 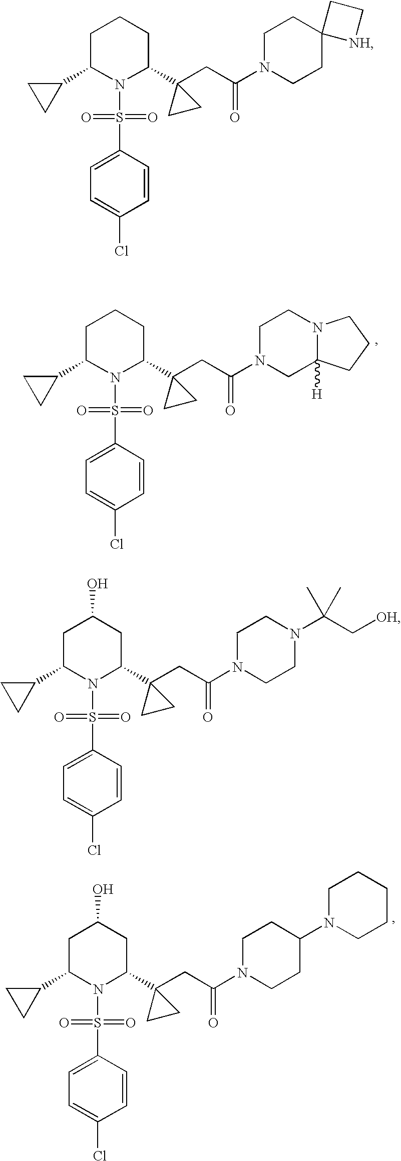 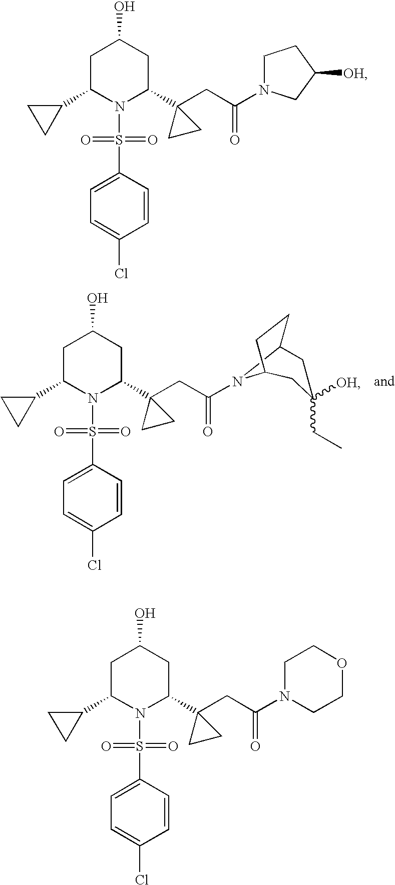
or a pharmaceutically acceptable salt, solvate, and/or ester thereof.

31. A compound of the following structural formula: 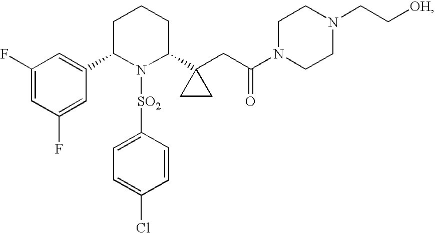
or a pharmaceutically acceptable salt, solvate, and/or ester thereof.

32. A compound of the following structural formula: 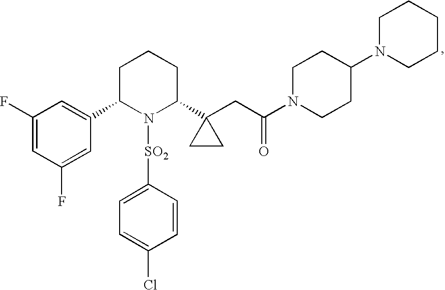
or a pharmaceutically acceptable salt and/or solvate thereof.

33. A compound of the following structural formula: 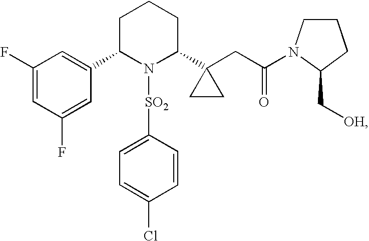
or a pharmaceutically acceptable salt, solvate, and/or ester thereof.

34. A compound of the following structural formula: 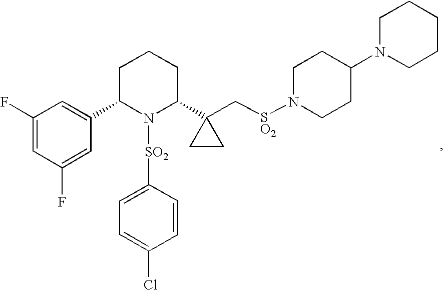
or a pharmaceutically acceptable salt, solvate, and/or ester thereof.

35. A compound of the following structural formula: 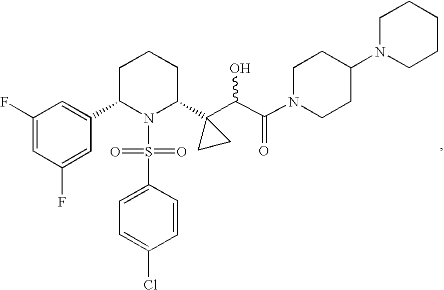
or a pharmaceutically acceptable salt, solvate, and/or ester thereof.

36. A compound of the following structural formula: 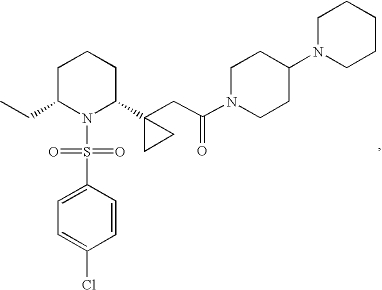
or a pharmaceutically acceptable salt, solvate, and/or ester thereof.

37. A compound of the following structural formula: 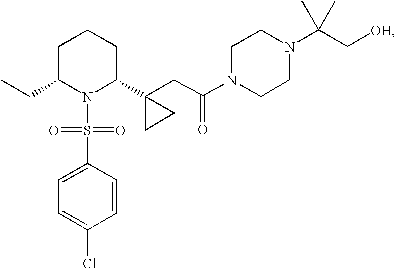
or a pharmaceutically acceptable salt, solvate, and/or ester thereof.

38. A compound of the following structural formula: 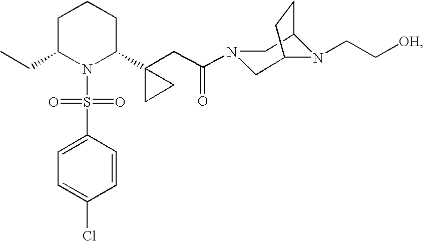
or a pharmaceutically acceptable salt, solvate, and/or ester thereof.

39. A compound of the following structural formula: 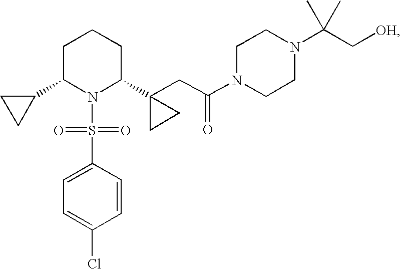
or a pharmaceutically acceptable salt, solvate, and/or ester thereof.

40. A compound of the following structural formula: 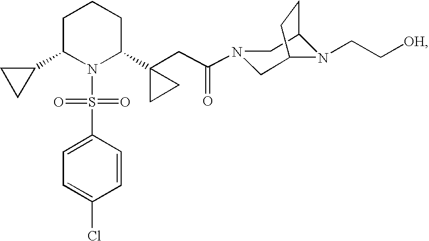
or a pharmaceutically acceptable salt, solvate, and/or ester thereof.

41. A compound of the following structural formula: 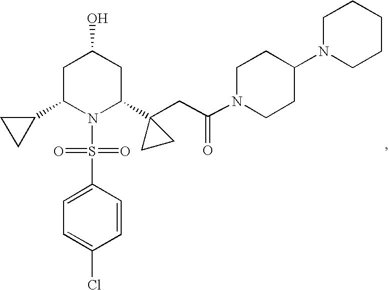
or a pharmaceutically acceptable salt, solvate, and/or ester thereof.

42. A compound of the following structural formula: 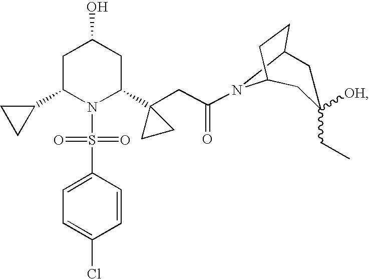
or a pharmaceutically acceptable salt, solvate, and/or ester thereof.

43. A pharmaceutical composition comprising a therapeutically effective amount of a compound of claim 1, or a pharmaceutically acceptable salt, ester and/or solvate thereof, together with at least one pharmaceutically acceptable excipient, diluent or carrier.

44. A pharmaceutical composition comprising a therapeutically effective amount of a compound of claim 30, or a pharmaceutically acceptable salt, ester and/or solvate thereof, together with at least one pharmaceutically acceptable excipient, diluent or carrier.

45. A method of inhibiting gamma-secretase in a patient in need of such treatment comprising administering to said patient a therapeutically effective amount of one or more compounds of claim 1.

46. A method of treating one or more neurodegenerative diseases in a patient in need of such treatment comprising administering to said patient a therapeutically effective amount of one or more compounds of claim 1.

47. A method of inhibiting the deposition of beta amyloid protein in a patient in need of such treatment comprising administering to said patient a therapeutically effective amount of one more compounds of claim 1.

48. A method of treating Alzheimer's disease in a patient in need of such treatment comprising administering to said patient a therapeutically effective amount of one or more compounds of claim 1.

49. A compound of claim 1 in purified form.
